(19) United States
(12) Patent Application Publication
(10) Pub. No.: US 2025/0204942 A1
(43) Pub. Date: Jun. 26, 2025

(54) INTRAVASCULAR LITHOTRIPSY ALGORITHM FOR IMPROVED BALLOON DURABILITY

(71) Applicant: Cardiovascular Systems, Inc., Santa Clara, CA (US)

(72) Inventors: J. Samuel BATCHELDER, Woodinville, WA (US); John R. BALLARD, Waconia, MN (US); Jacob T. WILLIAMS, Plymouth, MN (US); Jason W. STAAB, Apple Valley, MN (US); Donald D. HANSON, Shakopee, MN (US); Thomas D. BRINDLEY, Plymouth, MN (US); Scott P. BOESHART, Plymouth, MN (US); Jeffrey T. MORIARTY, Roseville, MN (US); Austin P. PETRONACK, Plymouth, MN (US); Tim HIDANI, Mound, MN (US); Alex THOME, Minnetonka, MN (US); Daniel SCHMITT, Belle Plaine, MN (US); Kevin CORNELIUS, Wayzata, MN (US); Eric KASEN, Bismarck, ND (US); Sam GARDNER, Crystal, MN (US); Michelle PETERSON, Champlin, MN (US); Khama CHAP, Corcoran, MN (US); Matthew W. TILSTRA, Rogers, MN (US)

(21) Appl. No.: 18/991,526

(22) Filed: Dec. 21, 2024

Related U.S. Application Data
(63) Continuation-in-part of application No. 18/825,683, filed on Sep. 5, 2024.
(63) Continuation-in-part of application No. PCT/US2023/079209, filed on Nov. 9, 2023.
(63) Continuation-in-part of application No. PCT/US2023/085868, filed on Dec. 23, 2023.
(60) Provisional application No. 63/424,573, filed on Nov. 11, 2022.
(60) Provisional application No. 63/580,547, filed on Sep. 5, 2023.
(60) Provisional application No. 63/477,007, filed on Dec. 23, 2022.
(60) Provisional application No. 63/650,872, filed on May 22, 2024.

(51) Int. Cl. A61B 17/22; A61B 17/00; G16H 40/63

(52) U.S. Cl. CPC A61B 17/22022; A61B 17/00234; G16H 40/63; A61B 2017/00172; A61B 2017/00181; A61B 2017/00238; A61B 2017/00292; A61B 2017/00862; A61B 2017/22025; A61B 2017/22062; A61B 2017/22065

(57) ABSTRACT

Various embodiments of the systems, methods, and devices are provided for controlled operation of IVL for breaking up calcified lesions in an anatomical conduit. More specifically, control arrangements are disclosed concerning managing and/or providing electrical energy to generate an electrical arc between a set of spaced-apart electrodes disposed within a fluid-filled balloon, creating a determined pressure output over several voltage pulses.

### CROSS-REFERENCE TO RELATED APPLICATIONS

This application is a Continuation-in-Part of U.S. patent application Ser. No. 18/825,683 filed on Sep. 5, 2024, entitled “Control of IVL Systems, Methods and Devices,” and a Continuation-in-Part of Patent Cooperation Treaty (PCT) Application Serial No. PCT/US23/079209 filed on Nov. 9, 2023, entitled “Control of IVL Systems, Devices and Methods Thereof,” which claims the benefit of and priority to U.S. Provisional Patent Application Ser. No. 63/424,573 filed on Nov. 11, 2022 and entitled “Devices, Systems, and Methods of Intravascular Lithotripsy,” and also claims the benefit of and priority to U.S. Provisional Patent Application Ser. No. 63/580,547 filed on Sep. 5, 2023 and entitled “Devices, Systems, and Methods of Intravascular Lithotripsy.” This application is also a Continuation-in-Part of Patent Cooperation Treaty (PCT) Application Serial No. PCT/US23/085868 filed on Dec. 23, 2023, entitled “Intravascular Lithoplasty System with Improved Durability, Efficiency and Pressure Output Variability,” which claims the benefit of and priority to U.S. Provisional Patent Application Ser. No. 63/477,007 filed Dec. 23, 2022, entitled “Intravascular Lithoplasty and/or Angioplasty Balloon System with Improved Balloon Mount and Electrodes,” and also claims the benefit of and priority to U.S. Provisional Patent Application Ser. No. 63/650,872 filed on May 22, 2024 and entitled “Control of IVL Systems, Methods and Devices,” which applications are expressly incorporated herein by reference in their entirety.

### BACKGROUND OF THE INVENTION

### Field of the Invention

The present disclosure relates to devices, systems, and methods for breaking up calcified lesions in an anatomical conduit. More specifically, the present disclosure relates to devices, systems, and methods for applying electrical arc spaced-apart electrodes disposed within a fluid-filled member to create flow and pressure waves.

### Description of the Related Art

A variety of techniques and instruments have been developed for use in the removal or repair of tissue in arteries and similar body passageways, including removal and/or cracking of calcified lesions within the passageway and/or formed within the wall defining the passageway. A frequent objective of such techniques and instruments is the removal of atherosclerotic plaque in a patient's arteries. Atherosclerosis is characterized by the buildup of fatty deposits (atheromas) in the intimal layer (i.e., under the endothelium) of a patient's blood vessels. Very often over time what initially is deposited as relatively soft, cholesterol-rich atheromatous material hardens into a calcified atherosclerotic plaque, often within the vessel wall. Such atheromas restrict the flow of blood, cause the vessel to be less compliant than normal, and therefore often are referred to as stenotic lesions or stenoses, the blocking material being referred to as stenotic material. If left untreated, such stenoses can cause angina, hypertension, myocardial infarction, strokes and the like.

Angioplasty, or balloon angioplasty, is an endovascular procedure to treat by widening narrowed or obstructed arteries or veins, typically to treat arterial atherosclerosis. A collapsed balloon is typically passed through a pre-positioned catheter and over a guide wire into the narrowed occlusion and then inflated to a fixed pressure. The balloon forces expansion of the occlusion within the vessel and the surrounding muscular wall until the occlusion yields from the radial force applied by the expanding balloon, opening up the blood vessel with a lumen inner diameter that is similar to the native vessel in the occlusion area and, thereby, improving blood flow.

Generally, known intravascular lithotripsy (IVL) devices include a voltage pulse generator in operative communication with one or more pairs of electrodes mounted on a catheter and within an inflatable balloon.

The angioplasty procedure presents some risks and complications, including but not limited to: arterial rupture or other damage to the vessel wall tissue from over-inflation of the balloon catheter, the use of an inappropriately large or stiff balloon, the presence of a calcified target vessel; and/or hematoma or pseudoaneurysm formation at the access site. Generally, the pressures produced by traditional balloon angioplasty systems is in the range of 10-15 atm, but pressures may at times be higher. As described above, the primary problem with known angioplasty systems and methods is that the occlusion yields over a relatively short time period at high stress and strain rate, often resulting in damage or dissection of the conduit, e.g., blood vessel, wall tissue.

IVL device durability is a complex design problem constrained by the balloon material, thickness, emitter construction, offsets between emitters and the balloon material, and the voltage pulses applied to the catheter that generate the IVL therapy. Current commercial IVL products demonstrate a declining pressure profile with each pulse of therapy delivered. This declining profile provides the most energy in the initial IVL pulses and declines with each pulse. Examples of this are illustrated in FIG. 36, which will be discussed in more detail below.

A portion of an exemplary known competitive IVL device is shown in cross-section in FIG. 2, viewed in cross-section along a line cutting through an IVL balloon which surrounds, inter alia, a catheter body. One manual measurement, using a destructive method to cut a section of the balloon to measure, using a calibrated micrometer has shown that one type of a known competitive IVL device has a single wall thickness of ^{˜}0.001″. Another measurement of a different known competitive IVL device yielded a single wall thickness result of ^{˜}0.0013″.

Thus, the prior art inflation port is in fluid communication with the fill lumen/passageway. The fill lumen/passageway of the competitive IVL device of FIG. 2 is formed between the inner surface of the catheter body and an outer surface of a sleeve that encompasses wire conductors (terminating at electrodes that are located along the catheter and within the balloon). Thus, the wire conductors of the known device are not exposed to the fluid within the fill lumen/passageway. Electrodes that are located within the balloon and electrically connected with the wire conductors are not covered by the encompassing sleeve and, therefore, are exposed to fluid within the balloon. In addition, the known competitive device comprises an angioplasty balloon that is adhered at both the proximal end of the balloon and the distal end of the balloon to an outer surface of the catheter body, with the fill lumen/passageway into the balloon's interior being formed within the catheter body and defined by a space between the sleeve and the inner surface of the catheter body. Accordingly, the competitive known IVL balloon is adhered at its proximal end and its distal end to one structure, that is, the outer surface of the catheter body. In addition, the competitive known IVL device of FIG. 2 comprises the catheter body, and the guidewire member (which defines the guidewire lumen) both extending through the balloon. Both of these structures continue distally beyond a distal end of the balloon to a distal tip.

Further, the sleeve shown in FIG. 2 adds a layer of material that adds crossing profile thickness, increases complexity and takes up a portion of the area of the fill lumen/passageway, thereby reducing the available volume fill lumen/passageway during inflation and/or deflation cycles.

FIG. 3 graphically illustrates the drop in impedance of a current leader across a spark gap defined between two spaced-apart electrodes in an IVL system following application of voltage to one of two spaced-apart electrodes as the current leader develops into an electrical arc between the spaced-apart electrodes. This, in turn, causes the power dissipated in the electrical arc to peak sharply while the voltage and current between the electrodes are both relatively high. The current reaches a peak and the voltage drops, both very rapidly, indicating that an electrical arc between the spaced-apart electrodes is present, or has occurred. The peak of the power dissipated in the electrical arc indicates the relatively short time interval during which all the useful work of heating the growing leader into an arc is performed. The graphic illustration of FIG. 3 is exemplary of one aspect of an IVL procedure that produces pressure waves.

Known IVL devices also comprise a spark gap between electrode pairs that, when a sufficiently high voltage is applied to a first electrode, facilitates a spark or electrical arc of current from the first electrode across the spark gap to a second electrode in the electrode pair. This process results in loss of material, or erosion, from each of the electrodes in the electrode pair. Known spark gaps are generally arranged axially, i.e., with terminal faces of wire conductors spaced apart from each other and facing each other in an axial spark gap configuration. In this case, the electrical arc involves the terminal or distal face of at least one electrode in the electrode pair. Continued arcing across the spark gap during an IVL procedure with these known devices results in erosion of material from each electrode involved in the electrical arc. Each of these arcs causes the spark gap to slightly increase in size and/or short, with the full procedural set of arcs resulting in an appreciable increase in spark gap size which may lead to unpredictability in generating an electrical arc, or reduced pressure. Alternatively, 1^{st} and 2^{nd} electrodes may be arranged concentrically, with the spark gap defined in a radial direction, wherein the 1^{st} and 2^{nd} electrodes are not arranged along a common radial or circumferential plane. As will be demonstrated herein, this arrangement generates pressure outputs with relatively high variability. In addition, known IVL devices produce a resultant pressure output, or “shockwave” or “pressure wave” that over a series of electrical arcs, decreases in magnitude as the IVL procedure is executed. Moreover, the highly variable pressure outputs produced by the known IVL devices can lead to unpredictable, or undesirable, outcomes and perhaps contribute to balloon instability over time due to the variability of the pressure outputs placing stress on the balloon material.

Further, known IVL coronary devices are configured to produce a total maximum number of 120 pulses per catheter at a frequency of 1 Hz.

The subject matter claimed herein is not limited to embodiments that solve any disadvantages or that operate only in environments such as those described above. Rather, this background is only provided to illustrate one exemplary technology area where some embodiments described herein may be practiced.

### BRIEF DESCRIPTION OF THE SEVERAL VIEWS OF THE DRAWINGS

These drawings are exemplary illustrations of certain embodiments and, as such, are not intended to limit the disclosure.

FIG. 1 illustrates a schematic intravascular lithotripsy (IVL) arrangement according to one or more embodiments of the present disclosure.

FIG. 2 illustrates a cutaway view of a known IVL device.

FIG. 3 illustrates a graphic illustration of a typical timing of applied voltage and current during application of voltage and generation of an electrical arc between spaced-apart electrodes.

FIG. 4 illustrates one embodiment of the present disclosure.

FIG. 5 illustrates one embodiment of the present disclosure.

FIG. 6 illustrates a side, cutaway view of a distal portion of an exemplary embodiment of the present disclosure.

FIG. 7A illustrates a side, cutaway view of an exemplary embodiment of the present disclosure.

FIG. 7B illustrates a side, cutaway view of an exemplary embodiment of the present disclosure.

FIG. 8 illustrates a side, cutaway view of a portion of the distal region of an exemplary embodiment of the present disclosure.

FIG. 9 illustrates a partial cutaway view of a portion of an exemplary embodiment of the present disclosure.

FIG. 10 illustrates a side, cutaway view of a portion of FIG. 6.

FIG. 11 illustrates a side, cutaway view of the embodiment of FIG. 10.

FIG. 12A illustrates an embodiment of a portion of a system of the present disclosure.

FIG. 12B illustrates an embodiment of a portion of a system of the present disclosure.

FIG. 12C illustrates an embodiment of a portion of a system of the present disclosure.

FIG. 12D illustrates an embodiment of a portion of a system of the present disclosure.

FIG. 13A illustrates an embodiment of a portion of a system of the present disclosure.

FIG. 13B illustrates an embodiment of a portion of a system of the present disclosure.

FIG. 14A illustrates a side, cutaway view of one embodiment of the present disclosure.

FIG. 14B illustrates a portion of device illustrated in FIG. 14A.

FIG. 15A illustrates one embodiment of the present disclosure.

FIG. 15B illustrates one embodiment of the present disclosure.

FIG. 15C illustrates one embodiment of the present disclosure.

FIG. 15D illustrates one embodiment of the present disclosure.

FIG. 15E illustrates a cross-sectional view of one embodiment of the present disclosure.

FIG. 16A illustrates one embodiment of the present disclosure.

FIG. 16B illustrates one embodiment of the present disclosure.

FIG. 16C illustrates one embodiment of the present disclosure.

FIG. 16D illustrates one embodiment of the present disclosure.

FIG. 17A illustrates one embodiment of the present disclosure.

FIG. 17B illustrates one embodiment of the present disclosure.

FIG. 17C illustrates one embodiment of the present disclosure.

FIG. 17D illustrates one embodiment of the present disclosure.

FIG. 18A illustrates one embodiment of the present disclosure.

FIG. 18B illustrates one embodiment of the present disclosure.

FIG. 19A illustrates one embodiment of the present disclosure.

FIG. 19B illustrates one embodiment of the present disclosure.

FIG. 20 illustrates a side view of one embodiment of the present disclosure.

FIG. 21 illustrates a schematic diagram of one embodiment of the present disclosure.

FIG. 22 illustrates a top cutaway view of one embodiment of the present disclosure.

FIG. 23 illustrates a block diagram of one embodiment of the present disclosure.

FIG. 24 illustrates a pressure plot comparing test and known IVL devices.

FIG. 25 illustrates a force tracking plot comparing tracking force through a tracking fixture for TEST and KNOWN IVL catheters.

FIG. 26 illustrates a flowchart for controlling and delivering voltage to electrodes and generating pressure waves according to one or more embodiments of the present disclosure.

FIG. 27A illustrates portions of circuitry arrangements of one or more of the IVL arrangement and control operation of FIGS. 1 and 26 according to one or more embodiments of the present disclosure.

FIG. 27B illustrates portions of circuitry arrangements of one or more of the IVL arrangement and control operation of FIGS. 1 and 26 according to one or more embodiments of the present disclosure.

FIG. 28A illustrates additional portions of circuitry arrangements of one or more of the IVL arrangement and control operation of FIGS. 1 and 26 according to one or more embodiments of the present disclosure.

FIG. 28B illustrates additional portions of circuitry arrangements of one or more of the IVL arrangement and control operation of FIGS. 1 and 26 according to one or more embodiments of the present disclosure.

FIG. 28C illustrates additional portions of circuitry arrangements of one or more of the IVL arrangement and control operation of FIGS. 1 and 26 according to one or more embodiments of the present disclosure.

FIG. 28D illustrates additional portions of circuitry arrangements of one or more of the IVL arrangement and control operation of FIGS. 1 and 26 according to one or more embodiments of the present disclosure.

FIG. 29 illustrates a flowchart for controlling and delivering voltage to electrodes and generating shock waves according to one or more embodiments of the present disclosure.

FIG. 30 illustrates a control operation flow according to one or more embodiments of the present disclosure

FIG. 31 illustrates graphic comparisons of voltages applied to a known IVL device and to embodiments of IVL devices according to the present disclosure.

FIG. 32 illustrates a graphic comparison of average peak pressure generated over 80 voltage pulses by a known IVL devices and embodiments of IVL devices according to the present disclosure.

FIG. 33 illustrates a graphic comparison of average peak pressure generated over 80 voltage pulses by a known IVL device and over a predetermined maximum number of voltage pulses by embodiments of IVL devices according to the present disclosure.

FIG. 34 illustrates an exemplary flowchart method according to the present disclosure.

FIG. 35 illustrates an exemplary flowchart according to the present disclosure.

FIG. 36 illustrates a plot of mean compressional pressure over voltage pulses for a competitive device.

FIG. 37 illustrates a plot of mean compressional pressure over voltage pulses for a a substantially flat pressure profile.

FIG. 38 illustrates a plot of mean compressional pressure over voltage pulses for a declining-flat pressure profile.

FIG. 39 illustrates a plot of mean compressional pressure over voltage pulses for a sawtooth declining pressure profile.

FIG. 40 illustrates a plot of mean compressional pressure over voltage pulses for an increasing pressure profile.

FIG. 41 illustrates a plot of mean compressional pressure over voltage pulses for an increasing-flat pressure profile.

FIG. 42 illustrates a plot of mean compressional pressure over voltage pulses for a sawtooth increasing pressure profile.

FIG. 43 illustrates a plot of mean compressional pressure over voltage pulses for a decreasing-flat-decreasing-flat pressure profile.

### DETAILED DESCRIPTION OF THE INVENTION

As discussed in additional detail herein, a refined approach to power characteristic control can assist in more effective and more consistent therapy. For example, improved durability, higher frequency and/or substantially equivalent pressure output, over a longer number of voltage pulses than previously possible, may be provided using embodiments of the present disclosure.

Traditional intravascular lithotripsy (IVL) devices, systems, and methods can apply high energy, electrical power to generate a spark (arc) between discharge electrodes. Under appropriate conditions, sparks generated by submerged electrodes can generate pressure waves within the medium which can be applied to treat (breakup) calcified lesions within the patient's vasculature. It can be appreciated that appropriate control of such high energy systems can be paramount to provision of effective and safe treatment.

Embodiments illustrated herein may include various physical configurations and/or pressure control algorithms which enhance balloon durability and/or enhance effective treatment as compared to previous IVS systems.

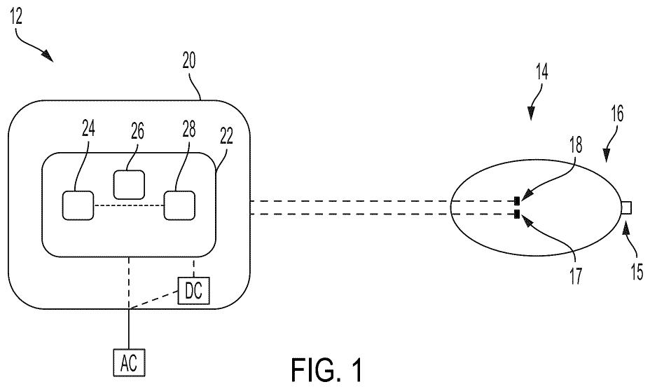

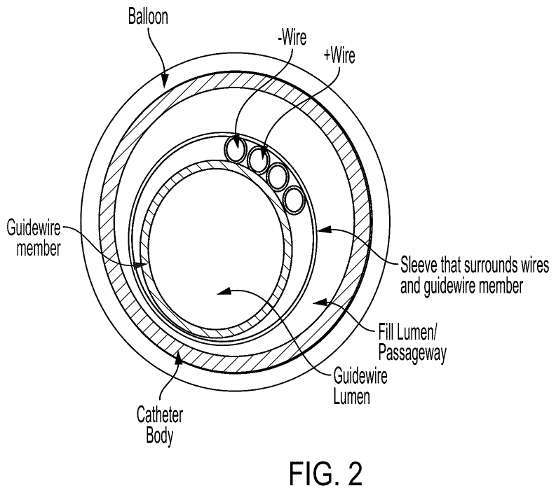

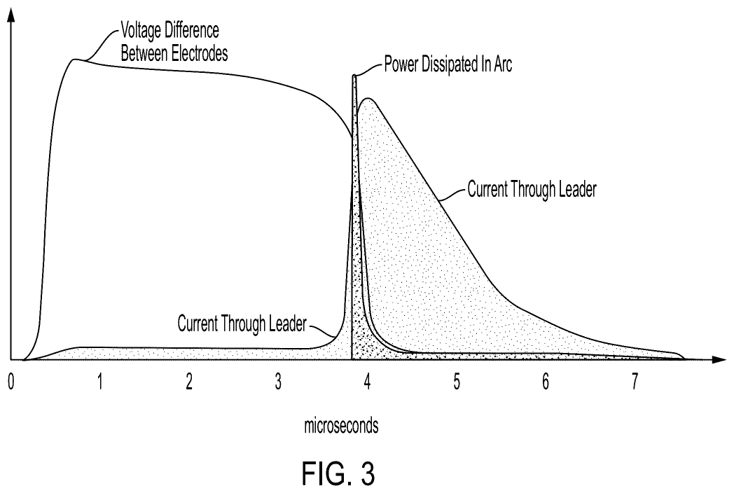

As illustrated in FIG. 1, a diagrammatic layout of portions of an exemplary IVL system 12 is provided. The illustrative IVL system 12 comprises a catheter assembly 14 including an elongate body, embodied as a catheter having guidewire 15, and a fluid-filled member 16 configured to contain conductive fluid therein, exemplified by an inflatable balloon, disposed near one end of the body and arranged to receive fluid for inflation to facilitate IVL therapy. A set of dischargeable spaced-apart electrodes 18 are shown arranged within the exemplary balloon, at least some of which are spaced apart by a gap 17 from each other to create a spark or electrical arc between the spaced-apart electrodes 18.

The IVL system embodiments described herein may be used in connection with electrodes that are within a fluid-filled member 16 configured to contain a fluid, e.g., a conductive fluid, therein. The fluid-filled member 16 embodiments may include an inflatable balloon as shown in FIG. 1, which may be compliant or non-compliant and serves to contain the fluid such that the spaced-apart electrodes 18 are submerged within the contained fluid. In addition, the fluid-filled member 16 may comprise a fillable member that is at least partially rigid and/or not flexible. In other embodiments, the fluid-filled member 16 may contain the fluid therein and wherein the spaced-apart electrodes 18 are located or submerged within the contained fluid.

Alternatively, the IVL system control embodiments of the present disclosure may be used in connection with electrodes that are not located or surrounded by a fluid-filled or fillable member 16. In these embodiments, the IVL system may comprise spaced-apart electrodes 18 that may be continuously or periodically exposed to saline or other fluid and, during the exposure, the IVL system may generate an electrical arc between the spaced-apart electrodes 18.

The spaced-apart electrodes 18 are arranged in communication (as suggested by dashed line conductors) with an electric pulse generation system 20 to receive high voltage electrical energy for spark generation to create pressure waves for IVL therapy. In the illustrative embodiment, one electrode may be grounded and the other provided with high voltage from the electric pulse generation system 20, although in some embodiments, any voltage differential may be applied. The electric pulse generation system 20 includes an IVL control system 22 comprising a processor 24 configured for executing instructions stored on memory 26 and communications signals via circuitry 28 for IVL operations according to the processor governance. The processor 24, memory 26, and circuitry 28 are arranged in communication with each other (as suggested via dashed lines) to facilitate disclosed operations.

Appropriate control of such high energy systems can also require achieving sufficient energy at the spaced apart electrodes. Given the high energy environment and microscale time periods for electronic discharge, desirable energy control within such IVL devices and systems can be challenging. Moreover, adaptable control methodologies may offer advantages to IVL effectiveness. Adjustable energy delivery can increase efficient power application, which can reduce risk to the patient. For example, beginning with a predetermined starting voltage threshold and defining a predetermined upper voltage threshold to form an acceptable voltage window may be proved. The acceptable voltage window may be coupled with series of generated voltage pulses of magnitudes that are confirmed to be within the acceptable voltage window. If, e.g., the magnitudes of the series of generated voltage pulses are below the predetermined upper voltage threshold, then the target voltage may be increased by a predetermined amount and another series of generated voltage pulses is executed.

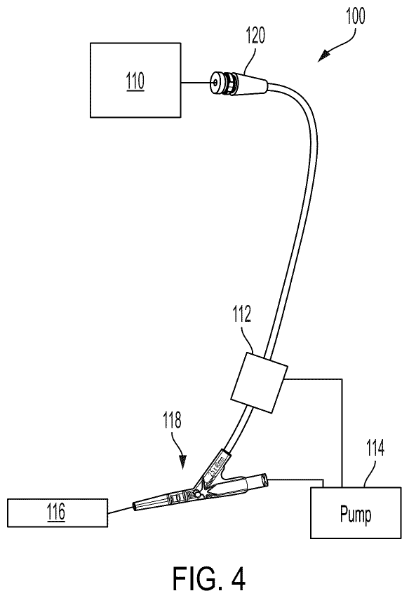

FIG. 4 illustrates an embodiment of an IVL system 100 of the present disclosure. A voltage pulse generator 110 is provided in operative connection and communication with a controller 112 configured to provide programmed operative instructions to the voltage pulse generator 110 and to a fluid reservoir/fluid pump device 114. The controller 112 and voltage pulse generator 110 are in operative electrical communication with wire conductors and pairs of electrodes as discussed above, wherein the wire conductors are disposed along the catheter's length and wherein the electrode pairs are disposed within the interior of an inflatable balloon that is positioned at or near a distal end of the catheter structure. The catheter and balloon structure are represented schematically by element number 116. A hub 118 allows operative connection and communication with the controller 112 and fluid reservoir/pump 114 and voltage pulse generator 110. In addition, connector 120 is in operative connection and communication with the controller 112 and provides operative electrical connection and communication with the wire conductors and electrode pairs. The controller 112 may comprise a processor for executing programmed instructions, for example initiating voltage pulses at a predetermined magnitude and frequency and in a predetermined pattern of pulses and magnitudes. The processor may be in operative communication and connection with a memory and a display. In some embodiments, the hub 118 may allow for over-the-wire guidewire access through a lumen defined within the catheter. In an embodiment, a rapid exchange (RX) access is provided. Some embodiments of the controller 112 may comprise an Electronically Programmable Read Only Memory (EPROM) comprising programmed instructions, for example and without limitation, initiating voltage pulses at a predetermined magnitude and frequency and in a predetermined pattern of pulses and magnitudes.

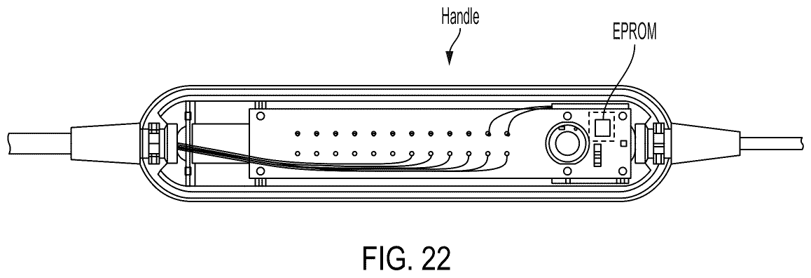

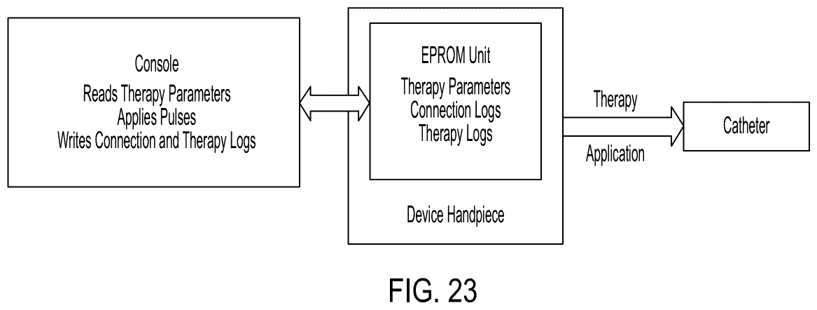

As discussed in FIGS. 22 and 23, some embodiments may comprise a handle may comprise an EPROM comprising programmed instructions, for example and without limitation, initiating voltage pulses at a predetermined magnitude and frequency and in a predetermined pattern of pulses and magnitudes. The EPROM may be in operative communication and connection with a console comprising a processor for executing programmed instructions, a memory in operative communication with the processor and, in some embodiments, the EPROM, and a display. In some embodiments, the hub 118 may allow for over-the-wire guidewire access through a lumen defined within the catheter.

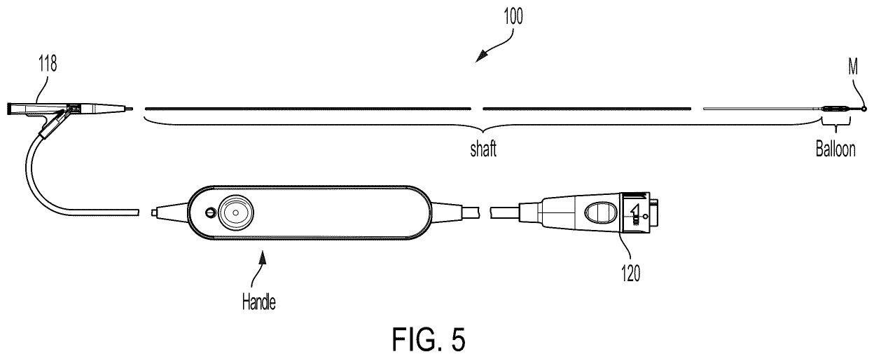

FIG. 5 shows a broken side view of the IVL system 100 comprising the handle with a connector 120 configured to connect with the voltage pulse generator 110 of FIG. 4 and a console as described above. A removable mandrel M is shown inserted through a guidewire lumen defined along a portion of the catheter shaft, extending proximally through a flexible distal tip and through the defined guidewire lumen of the catheter.

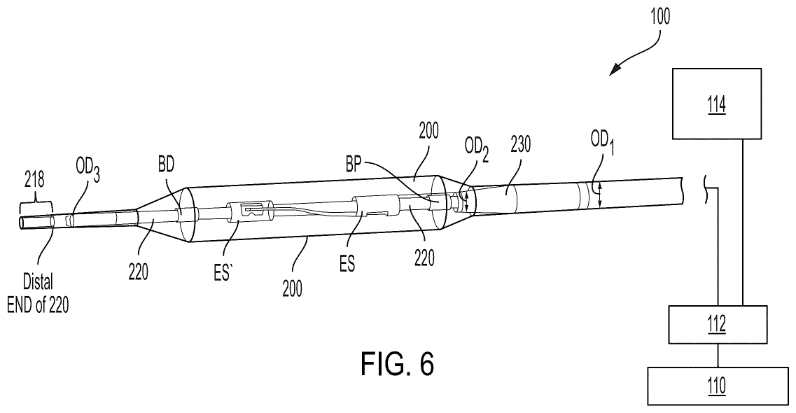

Distal portion embodiments of the catheter and balloon element 116 of the exemplary IVL system 100 of FIGS. 4 and 5 are illustrated in FIGS. 6-11.

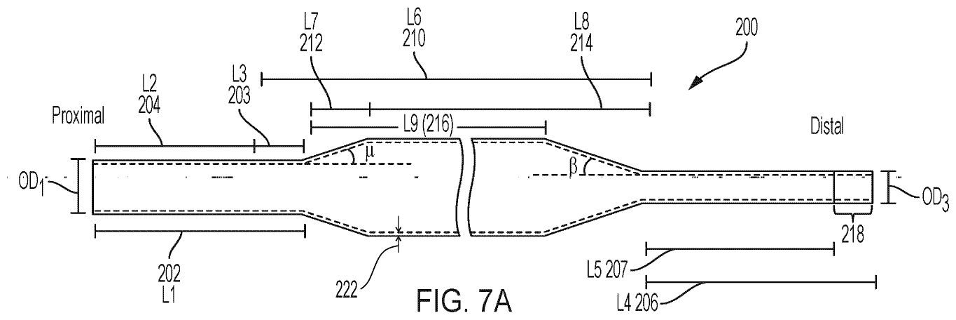

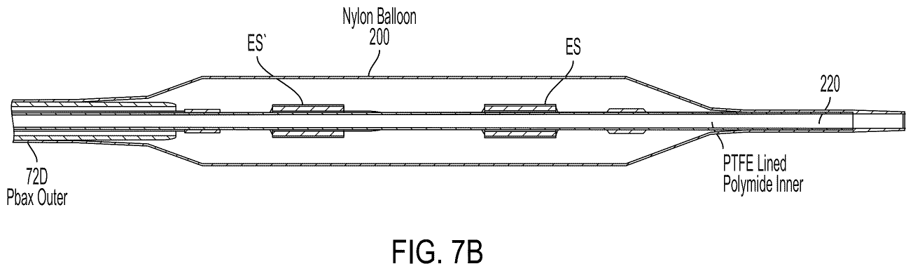

As best seen in FIG. 7 A, an inflatable balloon 200 is provided and comprises a cylindrical proximal section 202, a cylindrical distal section 206 and an inflatable portion 210 comprising an unbonded section 203 of the cylindrical distal section 206, a tapering proximal section 212, a tapering distal section 214 and a substantially cylindrical section 216 disposed between the tapering proximal and distal sections 212, 214.

A proximal portion 207 of the cylindrical distal section 206 of the balloon 200 surrounds, and is bonded or sealed in a watertight engagement against an outer surface of an elongate member 220 as best shown in FIG. 6. A distal region of the cylindrical distal section 206 extends, e.g., is extruded, beyond a distal end of the elongate member 220 to form an atraumatic, flexible tip 218.

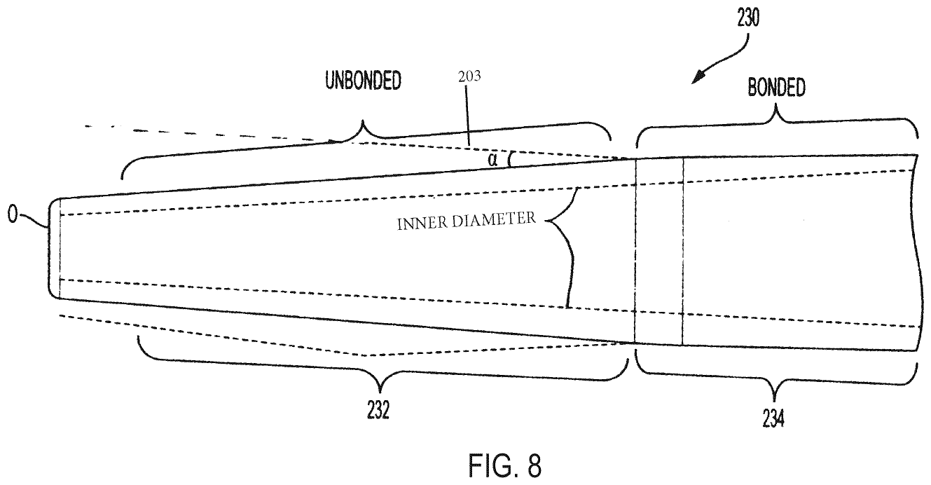

A proximal portion 204 of the cylindrical proximal section 202 of the balloon surrounds, and is sealed or bonded in a watertight engagement against, the non-tapering outer surface 234 of the tapering outer member 230 (see FIG. 8), and is not sealed or bonded against any portion of the elongate member 220 which is received within, and extends distally from, a distal end of the tapering outer member 230. A distal length comprising an unbonded section 203 of the cylindrical proximal section 202 of the balloon 200 surrounds the distal tapering section 232 of the tapering outer member 230, but is not bonded or sealed against any portion of the tapering outer member 230. As a result, the inflatable section of the balloon 200 comprises: the unbonded section 203 of the cylindrical proximal section 202, the proximal tapering section 212, the distal tapering section 214 and the substantially cylindrical section 216 disposed between the proximal and distal tapering sections 212, 214.

The tapering outer member 230 comprises an outer diameter that is larger than an outer diameter of the elongate member 220, and is configured to receive the elongate member 220.

In addition, a length of a fluid conveying pipe P is defined along the length of the device and between the concentric arrangement of the outer surface of the elongate member 220 and the inner surface of the tapering outer member 230. The remaining portion of the fluid conveying pipe P is defined proximally along the catheter shaft. The fluid conveying pipe P is provides the exclusive path for fluid communication between the fluid reservoir/pump 114 and the inflatable section 210 of the balloon 200. The fluid conveying pipe P terminates distally relative to the distal end of the tapering outer member 230 where an opening O is defined for fluid flow into, and out of, the balloon 200.

It is significant to note that the distal end of the tapering outer member 230, and therefore the opening O for fluid flow, extends distally beyond a distal end of the proximal cylindrical section 202 of the balloon. As a result, the proximal cylindrical section 202 of the balloon surrounds at least a portion of the proximal non-tapering, or cylindrical section, 234 of the tapering outer member 230, and is sealed or bonded against the non-tapering or cylindrical (proximal) section's 234 outer surface in a watertight seal. A distal end region of the tapering outer member 230 is, however, not surrounded by the proximal cylindrical section 202 of the balloon. Instead, the distal end region of the tapering outer member 230, and its opening O, extends into the proximal tapering section 212 of the balloon 200.

As a result of the watertight sealing mechanism described above, and the location of the opening O for fluid flow into and out of the inflatable portion 210 of the balloon 200, the proximal cylindrical section 202 of the balloon 200 plays no role in the movement of fluid into or out of the inflatable portion 210 of the balloon 200. Similarly, the inflatable portion 210 of the balloon 200 also plays no role in the inflation/deflation or degassing of the fluid, or the movement of fluid into or out of the inflatable portion 210 of the balloon 200. The inflatable portion 210 simply receives the incoming fluid from, or delivers outgoing fluid into, the opening O at the distal end of the tapering outer member 230.

FIG. 8 shows a distal portion of the tapering outer member 230, including a tapering section 232 comprising a tapering angle a. The tapering section 232 in this embodiment comprises the unbonded section 203 of the balloon 200, wherein the proximal cylindrical section 203 of balloon 200 (see FIG. 7 A) surrounds a portion of the tapering section 232, but is not adhered or bonded or sealed against the outer surface of the tapering section 232. Proximal of the tapering section 232 the tapering outer member 230 comprises a cylindrical section 234 of substantially constant diameter. This is indicated as the bonded section and provides an outer surface which the proximal portion 204 of the proximal cylindrical section 202 of the balloon 200 surrounds and is sealed or bonded in a watertight engagement. Sides of the fluid conveying pipe P is discussed above comprise a resulting inner diameter that is shown in dashed lines, wherein the pipe P terminates at an opening O at the distal end of the tapering outer member 230. As shown by the inner set of dashed lines, the inner diameter tapers distally within the tapering section 232. In other embodiments, the pipe P may be of substantially constant diameter through both the cylindrical section 234 and the tapering section 232, wherein the wall thickness of the pipe may neck down, or be made thinner moving in the distal direction at tapering section 232 while retaining a constant inner diameter through the cylindrical section 234 and the tapering section 232. In other embodiments, as in FIG. 8, the contours of the pipe P may taper downward with the tapering angle a of the tapering section 232. In some embodiments the inner diameter of the pipe P may comprise a constant diameter.

As shown in FIGS. 7 A and 7B, and with continued reference to FIGS. 6, 8 10 and 11, the cylindrical proximal section 202 of the balloon 200 comprises a length L1 and an outer diameter OD1. Even though the length L1 of the cylindrical proximal section 202 surrounds the tapering outer member 230, only a proximal portion 204 of the cylindrical proximal section 202 is bonded or sealed in a watertight configuration against an outer surface of the elongate member. The proximal portion 204, also referred to as the proximal watertight sealed or bonded portion 204 has a length L2 which is less than L1. Finally, the cylindrical proximal section 202 further comprises, on its distal end, an unbonded section 203 having a length of L3 and that surrounds the tapering outer member 230, but is not bonded or sealed to the elongate member, wherein length L3 is less than L2 and L1, such that L2 plus L3 equals L1.

The balloon 200 further comprises a cylindrical distal section 206 having an overall length of L4, including the distal tip 218. Without including the distal tip 218, the proximal portion 207 of the cylindrical distal section 206 that surrounds, and is sealed or bonded in a watertight engagement against the outer surface of, the elongate member 220 comprises a length of L5, which is shorter than L4. Accordingly, the distal tip 218 extends distally beyond the distal end of the elongate member 220 for a distance to provide an atraumatic tip and to facilitate translation through the vasculature.

As described above, the inflatable portion 210 of the balloon 200 comprises the unbonded section (length L3) 203, the tapering proximal section 212 and the tapering distal section 214, and the substantially cylindrical section 216 disposed therebetween. Accordingly, the length of the inflatable section is L6 which includes the unbonded section 203 of the proximal cylindrical section of length L3, the proximal tapering section 212 of length L7, the distal tapering section 214 of length L8, and the substantially cylindrical section 216 of length L9, for an overall inflatable section length of L6.

The tapering outer member 230 may taper down to a smallest outer diameter OD2 at its distal end that is smaller than outer diameter OD1 which is the effective outer diameter of the proximal cylindrical section 202 as well as the outer diameter of the non-tapering portion 234 of the tapering outer member 230. In this embodiment, an inner diameter of the balloon's cylindrical proximal section 202 may be substantially equivalent to the outer diameter of the tapering outer member at OD1, which is the non-tapering portion 234 of the tapering outer member to which the proximal cylindrical section is watertight bonded or sealed. Similarly, an inner diameter of the proximal portion 207 of the balloon's cylindrical distal section 206 that is watertight bonded or sealed to the elongate member 220 may be substantially equivalent to the outer diameter of the elongate member 220 at OD3.

As can be seen in FIGS. 6 and 7 A, the outer diameter of the outer member to which the balloon's cylindrical proximal section is sealed or bonded, that is OD1, may be greater than the outer diameter of the elongate member with outer diameter OD3.

As noted, the elongate member 220 is received within tapering outer member 230. As a result, the balloon 200 is sealed against two distinct structures. On the proximal side, the balloon 200 is sealed against the non-tapering outer surface 234 of the outer member 230 while on the distal side, the balloon 200 is sealed against the outer surface of the elongate member 220.

The balloon's distal tip 218 is preferably flexible and comprises a conduit defined therethrough and that aligns with a conduit defined through the elongate member to allow for, inter alia, guidewire access.

In some embodiments, the larger proximal side outer diameter OD1 as compared with smaller distal side outer diameter OD3, may produce a tapering angle μ for the tapered proximal section of the balloon that is different from, e.g., smaller than, the tapering angle β for the tapered distal section of the balloon. These tapering angles are measured with reference to the dashed lines of FIG. 7A, which are collinear with the non-tapering outer surface of the outer member (tapering angle μ, and the outer surface of the elongate member (tapering angle β). In some embodiments, the length L7 of the tapered proximal section may be shorter than the length L8 of the tapered distal section.

Because of these exemplary relative dimensions, as shown in both FIGS. 7 A and 7B, embodiments of the balloon 200 in the inflatable section 210 may be longitudinally asymmetric. In particular, the inflatable section 210 may be longitudinally asymmetric with a smaller tapering angle μ at the proximal tapering section 212 than the tapering angle β at the distal tapering section 214 of the balloon 200, which may help facilitate access into tight lesions. In other embodiments, the tapering angles μ and β may be substantially the same. In addition, embodiments of the distal cylindrical section 206 of the balloon 200 comprise a smaller outer diameter at OD3 than the proximal cylindrical section's 202 outer diameter, which may also facilitate access into tight lesions. Stated differently, the crossing profile of the device distal of the substantially cylindrical section 216 of the balloon 200 is smaller than the crossing profile of the device proximal of the substantially cylindrical section 216 of the balloon 200.

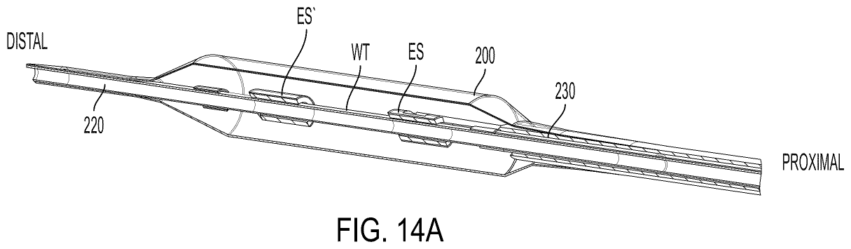

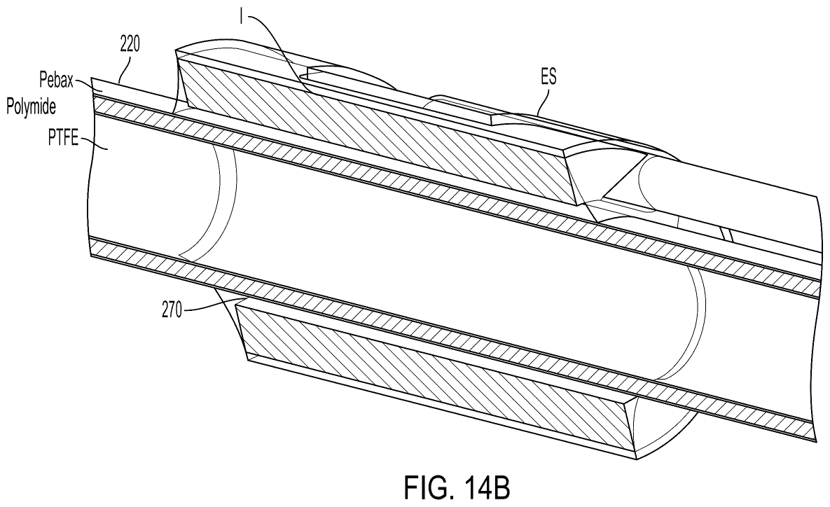

As shown in FIGS. 7B and 14B, the elongate member 220 to which the balloon 200 is sealed on the distal side comprises a polyimide core, which is lined on an inner surface with polytetrafluorethylene, commonly known as PTFE. The outer surface of the polyimide core is lined with 72D Pebax®. The electrode support members ES (proximal), ES' (distal) may be stainless steel and are coated with an insulating material such as a polymer or a blend of polymers or other materials, including but not limited to an adhesive, polyimide or other high temperature resistant flowable non conductive material.

Exemplary dimensions for the balloon 200 region may comprise the length of extension of the distal end of the outer member 230 into the inflatable section 210 a distance. An exemplary distance of extension of the outer member's distal end into the inflatable section is 0.794 mm, but other extension distances are within the scope of the present disclosure. The balloon may be sized and shaped for use in coronary vessels.

The balloon may be comprised of a polymeric material, such as Nylon, Vestamid, Polyamide, or similar material. In some embodiments, the balloon material is uncoated, which may allow for more efficient transfer of energy from the pressure waves therethrough.

In addition, the presence of the tapering outer member 230 which adds stiffness to the device, the unbonded section 203 of the balloon 200, the tapering section 232 of the tapering outer member 230 which provides for a smaller crossing profile in a wrapped balloon configuration, all function to provide additional pushability and strength in the region of the outer member, and further works to prevent kinking of the wrapped balloon device during advancement through a patient's vasculature, both of which are highly advantageous. As shown in FIGS. 6 and 9-11, a proximal marker band BP and a distal marker band BD may be provided within the balloon's inflatable portion 210 and disposed around the elongate member 220 at or near the transition from, respectively, the proximal and distal tapered sections 212, 214 into the substantially cylindrical section 216. In addition, a first proximal electrode support member ES is located along the elongate member 220 within the inflatable portion 210 at a position that may be closer to a proximal side of the balloon 200. In some embodiments, a single electrode support member ES may be provided as will be discussed further.

A second (distal) electrode support member ES' may be located along the elongate member 220 within the inflatable portion 210 at a position that is spaced distally from the first (proximal) electrode support member ES and closer to the distal side of the balloon 200. The two electrode support members ES and ES' are operatively and electrically connected by wire conductors W that are in operative electrical communication with the voltage pulse generator 10 as will be discussed further.

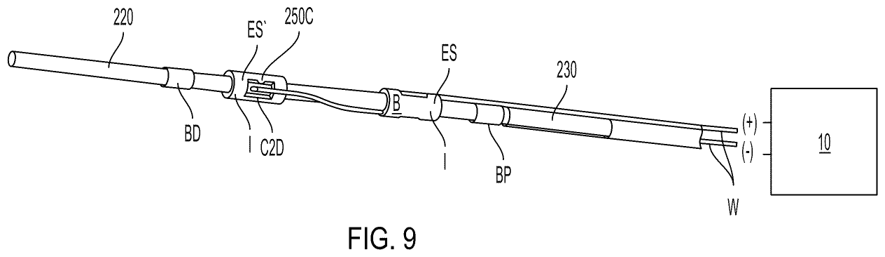

FIG. 9 illustrates a portion of the elongate member 220 and outer member 230 with the balloon 200 removed. Here, the first and second electrode support members ES, ES' are shown in closer detail with the wire conductors W connected in a series connection. Each electrode support member ES, ES' comprises a body B comprising a conductive material that is coated with an insulating material I and comprises at least one, preferably two, cutouts as will be discussed further. A tab or an arcuate region is defined on one of two longitudinal sides of each cutout and a wire conductor having an insulating covering with the exception of the distal-most end of the wire conductor which has no insulation As will be described further, a spark gap is formed between the exposed wire of the wire conductor and the tab or arcuate region of the cutouts of the electrode support members ES, ES′.

Each electrode support member may comprise two rotationally spaced cutouts with spark gaps which may be rotational spaced 180 degrees apart from each other, or may be spaced rotationally from each other at a different rotational spacing. As shown in FIG. 9, the spark gaps formed by the electrode support members ES and the spark gaps formed by the electrode support member ES' may also be rotationally spaced from each other. For example, if the spark gaps of the electrode support members ES and ES' are spaced 180 degrees apart around the respective electrode support member ES, ES′, then the two electrode support members ES and ES' may be radially rotated to ensure that all spark gaps are rotationally spaced apart from each other to provide circumferential coverage. In some embodiments, two or more cutouts and the respective spark gaps may be longitudinally aligned. As best shown in FIGS. 6 and 9, the electrode support members ES and ES' may be rotated relative to each other such that the respective cutouts and spark gaps are also rotationally spaced from each other around the elongate member 220. A rotational spacing may comprise a 90 degree rotational spacing between spark gaps along the elongate member 220, though other rotational spacings are within the scope of the present invention.

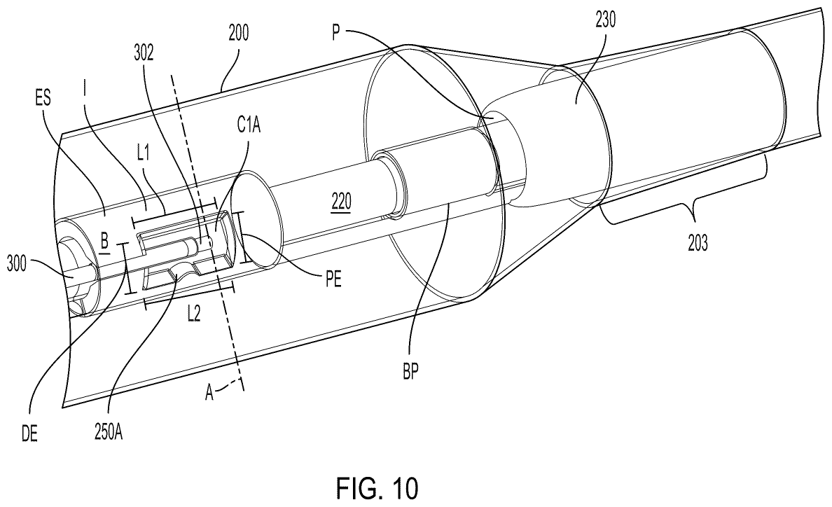

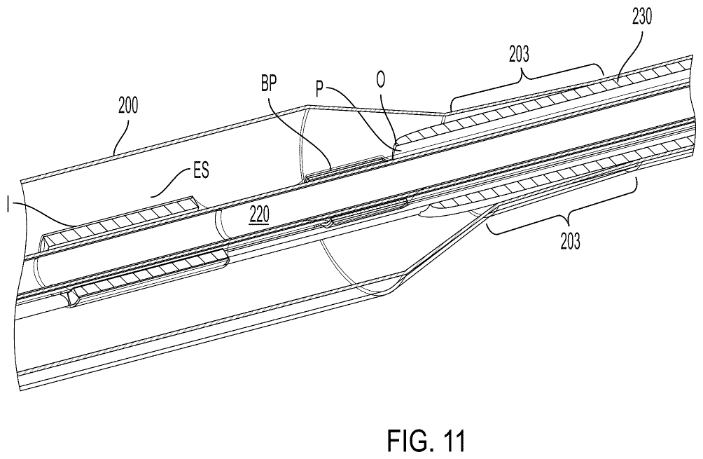

FIGS. 10 and 11 illustrate cutaway views of a proximal side of the balloon 200 and catheter structure, with a 1^{st} (proximal) electrode support member ES shown with an associated wire conductor W. In addition, the wire conductors leading back to the positive and negative terminals of the voltage pulse generator occupy the fluid conveying pipe.

FIG. 10 also illustrates a proximal-most 1^{st} electrode support member ES comprising a body B comprising a conductive material and defining a cutout C1A having two opposing longitudinal sides L1, L2 and opposing proximal and distal ends PE, DE. A tab or arcuate region 250 is formed or defined along one of the longitudinal sides L1. The surfaces of the electrode support body ES are covered in an insulating material I, with the exception of the tab or arcuate region 250, which comprises exposed conductive material.

As illustrated, a wire conductor 300 comprising insulation and having an exposed wire distal end region 302 that extends from a distal end a distance proximally. A proximal end region of the exposed wire region 302 is shown as located within the cutout, proximate to, and laterally or radially spaced from, the tab or arcuate region 250A. This configuration provides a pair of spaced-apart electrodes defining a spark gap between a lateral surface, and preferably not the distal end surface, of the exposed wire region 302 (comprising a first electrode in the illustrated spaced-apart pair of electrodes) and the tab or arcuate region 250 (comprising a second electrode in the spaced-apart pair of electrodes). In some embodiments, the distal end surface face of the wire conductor 300 may serve as an electrode in the above configuration.

An embodiment comprises the lateral surface of the exposed wire conductor and that is located at a distal region of the wire conductor, serving as one of the electrodes in a spaced-apart electrode pair. The illustrated embodiment of FIG. 10 comprises the lateral surface of the exposed wire region 302 of the wire conductor as a first electrode, meaning that current flows to this electrode first, then across the spark gap to the second electrode of the spaced-apart pair of electrodes. As will be discussed, this current flow may be reversed in certain embodiments of the spaced-apart electrodes, wherein the metallic region (in the FIG. 10, the embodiment is a tab or arcuate region 250A) comprises the first electrode in the spaced-apart electrode pair. In this embodiment, the lateral surface of the wire conductor comprises the second electrode in the pair of spaced-apart electrodes and current flows to the first exemplary tab or arcuate region 250A, then across the spark gap to the second electrode comprising the lateral surface of the exposed wire region 302.

An embodiment comprises the surface area of a first electrode and of a second electrode of a spaced-apart electrode pair to be substantially equivalent. In other embodiments, the surface area of a second electrode in a spaced-apart electrode pair may be larger than the surface area of a first electrode. Alternatively, the surface area of a first electrode in a spaced-apart electrode pair may be larger than the surface area of a second electrode.

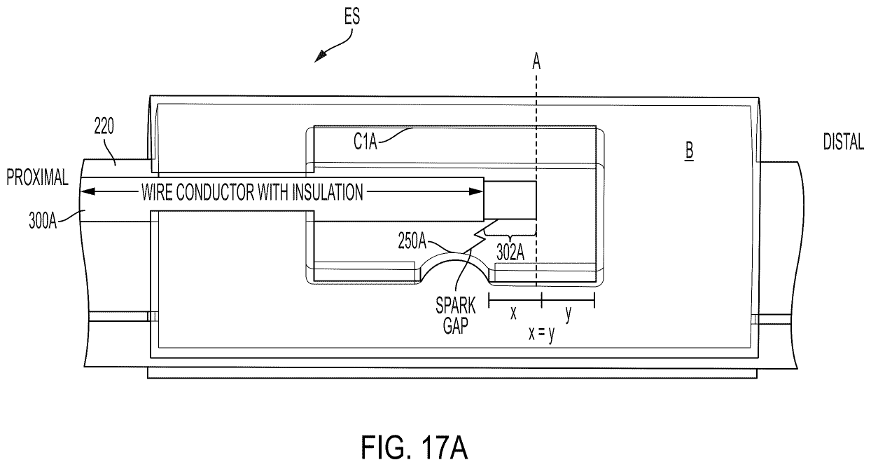

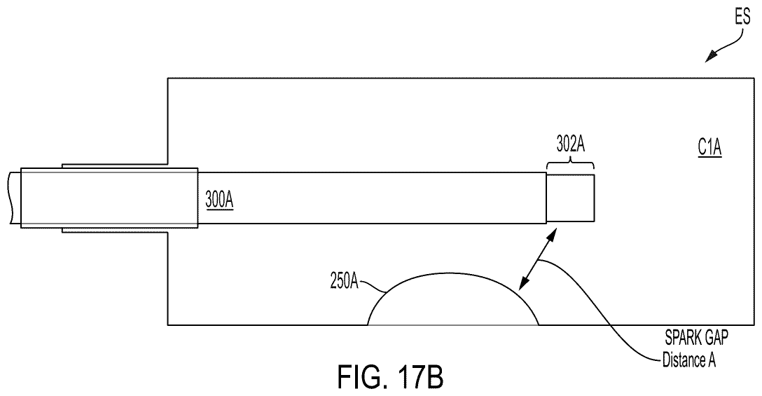

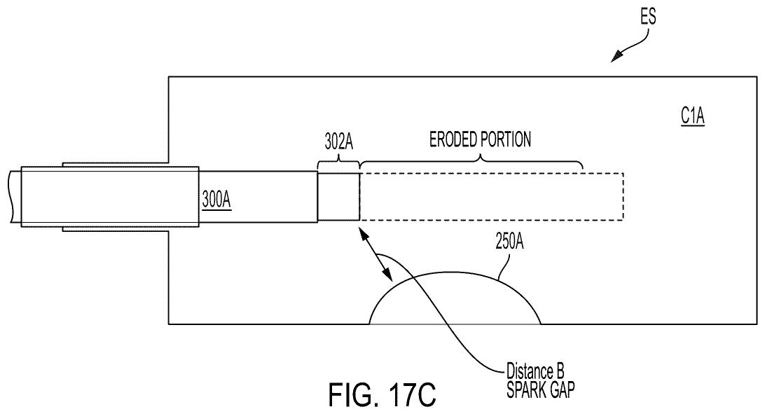

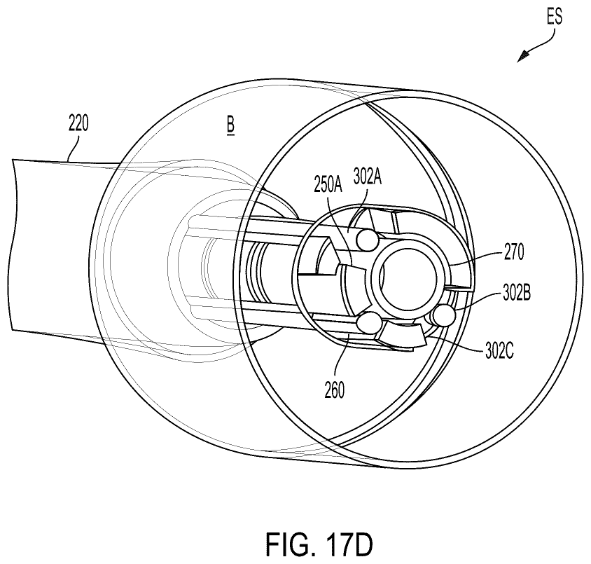

As will be discussed further, a location for the exposed wire region 302 is to position the distal end approximately one half of the distance between the proximal end of the tab or arcuate region 250A and the proximal end PE of the cutout C1A. That location is shown by axis A in FIGS. 10, 17A-C, 18 and 19. In this configuration, the portion of the wire conductor directly overlaying the tab or arcuate region 250 remains insulated, with the lateral surface of the exposed wire region 302 being positioned beyond (in this case proximally beyond) the bounds of the tab or arcuate region 250. The spaced-apart electrodes of the embodiments described herein are preferably located along a common radial or circumferential plane.

Though the tab or arcuate region (2^{nd} electrode) 250A is illustrated as positioned substantially mid-way along the length of the longitudinal side L1 of the cutout C1A, the tab or arcuate region 250A may be positioned more proximally or more distally as well. This changes the longitudinal position of the spaced-apart electrodes 250A, 302 as well as that of the defined spark gap between the spaced-apart electrodes 250A, 302, as well as effectively shifts the location and focus of the resultant pressure wave in the longitudinal direction to allow more effective coverage and/or interaction between adjacent generated pressure waves. FIG. 11 illustrates a cutaway of the region illustrated in FIG. 10.

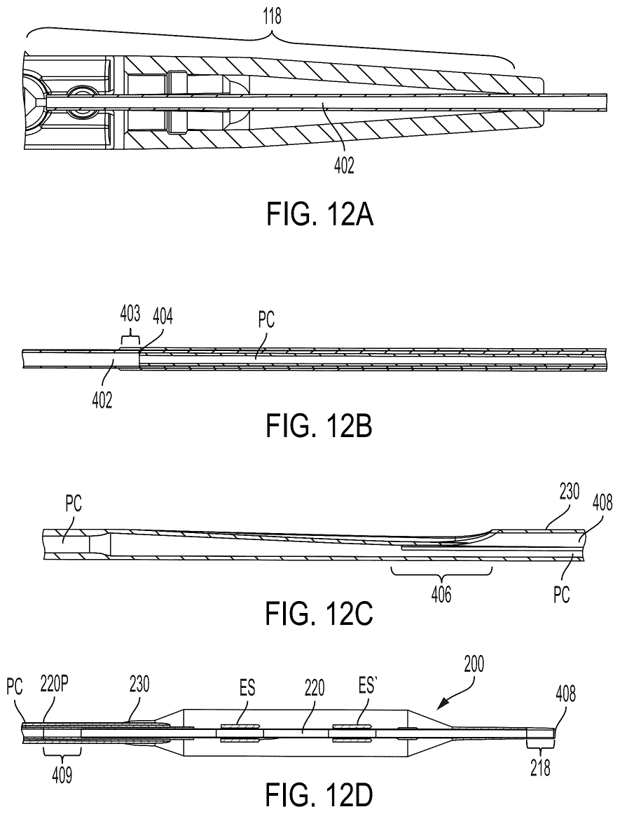

With continued reference to FIGS. 4-5 and 6-11, we now turn to FIGS. 12A-12D which illustrate features of an embodiment of an exemplary IVL system. FIGS. 12A-12D move successively distally along the exemplary system.

Beginning with FIG. 12A, just distal to the hub 118 as in FIG. 4, a hypotube 402 is provided which serves as a conduit for fluid infusion and removal and which is in fluid communication with the fluid reservoir and pump 114 as well as the interior of the inflatable portion 210 of the balloon 200. The hypotube 402 may be comprised of a metal and may be stainless steel. As shown in the combination of the structure shown in FIG. 12B, which is distal to the structure of FIG. 12A, an exemplary length of the hypotube 402 may be slightly longer than 1055 mm, though other lengths are within the scope of the present disclosure. FIG. 12B indicates a length of 1055 mm, however the hypotube 402 extends a slight distance further in the distal direction, along which it is overmolded by a 72D Pebax® material to form a bonded section 403 in order to, among other things, add bonding strength. The hypotube 402 is generally not coated on an outer or inner surface, but comprises a polymer near the distal end, e.g., Pebax® for aid in joining or bonding, and an adhesive on the proximal end region, also for aid in joining or bonding.

The hypotube 402 is shown as terminating distally at 404 in FIG. 12B. In some embodiments, the hypotube 402 may extend from the hub distally a distance of about 1080 mm. FIG. 12B continues a distance (e.g., and without limitation approximately 280 mm) distally from the bonded section 403 to comprise a polymide conduit PC for strain relief which is coated on its outer surface with the 72D Pebax®, which also comprises the bonded section 403.

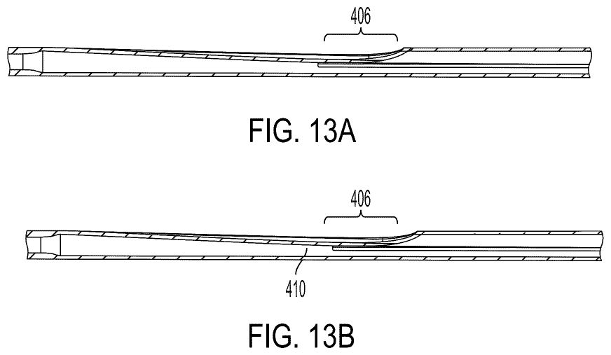

FIG. 12C illustrates the section comprising an RX port 406, which provides access for a guidewire or other interventional tools. The RX port 406 leads to a guidewire conduit 408 which may comprise a 63D Pebax® tube with a high density polyethylene (HDPE) such as Rezilok and having an inner surface that may be lined with 63D Pebax®. The guidewire conduit 408 extends distally through the polyimide conduit PC and the elongate member 230 and leads out of the system at the distal end of the distal tip 218. The RX port 406 is discussed further in FIGS. 13A and 13B. A proximal end of the outer member 230 comprising 63D Pebax® that is lined with HDPE, e.g., Rezilok, is provided just distal to the RX port 406 as shown in FIG. 12C. The outer member 230 continues for a length or distance distally to terminate at a distal end that is located within the inflatable section 216 of the balloon as shown and described above, as well as illustrated in FIG. 12D. The elongate member 220 comprises a proximal end 220P that connects with the polyimide conduit PC described above and comprising a bonded, e.g., laser reflowed, section 409 to aid in bonding. The elongate member 220 extends through the outer member 230 and the interior of the balloon 200 to a point that is just proximal to the distal tip 218.

Turning now to FIGS. 13A and 13B, the RX port 406 is illustrated. As shown a hypotube 410 is provided, which may comprise a polymer, along a length of the polyimide tube on the proximal side of the RX port 406 for support. With supplemental reference to FIGS. 14A and 14B discussed below, known devices typically employ a support wire instead of the hypotube 410, but the inventors found that the polyimide tube with an outer polymer jacket comprising the elongate member 220 that transitions into the hypotube 410 solution provides greater and necessary stiffness and support in this critical region.

FIG. 14A illustrates a cross-sectional view of the tapering outer member 230, balloon 200, elongate member 220 with 1^{st} and 2^{nd} electrode support members ES, ES′.

Electrode support members are in operative electrical communication with a voltage pulse generator 110 as discussed above. A bridge wire WT is provided for operative electrical connection between ES and ES′. The bridge wire may be made of tantalum rather than the known 1VL devices which use copper wire throughout. Tantalum provides significantly enhanced durability as compared with copper. During testing, copper wire used as a “bridge wire” would slowly deteriorate or erode as the test voltage pulses and associated electrical arcs and current flow progressed. Ultimately, the copper bridge wire became susceptible to becoming displaced and interrupting the series connection between electrode support members ES and ES′. Thus, tantalum bridge wires were found to provide key durability characteristics, one of the features of the present disclosure that allows fora far greater number of electrical arcs (up to, and greater than, 500) to be produced than the known devices.

FIG. 14B is a closeup of a cross-section of an electrode support member of FIG. 14A. The elongate member 220 may comprise 3 layers: a core of polyimide which aids in resistance of heat generated by the electrodes during operation, an outer layer of Pebax and an inner layer of PTFE. Other polymers or blends of polymers may be used in the construction of the elongate member 220.

In some embodiments, an air or fluid gap 270 is provided between the electrode support member, for example ES and/or ES′, and the outer surface of the elongate member 220 to which the electrode support member ES and/or ES' is adhered or operatively connected and at least partially surrounds. The air or fluid gap 270 functions to help to dissipate heat generated by the electrical arcs produced across the spark gap between the two spaced-apart electrodes 250, 302 described above, allowing the fluid within the inflated balloon 200 to flow through the air or fluid gap 270, and around and underneath a portion of the electrode support member ES and/or ES' to remove heat from the structure. This is another key durability element of the present disclosure and contributes to the far greater number of electrical arcs, and at a greater frequency of electrical arcs, that are produced with embodiments of this disclosure compared with the known devices.

Further, the electrode support members, e.g., ES, ES′, are coated with an insulating material, with the exception of the bare metal electrode element, e.g., tab or arcuate region 250, and the wire conductors defining an exposed wire electrode element are also otherwise coated with an insulating material. As a result, the surface area of the spaced-apart electrodes in each case is relatively tightly controlled. This is in contrast to a known IVL system which provides concentric metallic electrodes with much more exposed metal surface area than is actually required and, as a result, produce much more undesirable gas as a by-product of generating electrical arcs. This is another key feature of the present disclosure that contributes to durability as well as improved variability when compared with known IVL systems.

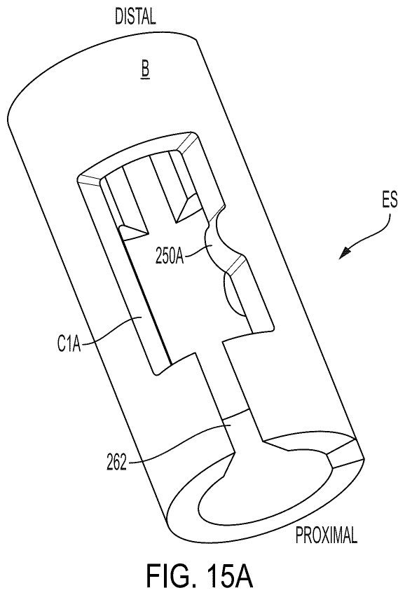

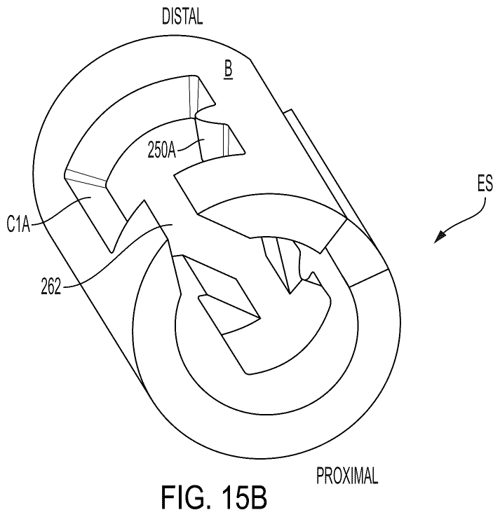

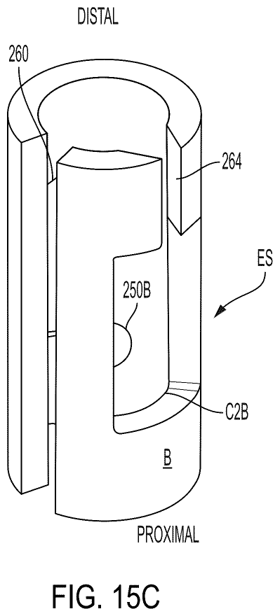

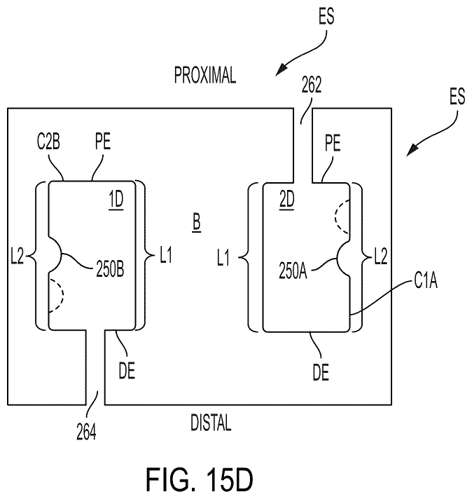

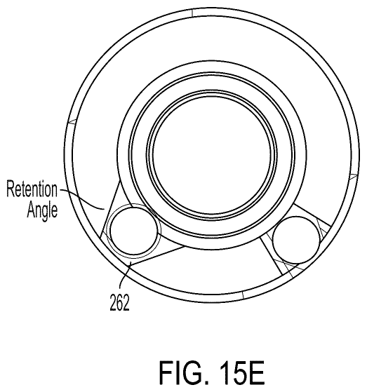

The operational connection or adherence of the electrode support members ES and/or ES' with the elongate member 220 in this embodiment is unique in that, as discussed above, the electrode support member ES and/or ES' is coated or covered with an insulating material I. The insulating material I flows beneath portions of the electrode support member ES and or ES' to form a connection or adherence between portions of the lower surface of the electrode support member ES and/or ES' and the outer surface of the elongate member 220, while retaining the desired air or fluid gap 270. Turning now to FIGS. 15A-15E, one embodiment of an electrode support member ES comprising two electrodes is illustrated. The electrode support member ES may be used in the IVL system embodiments described above. The electrode support member ES comprises a body B which may be cylindrical and configured to at least partially surround the elongate member 220 as discussed above. In some embodiments the electrode support members discussed herein may not be fully circumferential as will be discussed further. The embodiment of FIGS. 15A-D comprises two radially spaced-apart cutouts, a first cutout C1A and a second cutout C2B. Each cutout C1A, C2B comprises opposing longitudinal sides L1, L2 and a proximal end PE and a distal end DE. The body B also comprises a longitudinally arranged slot or channel 260 that extends all the way along the body and configured to receive a portion of an insulated wire conductor. The first cutout C1A comprises a slot or channel 262 extending longitudinally away in a proximal direction from the proximal end PE of the first cutout C1A. The second cutout C2B comprises a slot or channel 264 that extends longitudinally away in a distal direction from the distal end DE of the second cutout C2B.

The electrode support member ES comprises a body formed of a conductive material which is covered with an insulating material I as described above. A region along one of the opposing longitudinal sides comprises exposed conductive material, with the insulating material covering removed. In the illustrated embodiment of ES, the exposed conductive material, e.g., metal, is provided along longitudinal sides L2 at 250A and 250B, respectively, for each the first and second cutouts C1A, C2B. The embodiment of FIGS. 15A-15E provides the exposed conductive material portion as individual arcuate regions 250A, 250B that extend radially into each cutout C1A and C1B, respectively. Each of the exemplary arcuate regions 250A, 250B defines one electrode of a spaced-apart electrode pair.

As best seen in FIG. 15C, the longitudinal slot or channel 260 running the entire length of the electrode support member body B is provided and is configured to receive insulated portion of one or more wire conductors. FIG. 15E illustrates a cross-sectional view through body B showing that the longitudinal slot or channel 262 may comprise angled sides, with a smaller opening at the outermost portion of the slot or channel 262. This angled retention structure may be used to retain wires in slots 260, 262 and/or 264.

The slots or channels 260, 262, 264 are provided to maintain a crossing profile of the electrode support members that is, at maximum, the outer diameter of the electrode support member body. In addition, the slots or channels 260, 262, 264 function to retain the wire conductors and associated electrode regions in the proper position within the subject electrode support member.

FIG. 15D provides an “unrolled” flattened view of the exemplary electrode support member body B. The electrode-defining arcuate regions 250A, 250B of exposed conductive material may be substantially centered along the longitudinal side L1 or L2 defining the arcuate regions 250A, 250B. Alternatively, as shown by the dashed lines, one or both of the arcuate regions 250A, 250B of exposed conductive material may be shifted away from the center of the longitudinal side. Thus, the arcuate regions 250A, 250B may be centered along the subject longitudinal side and/or shifted away from the center, hi one embodiment, one of the arcuate regions, e.g., 250A may be offset longitudinally from the location of the other arcuate regions, e.g., 250B. This allows the locations of the arcuate regions 250A and 250B that are defined by a single electrode support member body B (and the resulting spaced-apart electrode, and defined spark gap locations) to be timed and, in some embodiments, offset both radially and longitudinally from each other. This, in turn, allows for generation of pressure waves by a single electrode support body B with two radially spaced-apart electrode pairs, wherein the pressure waves produce mechanical forces that are not only offset radially from each other, but also offset longitudinally from each other.

The formation of the spaced-apart electrode pairs, and defined spark gaps, using exemplary embodiment ES are generally described above, and will be further discussed below.

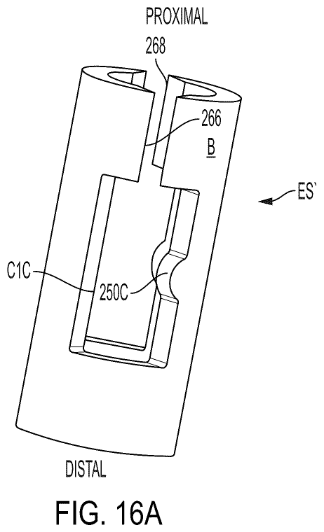

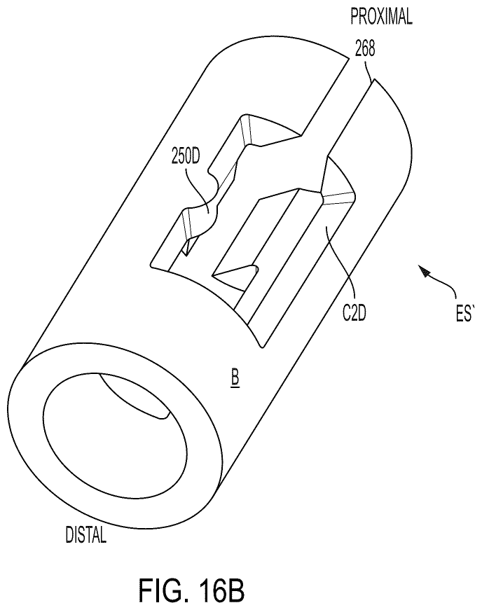

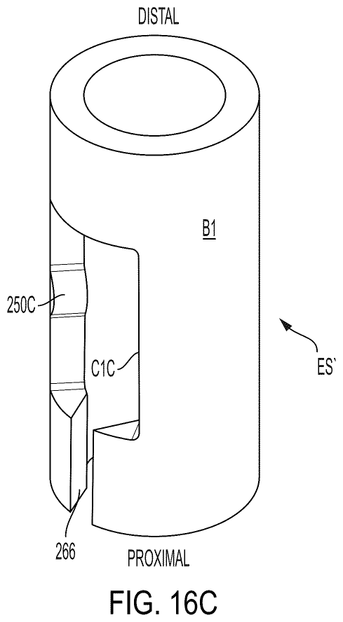

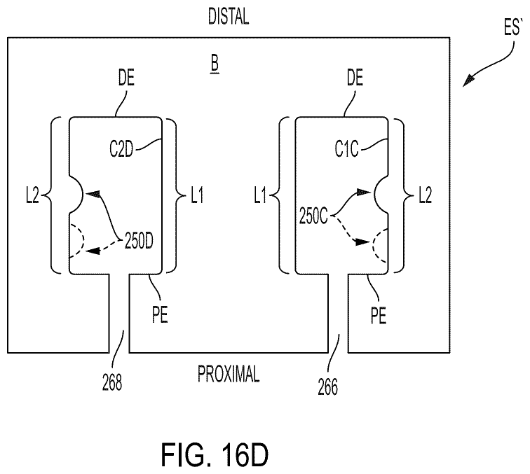

The exemplary electrode support member ES of FIGS. 15A-15E may comprise a proximal electrode support member such as shown in, e.g., FIG. 6 that is coupled with a more distally spaced-apart electrode support member ES' which is discussed below in FIGS. 16A-16D. In addition, when two or more of the electrode support members ES may be electrically and operatively connected with each other and with a more distally spaced-apart electrode support assembly, one of the electrode support members ES may comprise a proximal-most electrode support member and the remaining electrode support members ES may comprise intermediate electrode support members located between a proximal and a distal electrode support member.

A distal electrode support member ES' embodiment is illustrated in FIGS. 16A-16D. This embodiment may comprise a more distally located exemplary electrode support member ES′, such as shown in FIG. 6, when operatively combined with at least one more proximally-spaced apart electrode support member such as ES described above. More fundamentally and alternatively, this embodiment may be used alone, thus providing a single electrode support member ES' with two radially spaced-apart electrode pairs when fully assembled. In an alternate embodiment, the single electrode support member ES' may comprise a single pair of spaced-apart electrodes, wherein the spaced apart electrodes are in operative electrical communication with a first wire conductor that is in operative electrical communication with a first electrode of the electrode pair and a positive or high side terminal of a voltage pulse generator. A second wire conductor may be in operative electrical communication with a second electrode of the electrode pair and a ground or low side terminal of the voltage pulse generator.

The embodiment of FIGS. 16A-16D also comprises two radially spaced-apart cutouts, a first cutout C1C and a second cutout C2D. Each cutout C1C and C2D defines two opposing longitudinal sides L1, L2 and a proximal end PE and a distal end DE as well as a slot or channel 266, 268 extending in the proximal direction from the proximal end PE of each of the first and second cutouts C1C and C2D. As with the embodiment of FIGS. 15A-15E, exemplary arcuate regions of exposed conductive material 250C and 250D are defined along one of the longitudinal sides of cutouts C1C and C2D, respectively. As best seen in FIG. 16D, the regions of exposed conductive material, e.g., metal, 250C and 250D are each defined along longitudinal side L2 of the respective cutout C1C and C2D. Each arcuate region 250C and 250D forms and defines one electrode of a space-apart pair of electrodes. As with the embodiment of FIGS. 15A-15E, the location of one or both of the arcuate regions 250C and 250D (and the defined spark gap when the wire conductors are added, and the location of the pressure wave produced by the resultant spaced-apart electrodes) may be longitudinally shifted from the center of the defining longitudinal side(s) as shown with the dashed lines in FIG. 16D. This embodiment may not comprise the full length longitudinal slot 260 of the embodiment of FIGS. 15A-15E.

An exemplary spaced-apart electrode pair is illustrated in FIGS. 17A-17D. An exemplary electrode support member, which may be either of the embodiments discussed above in FIGS. 15A-16D, is provided. We will describe the illustrated embodiment as an electrode support member ES as described above in connection with FIGS. 14A-14D. A first cutout C1A is shown illustrated with a portion of an insulated wire conductor 300 A located or received within the slot or channel 262 extending proximally away from the first cutout C1A. A distal-most region 302 A of the wire conductor 300A is stripped of insulation, leaving an exposed distal-most region 302A of exposed conductive wire. Together with the exposed metal of the arcuate region 250A, the lateral surface, as opposed to the distal end face or surface, of the exposed distal most region 302a of the wire conductor 300A forms a spaced-apart electrode pair, with a spark gap defined therebetween.

The location of the exposed conductive wire region 302 A is preferably positioned beyond the arcuate region as shown such that a distal end of the exposed wire region is halfway between the arcuate region and the distal end of the cutout C1A. This dimensioning is illustrated by x and y, wherein x=y in FIG. 17 A. Alternatively, the exposed wire region 302A at the distal end of the wire conductor may be positioned generally over the arcuate region to form an alternative embodiment of a spaced-apart electrode pair.

FIGS. 17B and 17C illustrate an exemplary starting and ending positions for the electrode comprising the exposed wire region 302 A relative to the spaced-apart electrode comprising the exemplary arcuate region 250A. FIGS. 17B and C also illustrate a general direction of the current flow and electrical arc across the spark gap defined by the spaced-apart electrode pairs with distance A representing a starting spark gap length and distance B an ending spark gap length between the lateral face of the exposed wire conductor (exemplary first electrode) and the exposed conductive material of the arcuate region (exemplary second electrode). The current flow and resulting electrical arc will translate axially along with the translating exposed wire region 302A.

As the electrical arcing initiates and progresses between the spaced-apart electrodes 302 A and 250 A, the electrode comprising the exposed wire region 302 A begins to erode and translate, effectively move axially (in the illustrative embodiment proximally) along the arcuate region 250A and engaging successively different (more proximal) regions of the arcuate region 250A in the electrical arcing process. The insulation initially covering the wire conductor 300A burns away, exposing successively more wire conductor and, as in FIG. 17C, with an exemplary ending position with a spark gap of distance B. As is now apparent, the spark gap changes relative locations, moving in this case in the proximal direction along a longitudinal axis of the wire conductor 300A. As the spark gap location changes, so does the effective direction of the current flow and resultant electrical arcs.

FIG. 17D illustrates a cross-sectional view through an exemplary electrode support member ES described in connection with FIGS. 15A-15E to show the relative positions and locations of exemplary first electrode comprising the exposed wire region 302 A an exemplary second electrode comprising arcuate region 250A to define a first spaced-apart electrode pair. In addition, the relative positions and locations are shown for a second spaced-apart electrode pair, circumferentially or radially spaced away from the first spaced-apart electrode pair. The second spaced-apart electrode pair includes an exemplary second electrode comprising the exposed wire region 302B and an exemplary second electrode comprising arcuate region 250B. It is preferable that, in all embodiments described herein, the electrodes formed by the lateral face of an exemplary exposed conductive wire 302 A, 302B and the respective and exemplary exposed metal region, illustrated as arcuate region 250A, B, be located at positions that are substantially the same relative to the outer surface of the elongate member 220. Stated differently, the spaced-apart electrodes formed by 302A, 250 A and 302B, 250B may each be located a distance from the outer surface of the elongate member 220, wherein the distance from a spaced apart electrode 302 A and 302B to the outer surface of the elongate member 220, and the distance from spaced-apart electrode 250A and 250B to the outer surface of the elongate member 220 are substantially equal. This arrangement provides an air or fluid gap 270 which is discussed further above. Moreover, with reference to FIGS. 15A-15E, at least part of the lateral surface of the exposed wire section of the first wire conductor may located between, and aligned with, the first and second longitudinal sides L1, L2 of the cutout such that the lateral surface of the exposed wire section is spaced apart from, and aligned with, the exposed metal region. Alternatively, the spaced-apart electrodes formed by 302 A and 250A may each be located along the outer surface of the elongate member 220.

In all embodiments, the spaced-apart electrodes of the present disclosure are provided at substantially equal distances from a longitudinal axis through the IVL device's shaft.

In some embodiments, the spark gap represented by distances A and B may be of equivalent length. This is significant as it allows for use of a predictable and predetermined voltage magnitude to be provided which, in turn, produces a much more controlled pressure output from the produced pressure waves. Ultimately, the pressure output produced with a controlled, known spark gap length comprises more consistent, less variable forces than known IVL devices. This is understood to be critical in producing less strain on the balloon which, in turn, allows for a larger number of maximum voltage pulses, electrical arcs and produced pressure waves with a single catheter or system than is currently possible. For example, a known IVL coronary device has a maximum of 120 voltage pulses. The disclosed embodiment is demonstrated to effectively produce 300 voltage pulses per catheter in some embodiments and up to 500 pulses per catheter in some embodiments, with associated produced pressure waves, all within a very tight distribution and without significant decrease in the produced pressure outputs across the 300 voltage pulses. In some embodiments, the spark gap may be controlled such that it is within a predetermined range of length, with an exemplary minimum spark gap of 0.004″.

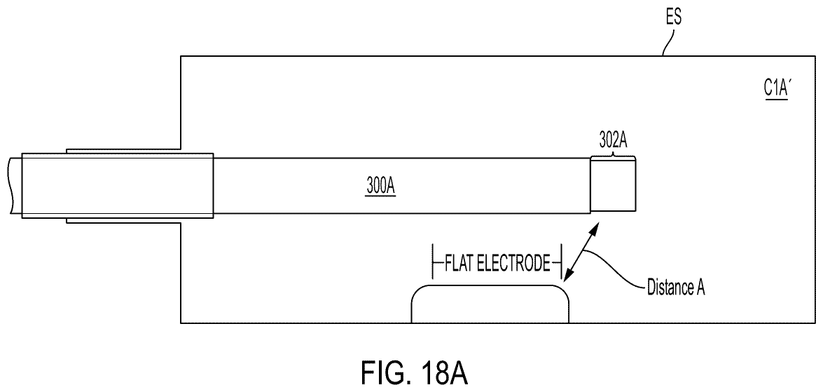

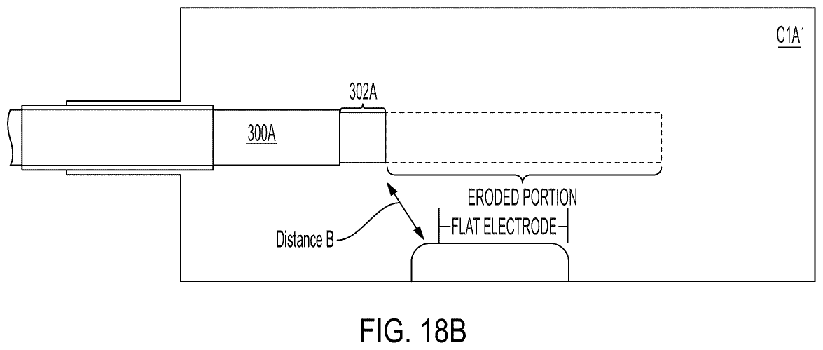

FIGS. 18A and 18B are similar to the embodiment of FIGS. 17A and 17B, wherein the arcuate region electrode is replaced with a cutout C1A′ comprising a raised flattened region that comprises exposed metal and functions as an electrode. This arrangement further ensures that, as the lateral surface of the exposed wire region 302 A is involved in electrical arcs with the raised flattened region, the spark gap distance remains substantially the same across the executed series of voltage pulses and electrical arcs. Thus, distances A and B are substantially equal, and each spark gap therebetween is also substantially the same length as distances A and B.

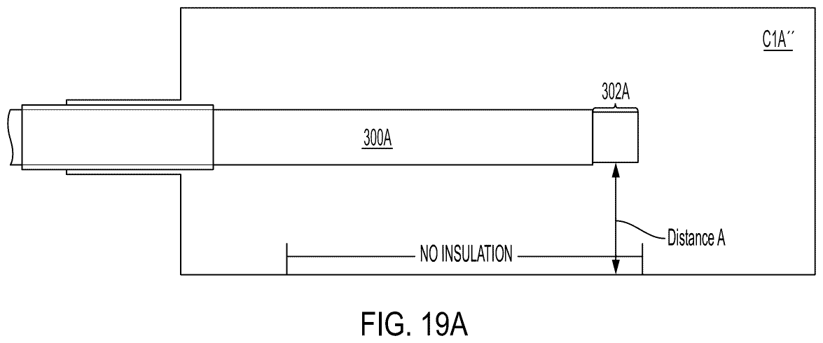

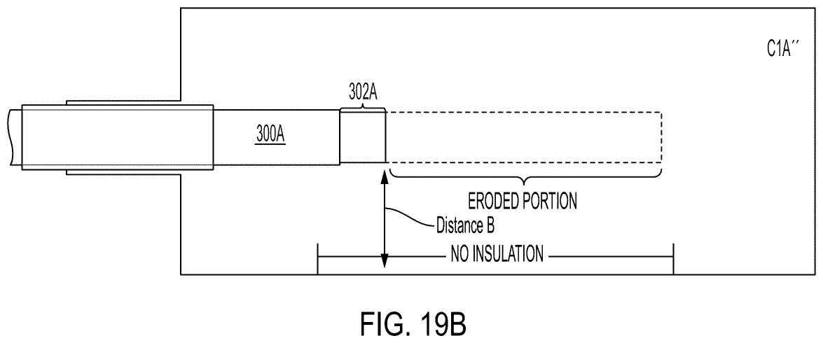

FIGS. 19A and 19B are alternative embodiments, similar in function with FIGS. 18A-18B except that the raised flattened region electrode is replaced with an inner surface of the cutout C1A″ of the electrode support member body B that is stripped of insulation for a length or distance, wherein the region of exposed conductive material functions as an electrode. This arrangement further ensures that, as the lateral surface of the exposed wire region 302 is involved in electrical arcs with the electrode comprising exposed conductive material, the spark gap distance remains substantially the same across the executed series of voltage pulses and electrical arcs. Thus, distances A and B are substantially equal, and each spark gap therebetween is also substantially the same length as distances A and B.

In some embodiments, the spark gap shaping and related distance as the erosion of the wire conductor's exposed wire lateral surface erodes may be timed to the magnitude of the voltage pulses generated by the voltage pulse generator. In such embodiments, an initial series of voltage pulses at a predetermined magnitude, tuned to ensure that electrical arcs are produced between the spaced-apart electrodes at the known spark gap distance. In some embodiments, the spark gap distance may change at a known rate and within one or more of a plurality of series of voltage pulses and associated arcing as the electrical arcing process is executed. Thus, as the erosion process progresses, the lateral surface of the wire conductor's exposed wire is engaged in arcing and the exposed wire region, e.g. 302A, begins to translate and traverse over the electrode (exposed metal) surface of the electrode support member. During this shortening traversal, the spark gap distances may be substantially the same for a period of pulses/arcs and/or may change across the executed pulses/arcs. Changing spark gap distances may be correlated with the known spark gap distances relating to the relative positions of the lateral surface of the exposed wire of the wire conductor and the location range of produced electrical arc engagement along the exposed conductive material region of the electrode support member that comprises an electrode.

Thus, the controller may correlate the required, or target, voltage pulse magnitude with the known spark gap distances, or distances ranges, for an initial series of voltage pulses and associated electrical arcs and further through to the maximum allowed number of voltage pulses and/or electrical arcs for a specific device. The known spark gap distances over time and produced arcs, allows the controller to modify the voltage magnitude as the pulse numbers progress (and the spark gap distances change) in order to ensure (1) that an electrical arc occurs; and/or (2) that the pressure output resulting from the controller-initiated voltage pulse at a predetermined magnitude is within a relatively tight and controlled window. In some embodiments, the controller may determine whether sufficient electrical energy was released by an energy storage element to produce an electrical arc.

The pairs of spaced-apart spark gaps may be arranged, and wired, in a variety of ways.

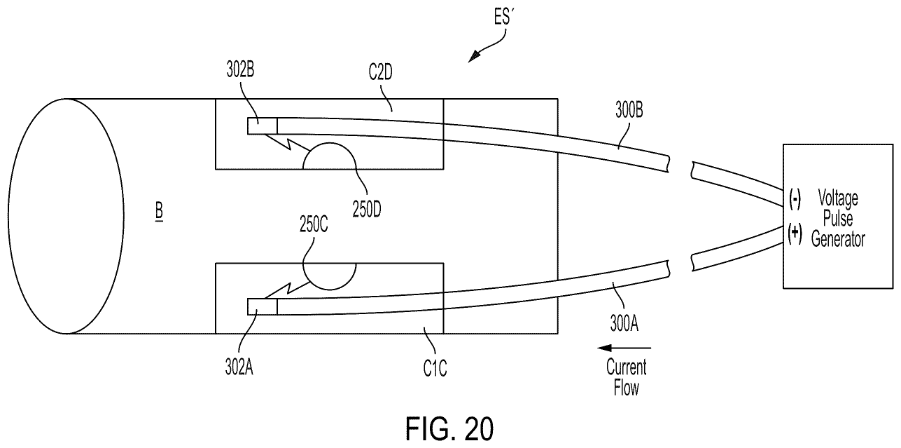

Perhaps the simplest arrangement involves the electrode support member ES' discussed above in connection with FIGS. 16A-16D. As shown in FIG. 20, the electrode support member ES' comprises a first cutout C1C with a first wire conductor 300 A received within slot or channel 266 and with a first exposed wire 302 A having a lateral face serving as a first electrode and positioned in a spaced-apart location relative to an exemplary exposed metal arcuate region 250C serving as a second electrode in a first spaced-apart electrode pair.

As discussed in connection with FIG. 17D, it is preferable that the spaced-apart electrodes in all embodiments described herein be located at positions that are substantially the same relative to the outer surface of the elongate member 220. Stated differently, the spaced-apart electrodes may each be located a distance from the outer surface of the elongate member 220, wherein the distance from a first spaced apart electrode in the electrode pair to the outer surface of the elongate member 220 is substantially equal to the distance from a second spaced-apart electrode of the electrode pair to the outer surface of the elongate member 220. Moreover, at least part of the lateral surface of the exposed wire section of the first wire conductor may be located between the first and second longitudinal sides of the cutout, and aligned with lateral surfaces of the first and second longitudinal sides of the cutout and with the exposed metal region. Alternatively, the two spaced-apart electrodes of an electrode pair may each be located along the outer surface of the elongate member 220.

The second cutout C2D also comprises an exposed metal arcuate region 250D which serves as a third electrode. Second cutout C2D also receives a second wire conductor

300B within slot or channel 268 and wherein a distal end of the second wire conductor 300B comprises an exposed wire region 302B with a lateral face thereof serving as a fourth electrode.

In operation, when the voltage generator initiates a voltage pulse of sufficient magnitude, current will flow through the first wire conductor 300 A to the first electrode 302 A and across a first spark gap from the first electrode 302A to the second electrode 250C within the first cutout C1C, creating an electrical arc and resultant pressure wave. Current will continue to flow through the conductive body B of the electrode support member ES' until reaching the third electrode at exemplary arcuate region 250D of the second cutout C2D. Current will flow across a second spark gap from the third electrode to a fourth electrode which comprises the second wire conductor 300B and its distal region of exposed wire 302B, with the current then flowing along second wire conductor 300B back to a negative terminal of the voltage pulse generator. As the current flows across the second spark gap from the arcuate region 250D to the distal region of exposed wire 302B in the second cutout C2D, an electrical arc is generated with resultant pressure wave. The second wire conductor is in operative electrical communication with a negative or ground terminal of the voltage generator and the first wire conductor is in operative electrical communication with a positive or high side terminal of the voltage generator.

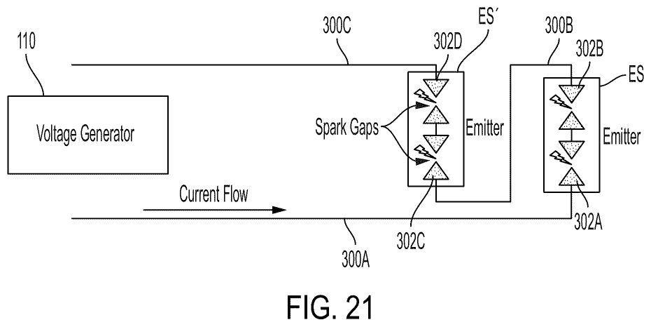

FIG. 21 illustrates two electrode support members connected in a series connection. With continued reference to FIGS. 15A-15E, the more proximally positioned first electrode support member may comprise electrode support member ES which, as discussed, comprises two radially spaced-apart cutouts C1A, C1B, each cutout C1A, C1B defining a pair of spaced-apart electrodes with a spark gap therebetween as described herein. The second (more distal) electrode support member may comprise ES' as described above and, like ES, comprises two radially spaced-apart cutouts C2C, C2D, each cutout C2C, C2D defining a pair of spaced-apart electrodes with a spark gap therebetween.

With continued reference to FIGS. 15A-16D, the current flow in FIG. 21 initiates at the voltage generator producing a voltage pulse of sufficient magnitude. The resulting current flows distally along a first wire conductor 300A to reach a first electrode comprising the first wire conductor's distal region of exposed wire 302A. The lateral face of the exposed wire region 302A is in spaced-apart relation with a first cutout's C1A arcuate region 250A which serves as a second electrode in the spaced-apart electrode pair as described above. As described above in connection with FIG. 17D, it is preferable that the spaced-apart electrodes in all embodiments described herein be located at positions that are substantially the same relative to the outer surface of the elongate member 220. Stated differently, the spaced-apart electrodes may each be located a distance from the outer surface of the elongate member 220, wherein the distance from a first spaced apart electrode in the electrode pair to the outer surface of the elongate member 220 is substantially equal to the distance from a second spaced-apart electrode of the electrode pair to the outer surface of the elongate member 220. Moreover, at least part of the lateral surface of the exposed wire section of the first wire conductor may located between the first and second longitudinal sides of the cutout and aligned with the exposed metal region. Alternatively, the two spaced-apart electrodes of an electrode pair may each be located along the outer surface of the elongate member 220.

The current flowing across this first spark gap will create an electrical arc and associated pressure wave. Continuing with reference to FIGS. 15A-16D and FIG. 21, current will continue to flow through the conductive material of the body B of the more proximally located electrical support member ES until reaching a third electrode comprising a second arcuate region 250B within a second cutout C2B that is radially spaced from the first cutout C2A. A second wire conductor 300B or bridge wire comprising tantalum provides a proximal and a distal end regions wherein both end regions comprise exposed tantalum wire, with the remaining tantalum wire covered in insulation. The proximal end of the exposed tantalum wire comprises a lateral face at 302B functions as the fourth electrode in this system and which is preferably in spaced-apart relation with the third electrode defined by the second arcuate region 250B.

As the current flows from the third electrode to the fourth electrode within the second cutout C2B, a second electrical arc is formed across the spark gap and a pressure wave results.

Next, the current flows along the second wire conductor 300B comprising the tantalum bridge wire to a more distally spaced and located electrode support member ES′. The distal exposed wire 302C of the tantalum bridge wire comprises a lateral face that serves as a fifth electrode in this system and which is located within a first cutout C 1C of the electrode support member ES′. The lateral face of the fifth electrode is spaced apart from a sixth electrode comprising an arcuate region 250C of exposed metal of the first cutout C1C of the electrode support member ES′. As current flows from the fifth electrode across the defined spark gap to the sixth electrode, an electrical arc is generated and a pressure wave is produced.

Current continues to flow from the sixth electrode through the conductive body B of the electrode support member ES' until reaching a seventh electrode comprising an arcuate region 250D of exposed metal within a second cutout C2D of the electrode support member ES′. An eighth electrode comprising a third wire conductor 300C having a distal end region of exposed wire 302D, wherein a lateral face of the exposed wire region 302D comprises the eighth electrode. The proximal end of the third wire conductor 300C is in operative electrical connection with a ground or low or negative terminal of the voltage generator. As current flows from the seventh electrode to the eighth electrode within the second cutout C2D of the electrode support member ES′, an electrical arc is generated across the spark gap and a pressure wave is produced. A portion of the third wire conductor 300C may be received within slot or channel 260 of ES with a proximal end of the third wire conductor 300C placed in operative electrical connection with the voltage generator as discussed above.

Similar configurations may be produced with three or more electrode support members connected in series. For example, a proximal electrode support member ES may be serially connected via a tantalum bridge wire to a second more distal electrode support member ES which may, in turn, be connected via a second tantalum bridge wire to a distal most electrode support member ES′. The current will flow through the first proximal electrode support member ES as described above, then through the second more distal electrode support member ES in the same way, then through the distal electrode support member ES' as described above.

It is also possible to combine two (or more) pairs of electrode support members connected in series, each pair of electrode support members functioning as described above, through use of a controller and/or multiplexer that selectively applies voltage to one pair of electrode support members, then selectively applying voltage to a second pair of electrode support members.

Moreover, in each of the wiring configurations for the embodiments of electrode support members discussed above, the current flow may be reversed within a given circuit by changing the polarity for a series of voltage pulses. In other embodiments, the polarity may be reversed for each voltage pulse. The result of such a polarity change is to change which side of the spaced-apart electrode pair functions as an anode and which side functions as a cathode. Functionally, this may provide an advantage in terms of extending the number of resultant electrical arcs may be generated between the two spaced-apart electrodes, particularly if one of the spaced-apart electrodes wears or erodes more quickly than the other electrode in the spaced-apart pair of electrodes.

FIG. 22 illustrates a handle that may be in operative communication and connection with a console that may comprise a processor and an operatively connected memory such as described in connection with FIG. 1 for executing instructions and the voltage pulse generator 110 described above. In this embodiment, the handle comprises, or is in communication with, an EPROM which may be in operative communication with a processor and/or a memory. The EPROM may provide therapy parameters, which may be specific for a particular model of IVL device, e.g., a balloon of specified length and/or with a specified number of electrode support members and/or spark gaps. In addition, the processor and/or memory may store therapy parameters for comparison of the progressing therapy monitored by the processor, against the stored therapy parameters. The processor may also generate connection logs, monitor number (and maximum number of allowed) pulses and may generate therapy logs. FIG. 23 provides a general flow for data involving an EPROM with an IVL system of the present disclosure.

Having described certain key features of the exemplary IVL systems herein, we now turn to the functional results of those exemplary systems. A KNOWN IVL system operated in conformance with its instructions for use, and a TEST system conforming with the present disclosure were subjected to a comparative pressure output test.

The testing method and materials included comparison of TEST and KNOWN IVL devices, each including a catheter, a balloon, two radially spaced-apart pairs of electrodes within the balloon and a voltage pulse generator connected with the pairs of electrodes. The TEST balloon size included 2.5×12 mm and 4.0×20 mm. The KNOWN balloon sizes included

2.5×12 mm, 4.0×20 mm, 2.5×40 mm and 4.0×40 mm. Each tested IVL system comprised substantially similar spacing between longitudinally spaced and adjacent electrode pairs. The testing device included ONDA HNR-0500 (S/Ns 2149, Cal date 3 May 2023 and 2160, Cal date 5 May 2023) needle hydrophones without amplification. This hydrophone has an active diameter of 2.5 mm. The hydrophone was calibrated and traceable to Onda Corporation. The frequency response is flat from 0.5 MHz to 10 MHz within +/−6 dB with measurement uncertainty of 1.5 dB for frequencies 0.5-1 MHz and 1 dB for frequencies 1-10 MHz. The TEST and KNOWN devices were immersed in a water bath. A total of 1,440 voltage pulses were executed by the KNOWN system and the resulting pressure output measured, and a total of 13,320 voltage pulses were executed and the resulting pressure output measured for the TEST system using the testing method and materials.

The pressure output test method consisted of the following steps for each tested device:
1. Place the subject IVL device balloon in a water bath.
2. Position the hydrophone about 2.89 mm distal of the subject spark gap with an approximate 5 mm offset between the hydrophone and the catheter's longitudinal axis.
3. Generate a voltage pulse at a predetermined magnitude.
4. Move the catheter such that the hydrophone is positioned about 0.9375 mm proximally relative to the subject pair of electrodes' spark gap.
5. Repeat steps 1-4 until the hydrophone is positioned more than 2.89 mm proximal of the subject spark gap.
6. Repeat steps 1-5 at axial rotations of the catheter of 45, 90, 135 and 180 degrees.

### Comparative Testing and Select Features

### Pressure Output Variability

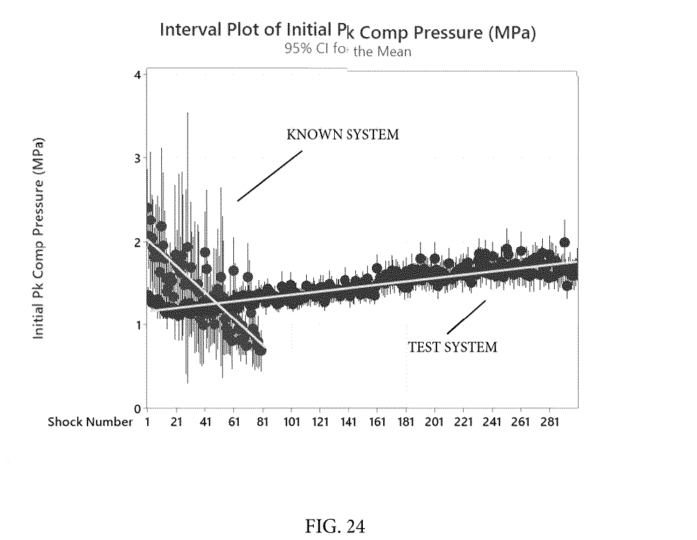

FIG. 24 provides a data plot of pressure output (initial peak pressures) for a KNOWN IVL system and a TEST IVL system that conforms with the present disclosure. As is immediately visually apparent, the KNOWN system's pressure output varies greatly. In contrast, the TEST system pressure output data is relatively tightly controlled, with relatively low variation compared with the KNOWN system's variation. A data summary is provided in Table 1:

TABLE 1

| | | | Standard | Coefficient of |
|---|---|---|---|---|
| | Number of | | Deviation | Variation (CV): |
| System | Tests | Mean (Mpa) | (SD) | SD/Mean |
| KNOWN | 1,440 | 1.2945 | 0.5475 | 39.3% |
| TEST | 13,320 | 1.4468 | 0.3441 | 23.8% |

Thus, the coefficient of variation (“CV”), which is an indicator of variability within data sets is 39.3% for the KNOWN system's pressure output data and is 23.8% for the TEST system's pressure output data. Accordingly, it can be reasonably concluded that the TEST system's pressure output data is more controlled and substantially less variable than the KNOWN system's pressure output data. Using the key features in an IVL system will therefore allow for a CV in a series of pressure output data that is less than 35%. More preferably, the CV in a series of pressure output data will be less than 30% and still more preferably, the CV in a series of pressure output data will be less than 25%. As noted supra, the IVL systems described herein provide for more durable (far more pulses/electrical arcs/pressure waves per catheter), more efficient (higher frequency of pulses/electrical arcs/pressure waves), and much more controlled pressure output from the produced pressure waves than the known IVL systems.

### Force Required to Traverse Vasculature

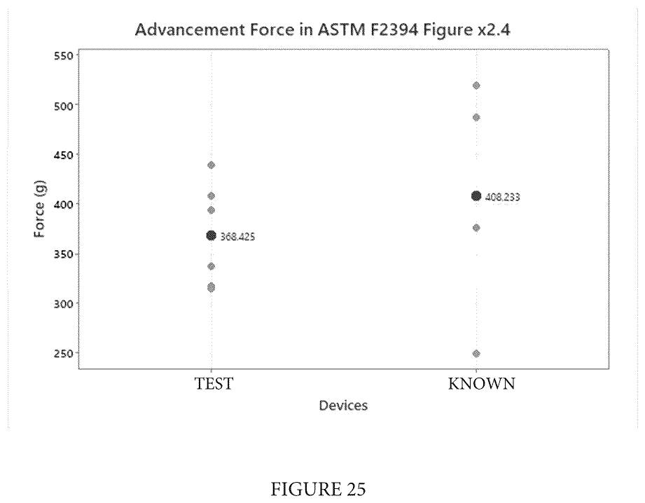

The TEST system and KNOWN system (Shockwave Medical C2 IVL catheter) were subjected to force standard tracking testing using the ASTM F2394 Tracking Fixture as shown in FIG. 25. The tested systems each comprises a total number of two emitters comprising spaced-apart electrodes. The tested systems were each translated through the illustrated standard fixture while tracking the force required to move the tested catheters from the beginning to the end of the fixture. The TEST and KNOWN systems were both two-emitter designs (two spaced-apart electrode pairs, wherein the spaced-apart electrode pairs are longitudinally spaced apart from each other), and the balloon sizes were both 3.0 mm×20 mm for both of the TEST and KNOWN systems. The ASTM F2394 Tracking Fixture was filled with water to lubricate. The TEST and KNOWN catheters were tracked over a wire through the model until the distal tip reached the end of the model. The peak force was the value that was recorded and observed in the plot as illustrated in FIG. 25. This comparative testing measures relative resistance through tortuous vasculature.

As shown, the mean peak force for the TEST system was 368.425 grams and for the KNOWN system, the mean peak force was 408.233 grams. This represents a 9.75% decrease in force required for the TEST system compared with the KNOWN system. The % difference between the two mean peak force values is 10.8%.

Accordingly, the mean peak force for embodiments of the TEST system through the ASTM F2394 Tracking Fixture is approximately 9.75% less than the mean peak force for the KNOWN system.

Certain embodiments of the TEST system may comprise a mean peak force through the ASTM F2394 Tracking Fixture that is at least 9.5% less than the mean peak force for the KNOWN system. Certain embodiments of the TEST system may comprise a mean peak force through the ASTM F2394 Tracking Fixture that is at least 9% less than the mean peak force for the KNOWN system.

Certain embodiments of the TEST system may comprise a mean peak force through the ASTM F2394 Tracking Fixture that is at least 8% less than the mean peak force for the KNOWN system.

Certain embodiments of the TEST system may comprise a mean peak force through the ASTM F2394 Tracking Fixture that is at least 6% less than the mean peak force for the KNOWN system.

Certain embodiments of the TEST system may comprise a mean peak force through the ASTM F2394 Tracking Fixture that is at least 5% less than the mean peak force for the KNOWN system.

Accordingly, the TEST system traversed the test fixture from a beginning to an ending of the fixture with a peak force less than 405 grams.

Further, the TEST system traversed the test fixture from a beginning to an ending of the fixture with a peak force less than 400 grams.

The TEST system also traversed the test fixture from a beginning to an ending of the fixture with a peak force less than 375 grams.

The TEST system also traversed the test fixture from a beginning to an ending of the fixture with a peak force less than 370 grams.

As a result, the peak advancement force for the TEST system may be within a range of about 400 grams to about 405 grams.

The peak advancement force for the TEST system may also be within a range of about 375 grams to about 405 grams.

The measured peak advancement force is for the TEST system may also be within a range of about 370 grams to about 405 grams.

In addition, Table 2 below provides a partial summary listing of functional improvements and enhancements provided by embodiments of the present disclosure relative to a KNOWN system, with a partial listing of the disclosed features leading to the improvements and enhancements.

TABLE 2

| | | | Presently Disclosed |
|---|---|---|---|
| | | Presently Disclosed | Features Enabling |
| Functional result | KNOWN system | Embodiments | the Function |
| Crossing profile | Outer diameter of | Outer diameter | 1. Electrodes not |
| | 0.044″-0.047″ | (“OSD”) about | fully circumferential; |
| | | 0.044″ or less for | wires in same |
| | | 2.5 mm diameter | circumferential |
| | | balloon. OD about | plane as the body of |
| | | 0.045″ or less for | the electrode |
| | | 3.0 mm and 3.5 mm | support member. |
| | | diameter balloons | 2. Tapering member |
| | | | in the proximal |
| | | | inflation region of |
| | | | the balloon. |
| Deliverability | | | 1. Smaller crossing |
| | | | profile in the folded |
| | | | balloon regions; less |
| | | | push force required. |
| | | | 2. Smaller distal |
| | | | outer diameter at |
| | | | distal tip than at the |
| | | | proximal end of the |
| | | | sealed balloon. |
| | | | 3. Increased |
| | | | flexibility in at least |
| | | | the balloon region |
| | | | compared with |
| | | | KNOWN-one last |
| | | | layer of material |
| | | | than known. |
| | | | 4. Improved axial |
| | | | push force without |
| | | | kinking due to |
| | | | presence of tapering |
| | | | outer member and |
| | | | stiffening layer in |
| | | | region of the |
| | | | balloon. |
| Efficiency/frequency | 1 pulse/second | 1 to 5 pulses/ | 1. Control of spark |
| of pulses | | second with 2 | gap lengths. |
| | | pulses/second | 2. Minimal spark gap |
| | | preferred | shorting. |
| | | | 3. Tantalum bridge |
| | | | wire. |
| | | | 4. Insulation |
| | | | covering electrode |
| | | | support bodies |
| | | | resulting in less gas |
| | | | produced during |
| | | | arcing. |
| Variability of | Greater than 39% | Less than 24% CV | 1. Control of spark |
| pressure output | CV | | gap lengths. |
| | | | 2. Minimal spark gap |
| | | | shorting. |
| | | | 3. Tantalum bridge |
| | | | wire. |
| | | | 4. Changing voltage |
| | | | to accommodate |
| | | | pressure over |
| | | | number of pulses. |
| Durability (maximum | Maximum 120 | Up to 500 pulses | 1. Control of spark |
| number of pulses per | pulses per catheter | per catheter for | gap lengths. |
| catheter). | for coronary | coronary and | 2. Minimal spark gap |
| | devices. Maximum | peripheral devices. | shorting. |
| | 300 pulses per | | 3. Tantalum bridge |
| | catheter for | | wire. |
| | peripheral devices. | | 4. Insulation |
| | | | covering electrode |
| | | | support bodies, |
| | | | resulting in less gas |
| | | | produced during |
| | | | arcing. |
| | | | 5. Less variable |
| | | | pressure output |
| | | | than known |
| | | | systems. |

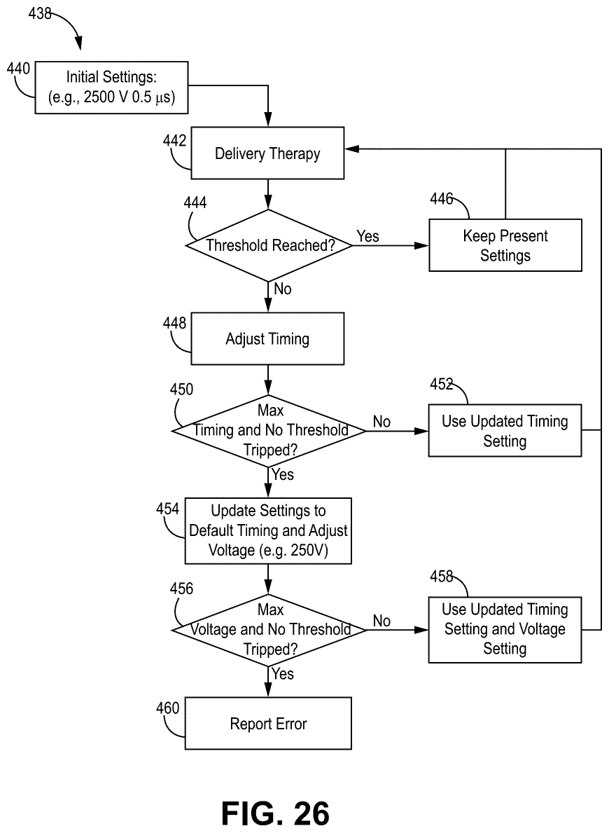

Referring now to FIG. 26, an exemplary flow diagram is shown concerning control in operation of an IVL system, specifically the number of voltage pulses generated and the magnitude of the generated pulses. Such control operations can be governed by the electric pulse generation system 20, and illustratively by the IVL control system 22 discussed above in connection with FIG. 1.

Referring now to FIG. 26, a flow diagram is shown concerning one embodiment of a control 438 in operation of IVL as discussed concerning boxes 440 through 460. Such control operations can be governed by the electric pulse generation system 20, and illustratively by the IVL control system 22. As discussed in additional detail herein, control 438 applies incremental changes in power parameters during cycling of applied voltage while monitoring parameters related to spark generation. For example, incrementally increasing the magnitude of the voltage that is applied to the discharge electrodes, and/or the duration of the voltage application, can increase the likelihood of generation of an effective spark without excessive energy.

Moreover, if increases to the duration of voltage applied to the discharge electrodes fails to generate sufficient spark, incremental increase to the voltage can further increase the likelihood of generation of an effective spark without excessive power. Still further, if incrementally increased voltage fails to generate sufficient spark, duration can once again be incrementally increased before further increasing voltage. Accordingly, it can be appreciated that controlled incremental increases in duration and voltage can be implemented to achieve effective spark generation, and thus pressure wave generation, at or near the lowest required power characteristics for effective spark generation. Increasing the likelihood of reaching sufficient spark with lower power can increase the efficiency, safety, and/or reduce intensity of effective IVL therapy.

In box 440, initial settings comprising an initial target voltage magnitude and duration of application are applied. For illustrative example, default initial settings are applied as a discharge voltage of 2500 volts (V) for a duration of 0.5 microseconds. In some embodiments, the initial settings may be determined by any suitable manner, including by use of programmable defaults, as adjusted settings based on use, for example, adjusted based on the patient characteristics, environmental conditions, procedural approach, and/or product cycle lifetime (i.e., operable age), among other aspects. From box 440, control may proceed to box 442.

In box 442, electrical energy is applied to the spaced-apart electrodes to deliver IVL therapy. In the first instance of proceeding from box 440 to box 442, electrical energy is applied at the initial settings, e.g., an applied voltage of 2500 V for a duration of 0.5 microseconds. When applied in a clinical setting, such that the IVL catheter is arranged within the patient's body lumen, and specifically with discharge electrodes submerged within a fluid medium within a fluid-filled member such as an angioplasty balloon, pressure wave therapy can ensue. However, as mentioned below, at present conditions, in some instances the energy provided at the initial settings may be insufficient to generate a spark at the electrodes, or may generate insufficient spark or insufficient pressure wave. From box 442, control may proceed to box 444.

In box 444, determination of threshold aspects is conducted. In one embodiment, a determined value for current applied in box 442 is compared with a predetermined threshold current value to determine and ensure that an electrical arc was formed between the spaced-apart electrodes. The predetermined threshold current value may be embodied as a predetermined fixed value, e.g., 20 amperes (amps), but in some embodiments, may have any suitable value, for example 50 amps, 100 amps, 150 amps, 175 amps, the threshold current value fora given cycle may be determined based on the number of previous cycles in a therapy session, the number of cycles maintaining present settings (e.g., through box 446) in a therapy session, the number of particular successive cycles (e.g., number of successive cycles through box 446 or box 448 or box 452 or box 454 or box 458), the patient characteristics, environmental conditions, procedural approach, and/or product cycle lifetime (i.e., operable age), among other aspects. Additionally, although the threshold current value may be set to one value, the actual current passed during a sufficient generation of arc across the electrodes may be greater. The mechanism for monitoring the current applied is discussed further below.

Responsive to determination that the determined value for current applied in box 442 is equal to or greater than the threshold current value, control may proceed to box 446. Otherwise, responsive to determination that the determined value for current applied in box 442 less than the threshold current value, control may proceed to box 448. For example, as mentioned above, insufficient spark generation may result in little or no current flow (e.g., about 0 to about 5 amps, which may represent dissipated energy into the medium without arcing) across the electrodes, which would not achieve the threshold current value, and thus would proceed to box 448.

In the illustrative embodiment, the determined current value for current applied in box 442 is embodied as the instantaneous current applied across the electrodes and may be determined for each applied voltage pulse. In some embodiments, the determined current value may be embodied as an aggregated value, such as a time-averaged value of current applied to the electrodes. Continuing from the exemplary embodiment applying a threshold current value of 20 amps, when 20 amps is achieved by the therapy delivered, the electrical arc is determined to have occurred and the initial settings of voltage magnitude and duration are sufficient to produce the desired electrical arc.

It can be appreciated that the substantial occurrence of 20 amps provides considerable current across the discharge electrodes indicating the generation of meaningful spark. By comparison little or no current may flow across the discharge electrode when a spark fails to generate or when insufficient spark is generated under the applied duration and at the applicable voltage, 2500 V by example, and which can generally range between about 500 V to about 5000 V for IVL therapy, although conceivably can range between about 100 V to about 10000 V in terms of practical application.

In box 446, determination is made to maintain presently selected settings. In the illustrative embodiment, the presently selected settings include the discharge voltage of and applicable duration as applied immediately previously in box 442. For avoidance of doubt, if the initial settings have just immediately been applied in box 442, resulting in 20 amps of current, the initial settings would be applied to the next voltage pulse and/or cycle or series of voltage pulses. However, if the presently selected settings include updated settings, for example, updated duration and/or voltage settings from later portions of control 438, as discussed in additional detail herein, then maintaining presently selected settings would include the immediately applied updated settings. Maintaining presently selected settings proceeds openly to return to box 442, to again apply electrical energy to the electrodes to deliver IVL therapy.

In box 448, if the current threshold is not met then a determination is made to increase the duration of applied voltage to the discharge electrodes. Continuing the example in which less than 20 amps of current indicates insufficient spark generation, rather than immediately increasing the voltage applied, the duration of applied voltage can be incrementally increased. At the microsecond-scale, increasing the duration of applied voltage can increase the likelihood of sufficient spark generation using the same voltage previously applied. This can be achieved as the result of overcoming threshold system impendence and/or other factors affecting the ease of spark generation during a given cycle at a given voltage.

The duration of applied voltage may be increased by a predetermined duration interval, illustratively embodied as a fixed value, e.g., 0.5 microseconds or other duration increase. In some embodiments, the predetermined interval for a given voltage pulse and/or cycle or series of voltage pulses may be determined based on factors such as the number of times therapy cycles have ensued previously during the therapy session (e.g., number of voltage pulses, cycles or series intervals have proceeding through boxes 442, 444, and 446, prior to proceeding to box 448), the patient characteristics, environmental conditions, procedural approach, and/or product cycle lifetime (i.e., operable age), among other aspects. In some embodiments, the predetermined duration interval for a given cycle may be varied by predetermined rate of change, for example, by percentage gain or loss by cycle. Control 438 then proceeds openly from box 448 to box 450.

In box 450, determination is made whether a maximum duration has been achieved. In some embodiments, the maximum duration is a predetermined duration embodied as a fixed value, e.g., 35 or more microseconds. By way of example, if the control sequence progresses through cycles from initial settings of box 440 at 0.5 microseconds through box 448, until reaching an exemplary maximum of 35 microseconds, the maximum duration would be achieved as a threshold value.

In some embodiments, the maximum duration for a given cycle may be determined based on the number of previous cycles in a therapy session, the number of cycles maintaining present settings (e.g., through box 446) in a therapy session, the number of particular successive cycles (e.g., number of successive cycles through box 446 or box 448 or box 452 or box 454 or box 458), the patient characteristics, environmental conditions, procedural approach, and/or product cycle lifetime (i.e., operable age), among other aspects. By deduction, in box 450, the threshold current value has not been achieved in the present cycle, although in some embodiments, affirmative determination and/or confirmation that threshold current value has not been achieved may be performed. Responsive to determination that the maximum duration has not been achieved, control proceeds to box 452. Otherwise, responsive to determination that maximum duration has been achieved, control proceeds to box 454.

In box 452, determination is made to apply the updated duration and return to delivery of therapy in box 442. In the illustrative embodiment, the duration has been updated in box 448 by increasing the presently selected duration by the predetermined duration interval, and determination to apply the updated duration confirms and proceeds with the updated duration. In the illustrative embodiment, the applied voltage remains as presently selected. Proceeding with the updated duration proceeds openly to return to box 442, to again apply electrical energy to the discharge electrodes to delivery therapy using the updated duration.

In box 454, the applied voltage is increased by a predetermined voltage interval. The presently selected setting for applied voltage is illustratively increased by the predetermined voltage interval. The presently selected duration is returned to the value at the initial setting, exampled as 0.5 microseconds, to be applied with the updated voltage, although in some embodiments, the updated duration may have any suitable value under newly updated voltage settings, for example, the updated duration may be determined based on the number of cycles of the therapy session when newly update voltages occur.

In the illustrative embodiment, the predetermined voltage interval is embodied as a fixed value of 250 V, such that, in the first exemplary occurrence of box 454, the presently selected applied voltage is increased to 2750 V from the value at the initial setting of 2500 V. In some embodiments, the predetermined voltage interval for a given cycle may be determined based on the number of previous cycles in a therapy session, the number of cycles maintaining present settings (e.g., through box 446) in a therapy session, the number of particular successive cycles (e.g., number of successive cycles through box 446 or box 448 or box 452 or box 454 or box 458), the patient characteristics, environmental conditions, procedural approach, and/or product cycle lifetime (i.e., operable age), among other aspects.

In box 456, determination is made whether maximum voltage has been achieved. In the illustrative embodiment, the maximum voltage is a predetermined voltage embodied as a fixed value, e.g., 3500 V. By way of example, if the control sequence progressed through cycles from initial settings of box 440 at 2500 V through box 454, until reaching 3500 V, the maximum voltage would be achieved as a threshold value.

In some embodiments, the maximum voltage for a given cycle may be determined based on the number of previous cycles in a therapy session, the number of cycles maintaining present settings (e.g., through box 446) in a therapy session, the number of particular successive cycles (e.g., number of successive cycles through box 446 or box 448 or box 452 or box 454 or box 458), the patient characteristics, environmental conditions, procedural approach, and/or product cycle lifetime (i.e., operable age), among other aspects. By deduction, in box 456, the threshold current value has not been achieved in the present cycle, although in some embodiments, affirmative determination and/or confirmation that threshold current value has not been achieved may be performed. Responsive to determination that maximum voltage has not been achieved, control proceeds to box 458. Otherwise, responsive to determination that maximum voltage has been achieved, control proceeds to box 460.

In box 458, determination is made to apply updated voltage. In the illustrative embodiment, the voltage has been updated in box 454 by increasing the presently selected voltage by the predetermined voltage interval, and determination to apply the updated voltage confirms and proceeds with the updated voltage. The duration was updated in box 445 to return to the value at the initial setting, exampled as 0.5 microseconds, and proceeds to be applied with the updated applied voltage. Proceeding with the updated voltage proceeds openly to return to box 442, to again apply electrical power to the electrodes to delivery therapy using the updated voltage and updated duration.

In box 460, determination of error occurs. Responsive to determination of error, an error message is provided. Such an error message illustratively terminates the therapy session, but in some embodiments, may take other safety and/or communication actions, for example, such as displaying an error communication to a user for consideration in determining remedial action.

In the illustrative embodiment, by deduction, responsive to error determination, the maximum voltage and maximum duration can be determined not to have generated a spark, although in some embodiments, insufficient spark generation may be determined. In some embodiments, responsive to error determination, failed and/or insufficient spark generation under maximum voltage and maximum duration may be determined by affirmative determination and/or confirmation. Following box 460, process control automatically terminate.

Within the discussion of the control 438, exemplary increases in duration have been mentioned, although in some instances, for example, in certain cycles of control 438, voltage level may be decreased, for example, in certain cycles of control 438. For example, duration and/or voltage may be changed in a given cycle according to control 438 to decrease incrementally to achieve appropriate conditions, for example, to achieve appropriate discharged energy as discussed in additional detail herein relative to consideration of the voltage levels of an energy storage system, for example, a capacitance system before and after discharge.

As discussed further herein, control system 22 may control the number of generated voltage pulses in a series (or a plurality of series) of voltage pulses. An acceptable voltage window comprises a predetermined starting voltage magnitude and a predetermined upper voltage magnitude. IVL control system further comprises a predetermined voltage magnitude for incremental increasing of voltage magnitude after each series of voltage pulses if the magnitudes of the executed voltage pulses is within the acceptable voltage window. Separate sets of predetermined control data may be provided in control system 22 for IVL systems comprising balloons that are of identifiable characteristics such as, without limitation, different outer diameter sizing, e.g., 2.5 mm, 3.0 mm, 3.5 mm and/or 4.0 mm.

An exemplary embodiment may comprise a 2.5 mm or a 3.0 mm balloon, wherein the control system 22 comprises control data including an exemplary starting target voltage of about 3000V (predetermined lower voltage threshold), voltage pulse series comprising 10 pulses, and an incremental voltage increase of 25V if the voltage pulse series magnitudes are less than an exemplary upper voltage threshold of about 3500V. As the skilled artisan will recognize, the incremental voltage increase may comprise any voltage magnitude, including without limitation within about 1V to about 250V. A voltage increase may comprise about 25V, but may be greater or less than 25V in certain embodiments. As the artisan will recognize, the predetermined starting voltage may be less than 3000V and the predetermined upper voltage threshold may be greater than 3500V. Thus, an exemplary starting voltage may, without limitation, comprise about 2500V and an exemplary upper voltage threshold may comprise about 4100V. In other embodiments, an exemplary predetermined starting voltage maybe greater than 3000V and an exemplary upper voltage threshold may be greater than about 3250V.

Another exemplary embodiment may comprise a 3.5 mm or a 4.0 mm balloon, wherein the control system 22 comprises control data including an exemplary starting target voltage of 2850V (predetermined lower voltage threshold), voltage pulse series comprising 10 pulses, and an incremental voltage increase of 25V if the voltage pulse series magnitudes are less than an exemplary predetermined upper voltage threshold of 3700V.

Another exemplary embodiment may comprise a 2.5 mm, 3.0 mm, 3.5 mm or 4.0 mm wherein the control system 22 comprises control data including an exemplary predetermined starting target voltage of about 2850V and an exemplary upper voltage threshold of about 3250V.

In some embodiments, the control system 22 may comprise an exemplary predetermined starting voltage magnitude and an exemplary upper voltage threshold that are within the range of about 2700V to about 3700V.

In some embodiments, the current produced by a voltage pulse may be within the range of 120 amps to 220 amps.

In some embodiments, a series of voltage pulses may be generated for delivery to one or more sets of spaced-apart electrodes 18, followed by a pause in generation of voltage delivery. Certain embodiments may comprise a pause between a last pulse in a series of voltage pulses and a first pulse in an immediately subsequent series of voltage pulses that is within the range of 5-20 seconds. More preferably, the pause between adjacent voltage pulse series may be about 10 seconds.

Some embodiments may comprise a predetermined time gap or duration between adjacent voltage pulses within a series of voltage pulses. In some embodiments, the time gap or duration between adjacent voltage pulses within a series of voltage pulses may be within the range of about 0.25 seconds (correspondent to a voltage pulse frequency of about 4 Hz) to about 1 second (correspondent to a voltage pulse frequency of about 1 Hz).

In some embodiments, a time gap or duration between adjacent voltage pulses within a series of voltage pulses may be about 0.5 seconds (corresponded to about 2 Hz).

In some embodiments, the pause of predetermined duration between adjacent series of voltage pulses may be greater in length or duration than the time gap or duration between adjacent voltage pulses within a series of voltage pulses.

In some embodiments, the width, or duration, of an applied voltage pulse may be within a range of about 20 to about 30 microseconds. In some embodiments, the width, or duration, of an applied voltage pulse may be about 25 microseconds.

In some embodiments, the predetermined number of voltage pulses within a series of voltage pulses may be within the range of 5 voltage pulses to 20, or more, voltage pulses. In some embodiments, the number of voltage pulses within a series of voltage pulses may be about 10 voltage pulses.

In some embodiments, a predetermined maximum number of voltage pulses that are allowed for any one of the catheter assemblies 14 may be within the range of 50 voltage pulses to 300 or more voltage pulses.

Each of the control data comprising at least the predetermined starting voltage, the upper voltage threshold, the width or duration of the generated and/or applied voltage pulse, the predetermined pause between adjacent voltage pulse series, the time gap between adjacent voltage pulses within a voltage pulse series, the number of generated voltage pulses required before increasing voltage, the magnitude of an increase in voltage, and/or the maximum number of voltage pulses allowed for a given catheter assembly 14 may be stored in, and executed by, the control system 22.

In other embodiments, as will be discussed below, an EPROM device may be connected to, or associated with, a handle that is in operative association with the catheter assembly. In these embodiments, the control data comprising at least the predetermined starting voltage, the upper voltage threshold, the predetermined pause between adjacent voltage pulse series, the time gap between adjacent voltage pulses within a voltage pulse series, and/or the maximum number of voltage pulses allowed for a given catheter assembly 14 may be stored in the EPROM which is in operative association with the control system 22. The EPROM may allow identification of a particular catheter assembly 14 which may be used with its own individualized set of control data, thus allowing highly flexible operation.

The control system 22 in the various embodiments may monitor data corresponding with the control data and, if the monitored data is not compliant with a stored range of values, or a within a threshold value, the control system 22 may notify the operator and may, in some embodiments, allow no further generation of voltage pulses.

With continued reference to FIGS. 1, 26, 27A and 27B, portions of the illustrative electric pulse generation system 20, including portions of the IVL control system 22, are disclosed herein including various features and/or circuitry which may be implemented as portions of circuitry 28, although in some embodiments, such systems 20, 22 may share components and/or have isolated components as applicable, for example, such that circuitry 28 is intended to be diagrammatic and may also represent circuitry embodied by system 20 alone as applicable. In the illustrative embodiment, the IVL control system 22 includes an adjustable energy storage system, illustratively an exemplary capacitance system, 512 for selective adjustment of the energy storage capacity or magnitude applied for providing electrical energy to the electrodes. We refer hereinafter to energy storage system 512, illustrated by but certainly not limited to, the capacitance system.

The energy storage system 512 receives charge electrical energy from a power source of the electric pulse generation system 20. The energy storage system 512 provides discharge electrical energy to the electrodes (e.g., illustratively via VCAP2 and grounding), as discussed in additional detail herein.

The adjustable energy storage system 512 illustratively includes a number, e.g., one or more, of energy storage units, exemplified as individual capacitors 514, defining an energy storage network. In the illustrative embodiment, each energy storage unit 514 may be sized to have the same energy storage capacity and may be arranged for parallel connection with the other energy storage unit(s) in the energy storage network, although in some embodiments, any suitable size and/or arrangement of the energy storage unit(s) may be provided to support variable energy storage for IVL therapy. A relay system 516 may be arranged in connection with at least some of the energy storage elements 514 of the network. The relay system 516 comprises one or more relays for selectively connecting energy storage element(s) 514 together to receive charge and to discharge electrical energy to the electrodes.

In the illustrative embodiment, the relay system 516 includes an engaged arrangement in which all energy storage elements 514 of the network are connected for IVL use. Energy storage element(s) 514, when connected for IVL use, may be connected with other portions of the electric pulse generation system 20 to exchange electrical energy under other control operations. For example, under typical charge control operations, energy storage elements 514 connected for IVL use by relay system 516 may receive charge from power supply, and/or under typical discharge control operation, energy storage elements 514 connected for IVL use by relay system 516 may provide discharge energy to the electrodes 18. Accordingly, it can be appreciated that the relay system 516 can selectively connect all energy storage element(s) 514 for use in the IVL therapy, to provide a maximum energy storage magnitude.

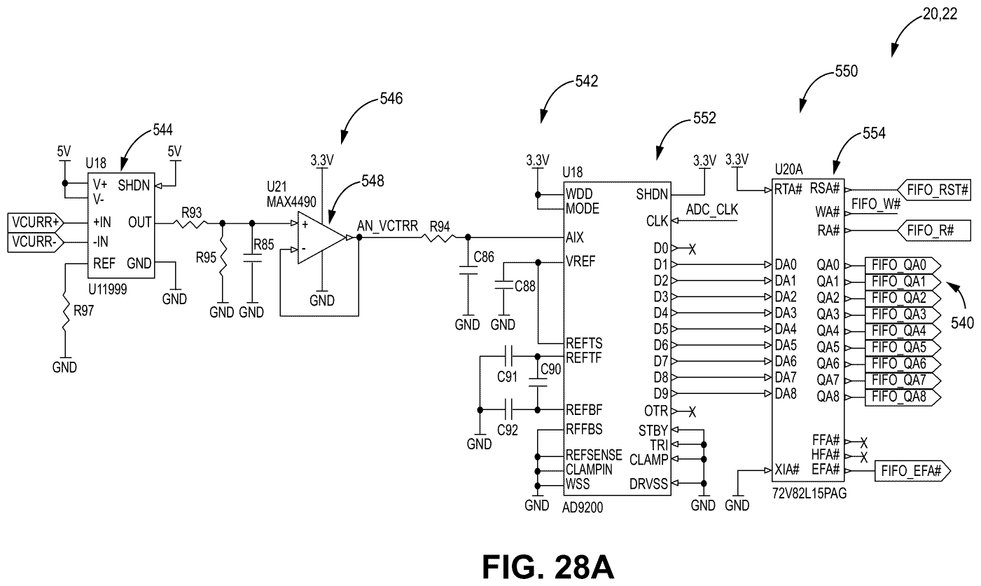

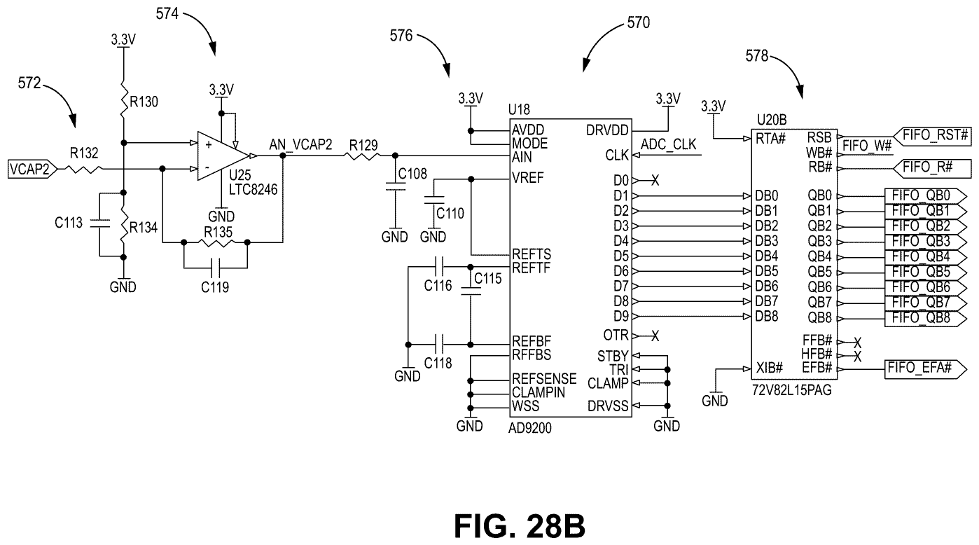

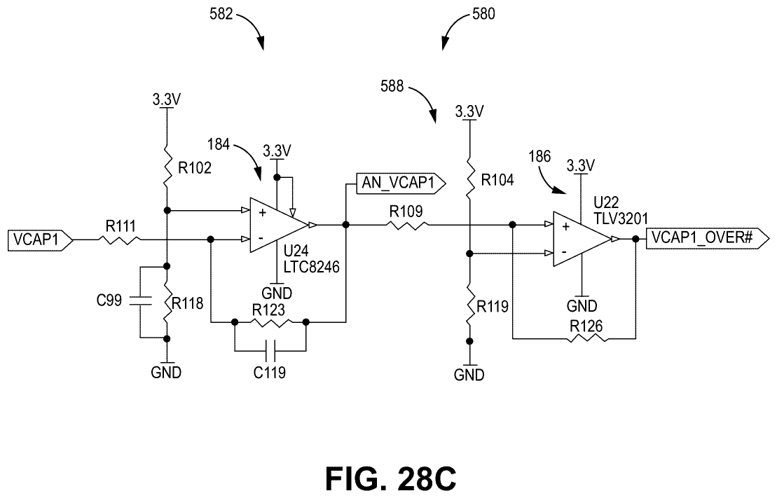

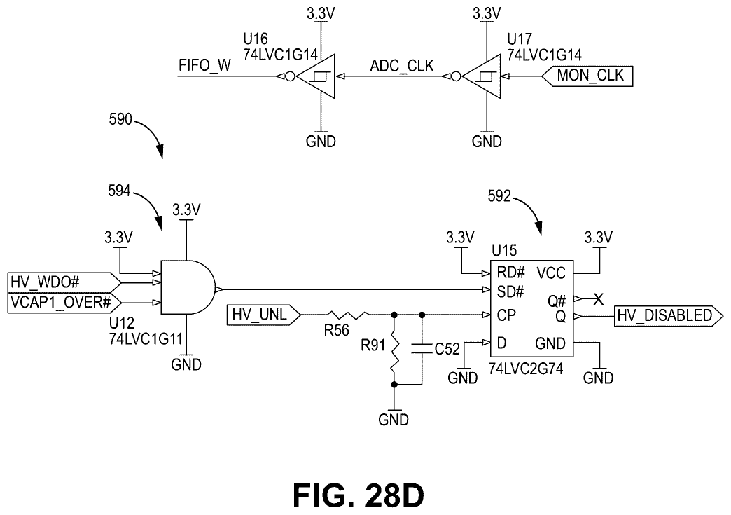

Additionally, the relay system 516 includes a disengaged arrangement in which fewer than all energy storage elements 514 of the network are connected for IVL use as suggested in FIGS. 28A-28D. A number of energy storage elements 514, illustratively two energy storage elements, are rendered disconnected from the other energy storage element(s) by disengagement of the relay system 516 for ease of description but without limitation. Energy storage elements 514 disconnected for IVL use by relay system 516 cannot receive charge from power supply, and/or under typical discharge control operation, energy storage elements 514 disconnected for IVL use by relay system 516 cannot provide discharge energy to the electrodes 18. Energy storage elements which are disconnected for use in IVL therapy may be discharged apart from the electrodes (e.g., for safe reduction of stored power), illustratively via diode 518 arranged in parallel with the relay system 516.

It can be appreciated that by adjustment of the energy storage capacity available to provide electrical energy to the electrodes, the total amount of energy provided to the electrodes can be controlled. The stored energy can be indicated according to ½C*V², where V represents the voltage and C represents the capacitance, such that applied electrical energy is directly proportional to the amount of energy connected in use. Accordingly, by selectively engaging the relay system 516 to close the circuit and connect the disconnected energy storage element(s), the energy storage capacity of the IVL system can be adjusted to provide variable energy levels to the electrodes. Although the illustrative embodiment includes relay control for connection and/or disconnection of a pair of energy storage elements, any suitable number of relays and/or energy storage elements may be applied to provide adjustable energy storage capacity for IVL therapy.

In the illustrative embodiment, the IVL control system 22 is configured to govern the applied stored energy. The IVL control system 22 illustratively determines the amount of stored energy to be applied, and upon determination that a change in the energy storage magnitude is desired, the IVL control system 22 operates the relay system 516 accordingly. For example, the IVL control system 22 may determine that one voltage pulse desires and/or requires lower energy storage magnitude and may communicate to operate the relay system 516 in the disengaged arrangement.

For one or more subsequent voltage pulses, the IVL control system 22 may determine that another voltage pulse desires and/or requires greater energy storage magnitude and may communicate to operate the relay system 516 in the engaged arrangement. For one or more further subsequent voltage pulses, the IVL control system 22 may determine that lower energy storage magnitude is again desired and/or required and may return the relay system 516 to the disengaged arrangement. Accordingly, the IVL control system 22 may operate the relay system 516 as needed to provide adjustable energy storage magnitudes for any given voltage pulse within a series of voltage pulses.

The applied energy storage may be adjusted on an ongoing basis, for example, for any given pulse. In practice, adjusting the energy storage capacity or magnitude may be conducted in conjunction with the level of voltage to be applied and/or in consideration of other aspects of power, efficiency, and/or technique. Moreover, under the lifetime of applied devices and systems, typical wear to components can alter the electrical and/or physical characteristics thereof, which may benefit from adjustment to the energy storage that is applied. For example, even small wear to electrodes can vary the spacing (gap) between a set of electrodes which can alter the conditions of the arc between electrodes. Accordingly, adjustable energy storage magnitude can accommodate variations in the electrodes and/or portions of the discharge system under repeated use, whether in individual therapy sessions or otherwise.

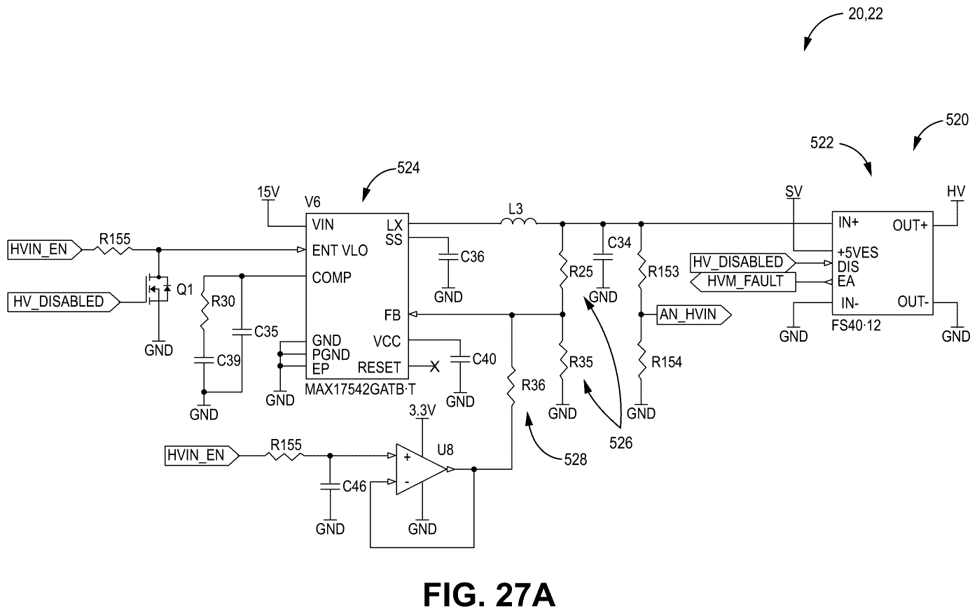

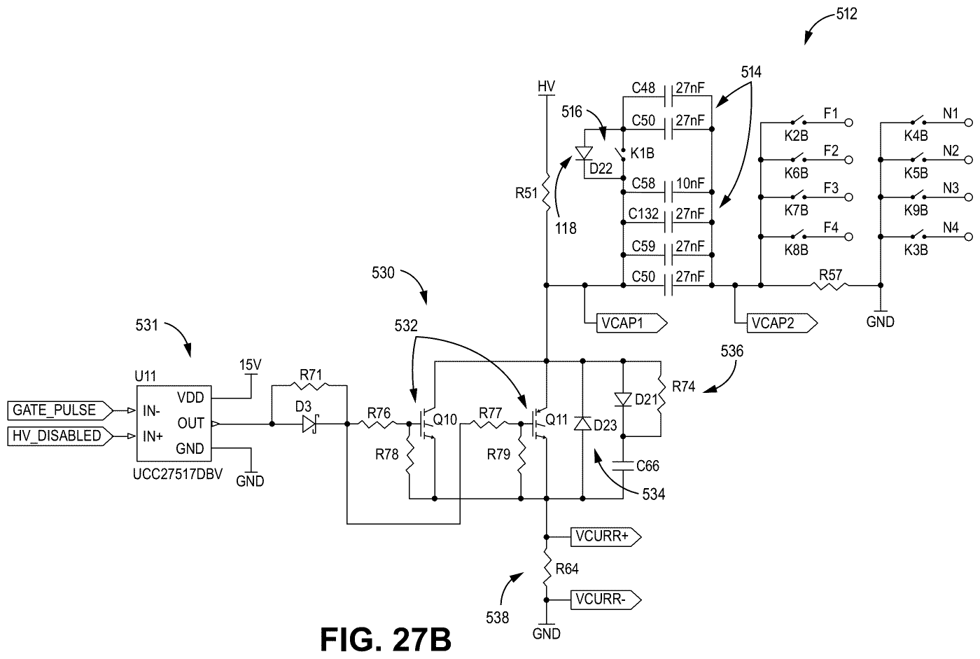

With continued reference to FIGS. 27A and 27B, embodiments of the disclosure are directed to systems and methods for adjusting the voltage provided to charge the energy storage system 512 as charge voltage. The charge voltage is illustratively provided as input to the energy storage system 512 as energy accumulation for discharge to generate a voltage pulse, controlled for variable charge. The charge voltage is illustratively controlled by a charge control system 520 of the IVL control system 22.

In the illustrative embodiment, the charge control system 520 provides accurate control of charge voltage via high frequency switched control signals from the processor 24. The switched control signal (“HVIN_VSET”), illustratively embodied as a pulse width modulated (PWM) signal, is amplified for control of high voltage supply. A high voltage DC/DC converter system 522 receives indication of the switch control signal within the range of about 0 V to about 12 V, and provides a corresponding charge voltage illustratively within the range of about 0 V to about 4000 V.

In the illustrated embodiment, the charge control system 520 includes a buck regulator system 524 for conditioning the low voltage power. The buck regulator system 524 is illustratively embodied as an integrated circuit (IC) to provide signal conditioning with low pass filtering and buffering of the PWM signal. The buck regulator system 524 receives a conditioned PWM signal with feedback for providing controlled low voltage power to the converter system 522 for applying high voltage power.

Resistors 526 can scale down the feedback voltage appropriately for the IC operation, to provide a variable feedback voltage to the buck regulator system 524 within a range of about 0 V to about 3.3 V at the additional resistor 528. As the duty-cycle of the PWM signal increases from zero to 100 percent, the filtered signal increases from zero volts to 3.3 volts, which can increase the current provided to the feedback network, and can require less voltage to achieve regulation, for example, at resistors, inductors, and/or capacitors arranged between the buck regulator system 524 and the converter system 522.

Referring still to FIGS. 27A and 27B, embodiments of the disclosure are directed to systems and methods for controlling the active time for discharge voltage pulses provided to the electrodes. A switching signal (e.g., “GATE_PULSE”) is provided by the processor 24 for high voltage switching via low voltage signaling. In the illustrative embodiment, the switching signal is applied to precisely activate a discharge switch system 530. The discharge switch system 530 is illustratively embodied to implement a driver 531 and semiconductor devices 532 as gate switches.

The gate switches 532 are illustratively embodied as insulated gate bipolar transistors (IGBT) having n-type gate-controlled arrangement. On active state of the switching signal to the driver 531, the gate switches 532 are activated into their conducting state to communicate discharge of energy from the energy storage system 512 to the electrodes. On inactive state of the switching signal the gate switches 532 are deactivated into their non-conducting states, blocking discharge of the energy storage system 512 to the electrodes.

In the illustrative embodiment, the gate switches 532 are arranged as active high devices, although in some embodiments, may be implemented as active low devices. Gate-controlled, active high IGBTs can provide precision control of discharge from the energy storage system 512, but may be implemented by any suitable manner including by other suitable semiconductors (e.g., p-type, FET, etc.) and/or other control designs (e.g., collector, emitter control, etc.).

In the illustrative embodiment, the discharge switch system 530 includes an anti-parallel diode 534 arranged to reduce reverse-voltage stresses on the gate switches 532. A disable signal (“HV_DISABLED”) is provided to the driver 531 which permits discharging of energy to the electrodes on inactive (low) signal, and may activate (high) to disable high voltage discharge under direction of the processor 24 and/or other safety systems. The snubber system 536 is illustratively embodied as a resistor-capacitor-diode (RCD) snubber network arranged to reduce voltage transients that may exceed rated voltages of various high-voltage components.

Referring now to FIGS. 28A-28D, the IVL control system 22 illustratively includes electrical power monitoring system 540. The electrical power monitoring system 540 is configured to conduct monitoring of various parameters of electrical power of the IVL device and systems, illustratively including sensing of current and voltage delivered to the electrodes, and voltage of the adjustable energy storage system 512.

The electrical power monitoring system 540 illustratively includes a current monitoring system 542. The current monitoring system 542 is embodied to sense the current delivered to the electrodes for a given voltage pulse. As discussed in additional detail herein, the current delivered to the electrodes can be considered in determining the power characteristics for subsequent voltage pulses.

In the illustrative embodiment as shown in FIGS. 28A-28D, the current monitoring system 542 receives indication of the voltage levels applied in each voltage pulse for determining current delivered to the electrodes. Returning briefly to FIGS. 27A and 27B, a shunt resistor 538 is arranged within the high-voltage current path establishing a proportional voltage (e.g., “VCURR+”, “VCURR−”). The proportional voltage is communicated to the current monitoring system 542 as shown in FIGS. 28A-28D.

A chip comprising an amplifier 544 is arranged to scale-up the proportional voltage, and provides the analog result to a conditioning network 546, embodied to include a resistor-capacitor network for scaling and/or filtering. The conditioning signal is buffered by a buffering amplifier 548, the output of which is provided to an analog-to-digital conversion (ADC) system 550 for digital conversion.

The ADC system 550 illustratively includes a converter 552 and memory 554. In the illustrative embodiment, the converter 552 provides digital output from the analog input, and the memory 554 is embodied as a first-in-first-out (FIFO) device for intermediate storage of digital outputs. The memory 554 illustratively receives the same clock signal driving the converter 552, to allow quick sampling of a number of measurement points with low-jitter. Memory outputs are provided to the processor 24 for consideration in overall IVL therapy control.

The IVL control system 22 illustratively includes a current monitoring system which compares the output signal (“VCURR”) generated by the chip comprising an amplifier 544 with a threshold value. The threshold value is embodied to be generated by a variable duty cycle PWM signal (“ISNS_ISET”) from the processor 24. The PWM signal can be low-pass filtered and/or buffered before delivery to a comparator. Responsive to the measured current exceeding the threshold value, the current monitoring system can assert an error signal (e.g., “ISNS OVER #”) to avoid overcurrent conditions.

The electrical power monitoring system 540 illustratively includes a voltage monitoring system 570. The voltage monitoring system 570 is embodied to sense the voltage between the set of electrodes. As discussed in additional detail herein, the voltage between the electrodes for a given pulse can be considered in determining the power characteristics for subsequent voltage pulses.

In the illustrative embodiment, the voltage monitoring system 570 includes a resistor network 572 arranged to attenuate the (switched) voltage of one of the electrodes of a set (e.g., “VCAP1”). The attenuated signal is provided to an op amp network 574 for filtering and offsetting for output to digital conversion. The output from the op amp network 574 is provided to the ADC conversion system 576 for digitization, including storage in FIFO memory 578 for access by the processor 24.

The electrical power monitoring system 540 may illustratively include an energy storage capacity voltage monitoring system 580 configured for monitoring the voltage within the adjustable energy storage system 512. Monitoring the voltage of the energy storage system 512 can allow determination of the stored energy of the energy storage system 512. Moreover, comparison of the stored energy of the energy storage system 512 before and after discharge can provide indication of the total energy delivered during a given discharge cycle. Such total energy data can be considered to increase confidence in determining whether a sufficient spark has been generated for IVL therapy.

In the illustrative embodiment, the voltage monitoring system 580 includes a voltage limiting system configured to monitor net voltage of the energy storage system 512 during charging. In the illustrative embodiment, voltage monitoring is discussed relative to the connected energy storage elements 514, more specifically, those energy storage elements which are connected to provide controlled discharge energy for IVL therapy, and not energy storage elements which are disconnected via the relay system 516 if any.

The voltage monitoring system 580 receives indication of the voltage of the energy storage system 512 during charging (“VCAP1”). The system 580 illustratively includes an amplifier configuration 582 comprising an amplifier 584 and a comparator 586. The comparator 586 is illustratively arranged to compare to the voltage with a fixed voltage, and to responsively trigger a signal (e.g., “VCAP1_OVER #”) when the voltage of the energy storage system 512 exceeds the fixed voltage. In the illustrative embodiment, the fixed voltage is embodied as setpoint generated by a resistor-resistor-capacitor network 588 above normal operation, but before damage will occur to various HV components.

An intermediate voltage of this circuit (e.g., “AN_VCAP1”) can be used to monitor progress during the charging cycle of the energy storage system 512. The intermediate voltage illustrative represents a heavily attenuated indication of the high voltage provided by the energy storage system to the electrodes (“VCAP_1”). Such attenuated signal can allow monitoring of high voltage systems while handling lower voltage indications thereof.

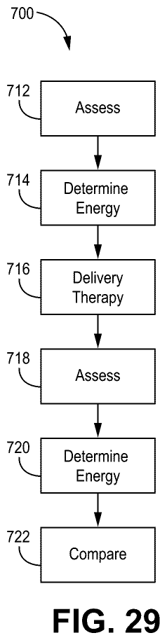

Referring now to FIG. 29, and with continued reference to FIGS. 1 and 26, the IVL system 12 can consider energy of the energy storage system 512 in operation. By monitoring energy of the energy storage system 512 before and after a discharge event, indication of the generation (and/or sufficiency) of spark can be determined as discussed in additional detail regarding the illustrative embodiment with reference to operation 700 concerning boxes 712 through 722.

In box 712, assessment of the energy storage system 512 is conducted. In the illustrative embodiment, the assessment includes determination of a voltage of the energy stored by the energy storage system 512. As mentioned above, the voltage monitoring system 580 can monitoring voltage of the energy storage system 512, for example, via the voltage limiting system during charging. In some embodiments, the assessment may include determination of any other suitable parameters to support energy monitoring of the energy storage system 512.

In box 714, the energy of the energy storage system 512 is determined. In the illustrative embodiment, the energy of the energy storage system 512 is determined based on the measured voltage according to

$$\frac{1}{2} C * {V}^{2} .$$

Accordingly, the processor 24 can compute the current energy of the energy storage system 512, including the energy stored just before discharge of energy to the electrodes.

In box 716, IVL therapy can be attempted. In the illustrative embodiment, a voltage pulse can be delivered to the electrodes. The voltage pulse can be applied according to the control arrangement as mentioned herein, for example, based on a determined duration in a control sequence.

In box 718, assessment of the energy storage system 512 is conducted. Assessment of the energy storage systems 512 in box 718 is embodied as occurring immediately after attempted IVL therapy in box 716 to provide an indication of the energy state of the energy storage system 512 immediately after (attempted) discharge to the electrodes. In the illustrative embodiment, the assessment includes determination of a voltage of the energy stored by the energy storage system 512, embodied as conducted by the voltage monitoring as mentioned above, although in some embodiments, assessment of the energy storage system 512 in box 718 may differ from box 712 in methodology and/or practice.

In box 720, the energy of the energy storage system 512 is determined. In the illustrative embodiment, the energy of the energy storage system 512 is again determined based on the measured voltage according to

$$\frac{1}{2} C * {V}^{2}$$

just as in box 714, yet after attempted delivery of IVL therapy. In some embodiments, determining energy of the energy storage system 512 in box 720 may differ in methodology and/or practice from that in box 714. Accordingly, the processor 24 can compute the current energy of the energy storage system 512, including immediately after (attempted) discharge of energy to the electrodes.

In box 722, comparison between energy determinations is conducted. The amount of energy determined within the energy storage system 512 in box 714 is illustratively subtracted from the amount of energy determined within the energy storage system 512 in box 720, such that the result represents the amount of energy discharged from the energy storage system 512 under a single attempt to deliver IVL therapy.

The amount of energy discharged can be considered to determine whether a spark (or sufficient spark) has occurred such that IVL therapy occurs. In the illustrative embodiment, a threshold energy discharge represents a discharged energy level which confidently indicates spark sufficient for IVL has occurred. Accordingly, in box 722, comparing the stored energy levels before and after discharge to determine whether the threshold energy discharge has been achieved can indicate spark for IVL therapy.

In the illustrative embodiment, and with reference to FIG. 26, the threshold energy discharge is a fixed predetermined value, for example, 600 millijoules (e.g., 3700 V, 90 nanofarad). However, in some embodiments, the threshold energy level for a given cycle may be determined based on the number of previous cycles in a therapy session, the number of cycles maintaining present settings (e.g., through box 446 of FIG. 26) in a therapy session, the number of particular successive cycles (e.g., number of successive cycles through box 446 or box 448 or box 452 or box 454 or box 458), the patient characteristics, environmental conditions (e.g., location within patient's body such as above the knee or above the knee), procedural approach, and/or product cycle lifetime (i.e., operable age), among other aspects.

Comparison of energy levels of the energy storage system 512 which indicates a spark for IVL therapy has occurred can responsively cause further therapy at the same duration, energy level, threshold characteristic, and/or threshold adjusted for other parameters. Comparison of energy levels of the energy storage system 512 which indicates a spark for IVL therapy has not occurred can cause adjustment to the duration and/or energy level applied, for example, as discussed concerning control operation 438.

In some embodiments, the threshold current value can be applied together with the threshold energy discharge, such that either threshold can individually indicate a spark for IVL therapy. In some embodiments, both thresholds may be required to be met to indicate a spark for IVL therapy.

Consideration of the energy states of the energy storage system 512 can provide desirable monitoring of IVL operations. For example, such monitoring can be less intrusive by reducing the need for direct measurements at the electrodes. Moreover, in high power applications, reliable consideration of the energy states can promote confidence over mere direct measurement in unpredictable high energy arc scenarios.

The IVL control system 22 illustratively includes an external watchdog system configured to assist in safe operation. The watchdog system includes an integrated circuit configured to trigger an error under lack of a timely toggled input signal to ensure appropriate high voltage operation. In some embodiments, the watchdog system may be formed externally including processor, memory, and/or circuitry distinct from or shared with the IVL control system 22.

Returning to FIGS. 27A and 27B, in the illustrative embodiment, the IVL control system 22 includes an umbrella monitoring system 590 configured to assist in safe operation. The umbrella monitoring system 590 illustratively includes a flip flop 592 and logic gate 594 for consideration of monitoring signals. The logic gate 594 is arranged to receive monitoring signals, embodied as energy storage system overvoltage (“VCAP1_OVER #”) from the voltage monitoring system 580, high voltage warning (“HV_WDO #) from the watchdog system, and in some embodiments, may receive overcurrent from the current monitoring system (“ISNS_OVER #”).

The logic gate 594 is embodied as an AND gate and the flip flop 592 embodied as an asynchronous D-flip flop, such that activation signals from the gate 594 which last longer than the minimum clock pulse width of the flip flop 592 cause an assertion of outputs to disable high voltage output (e.g., “HV_DISABLED”), but activation signals from the gate 594 shorter than the minimum clock pulse width of the flip flop 592 do not raise disabling outputs from the umbrella monitoring system 590.

Assertion of the signal to disable high voltage output (“HV_DISABLED”) is illustratively provided to the discharge switch system 530 to disable voltage pulse switch activation to the electrodes. In the illustrative embodiment, the disabling output signal is provided to the driver 531 and indirectly to alter on/off operation of the gate switches 532. Such disabling output signal is illustratively provided to low-voltage supplies, e.g., buck regulator system 524, and high voltage modules, e.g., converter system 522.

Accordingly, the logic gate 594 receives monitoring signals discussed above comprising: (1) energy storage system overvoltage (“VCAP1_OVER #”) from the voltage monitoring system 580, (2) high voltage warning (“HV_WDO #) from the watchdog system, and in some embodiments, (3) may receive overcurrent from the current monitoring system (“ISNS_OVER #”). These monitoring signals ae also connected to a three-input AND logic gate [U12], which is upstream of a D flip-flop with asynchronous set and reset functionality [U15], so that any signal that asserts for longer than the minimum pulse width of the flip-flop [U15] will cause its outputs [HV_DISABLED, HV_DISABLED #] to assert. These signals travel downstream and inhibits the operation of the gate driver [U11], variable low-voltage supply [U6], high-voltage module [U7], and slightly changes the turn-on and turn-off operation of the switching devices [Q10, Q11] via transistors [Q12, Q13].” This system allows any one of monitoring signals to disable the output of the system if asserted longer than an established duration.

Within the present disclosure, the ability to run off of AC mains or battery DC can afford versatility of power and control for IVL therapy. Unlike known IVL systems, certain embodiments within the present disclosure can avoid sitting idle until fully (or substantially) recharged in order to be applied in IVL therapy, for example, if insufficiently charged by the time IVL therapy is desired. Accordingly, such costly delays, or interruptions, in procedure can be avoided with embodiments of the present disclosure. The electric pulse generation system 20 illustratively includes a battery power storage system, and is configured to selectively charge the energy storage system 512 from battery stored energy only, from the battery power storage system while connected to mains, such as outlet power, or directly from DC power converted from the AC mains without the battery power storage system. When plugged in to AC mains, regulated DC power is delivered directly to the high voltage systems. In operational states in which the current demand for IVL operations is high, the battery power storage system charge current can be reduced to allow for higher IVL operations system current. When not plugged in to AC mains, battery power can be delivered directly to energy storage system 512. In the illustrative embodiment, systems and devices of power management, including for example, inverters, conditioners, power storage devices, and/or related aspects may be comprised by the electric pulse generation system 20 to provide applicable power to the IVL control system 22.

Examples of suitable processors may include one or more microprocessors, integrated circuits, system-on-a-chips (SoC), among others. Examples of suitable memory, may include one or more primary storage and/or non-primary storage (e.g., secondary, tertiary, etc. storage); permanent, semi-permanent, and/or temporary storage; and/or memory storage devices including but not limited to hard drives (e.g., magnetic, solid state), optical discs (e.g., CD-ROM, DVD-ROM), RAM (e.g., DRAM, SRAM, DRDRAM), ROM (e.g., PROM, EPROM, EEPROM, Flash EEPROM), volatile, and/or non-volatile memory; among others. Communication circuitry 58 includes components for facilitating processor operations, for example, suitable components may include transmitters, receivers, modulators, demodulators, filters, modems, analog/digital (AD or DA) converters, diodes, switches, operational amplifiers, and/or integrated circuits. In some embodiments, memory 26 may represent one or more memory devices operable for IVL therapy operation. For example, each memory (e.g., 554, 578) may be included as part of memory 26, shared, or isolated therefrom.

Within the present disclosure, consideration of a set of discharge electrodes has been discussed in the context of a pair of electrodes, one of which may serve as a cathode and the other of which may serve as an anode, at a given instance. However, the number of electrodes in a set may be greater than a pair, for example, including one or more cathodes communicating with one or more anodes. Additionally, devices, systems, and methods within the present disclosure may include more than one communicating group of electrodes, whether electrically arranged serially, parallel, or independently from each other.

Power control operations disclosed herein may be applied equally, simultaneously, and/or sequentially to individual sets or groups of electrodes, in a given IVL therapy cycle. For example, threshold current value may be applied collectively to all deployed electrodes or to individual groups or sets of electrodes. Determinations made with respect to power control may be applied equally to related electrodes, or may be individualized to groups or sets of electrodes, in a given IVL therapy cycle. Within the present disclosure, supporting components, such as power supplies, sensors, and other implementing structures and/or features for performing IVL operations as disclosed herein are embodied as sub-portions of the electric pulse generation system 20 and/or IVL control system 22, for example, as parts of circuitry and/or instructions.

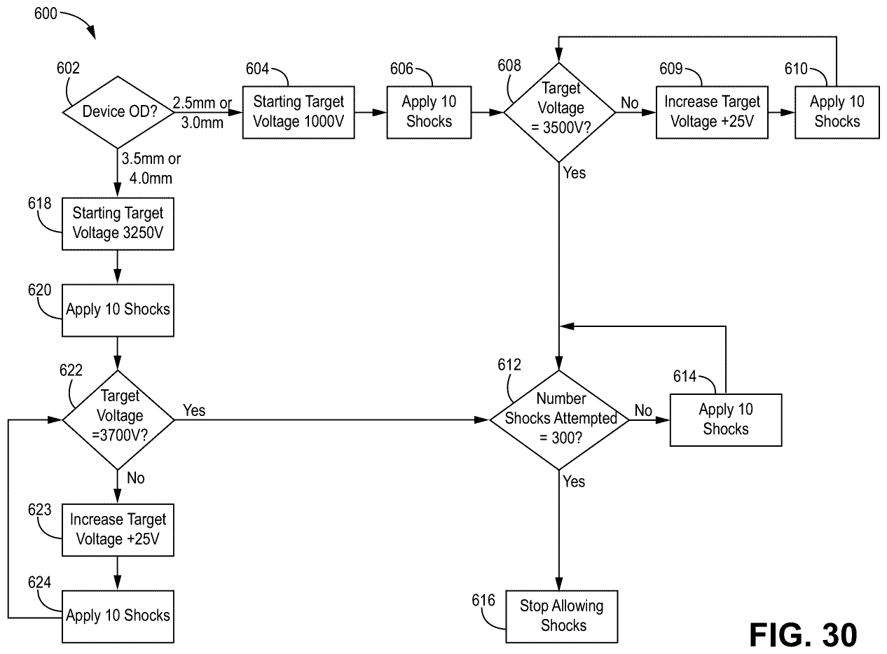

Referring now to FIG. 30, an exemplary flow diagram is shown concerning another embodiment of a control 200 in operation of an embodiment of an IVL system, specifically the number of voltage pulses generated and the magnitude of the generated pulses. It is understood that the control 200 may be combined with aspects of the control system and method embodiments described above in relation to FIGS. 1 and 26-29.

Control operations of control 200 may be governed by the electric pulse generation system 20, and illustratively by the IVL control system 22 discussed above in connection with FIG. 1. As discussed further herein, control system 22 may control the number of generated voltage pulses in a series (or a plurality of series) of voltage pulses. An acceptable voltage window comprises a predetermined starting voltage magnitude and a predetermined upper voltage magnitude. IVL control system further comprises a predetermined voltage magnitude for incremental increasing of voltage magnitude after each series of voltage pulses if the magnitudes of the executed voltage pulses is within the acceptable voltage window. Separate sets of predetermined control data may be provided in control system 22 for IVL systems comprising balloons that are of identifiable characteristics such as, without limitation, different sizing, e.g, 2.5 mm, 3.0 mm, 3.5 mm and/or 4.0 mm.

An exemplary embodiment of an IVL system may comprise a 2.5 mm or a 3.0 mm balloon, wherein the control system 22 comprises control data comprises an exemplary starting target voltage of 3000V (predetermined lower voltage threshold), voltage pulse series comprising an exemplary 10 pulses, and an exemplary incremental voltage increase of 25V if the voltage pulse series magnitudes are less than an exemplary upper voltage threshold of 3500V. As the skilled artisan will recognize, the incremental voltage increase may comprise any voltage magnitude, including without limitation within 1V to 250V. An exemplary voltage increase may comprise 25V, but may be greater or less than 25V in certain embodiments. As the artisan will recognize, the predetermined starting voltage may be less than 3000V and the predetermined upper voltage threshold may be greater than 3500V. Thus, an exemplary starting voltage may, without limitation, comprise 2500V and an exemplary upper voltage threshold may comprise 4100V. In other embodiments, an exemplary predetermined starting voltage may be greater than 3000V and an exemplary upper voltage threshold may be greater than 3250V.

Another exemplary embodiment may comprise a 3.5 mm or a 4.0 mm balloon, wherein the control system 22 comprises control data comprises an exemplary starting target voltage of 3250V (predetermined lower voltage threshold), voltage pulse series comprising 10 pulses, and an incremental voltage increase of 25V if the voltage pulse series magnitudes are less than an exemplary predetermined upper voltage threshold of 3700V.

With continued reference to FIG. 1, and in some embodiments FIG. 26, FIG. 30 illustrates initiation of a voltage pulse generation and control system 200 that begins with box 602 which requires determination of a particular balloon characteristic of interest, for example the outer diameter (“OD”) of the IVL system's balloon. In a first embodiment, if the balloon's outer diameter is, e.g., 2.5 mm or 3.5 mm, then in box 604 the starting voltage is set to 3000V, also referred to as the predetermined lower voltage threshold of the acceptable voltage magnitude window). The IVL therapy is initiated in box 606 by application of a series of voltage pulses (or shocks) from the electric pulse generation system 20 wherein each voltage pulse travels to the electrodes 18 within the balloon 16. If, in box 608, the target voltage magnitude is not at the predetermined upper voltage threshold, e.g., 3500V, then as in box 609, the target voltage is increased by an exemplary 25V (from 3000V to 3025V) and another series of voltage pulses (in this case 10 pulses) is executed, as in box 610 at 3025V. This process continues with cycling between boxes 608, 609 and 610 until the target voltage is at 3500V. When the target threshold voltage, or predetermined upper voltage threshold, is reached, and/or in some embodiments a predetermined maximum or desired number of voltage pulses, e.g., 300 pulses (or shocks) have been generated, then as in box 612, control system 22 determines if the number of generated voltage pulses (or shocks) in the plurality of series of voltage pulses has reached a maximum, or desired, number of pulses, e.g., 300 voltage pulses. If the maximum or desired, e.g., 300, voltage pulse threshold has not been reached, then as in box 614 another series of, e.g., 10, voltage pulses (or shocks) are applied. When the maximum or desired, e.g., 300 voltage pulse threshold has been reached, then as in box 616, additional voltage pulses (or shocks) are not allowed.

The predetermined maximum number of voltage pulses in various embodiments of the present disclosure may be within the range of 10-300 voltage pulses. The exemplary embodiments discussed herein comprise a predetermined maximum number of voltage pulses equal to 300 pulses. In other embodiments, the maximum number of voltage pulses may be greater than 300 pulses.

In a second embodiment, with continued reference to FIG. 1, and in some embodiments, FIG. 26, if the balloon's outer diameter is determined in box 602 to be, e.g., 3.5 mm or 4.0 mm, then the electric pulse generation system 20 initiates therapy at box 618 the starting voltage is set to 3250V, also referred to as the predetermined lower voltage threshold of the acceptable voltage magnitude window). The IVL therapy is initiated in box 620 by application of a series of voltage pulses from the electric pulse generation system 20 wherein each voltage pulse travels to the electrodes 18 within the balloon 16. If, in box 622, the target voltage magnitude is not at the predetermined upper voltage threshold, e.g., 3500V, then as in box 623, the target voltage is increased by an exemplary 25V (from 3250V to 3275V) and another series of voltage pulses (in this case 10 pulses) is executed, as in box 624, at 3275V. This process continues with cycling between boxes 622, 623 and 624 until the target voltage is at 3700V. When the target threshold voltage, or predetermined upper voltage threshold, is reached, and/or in some embodiments a maximum or desired number of pulses, e.g., 300 pulses (or shocks when applied to the one or more pairs of spaced-apart electrodes) have been generated, then as in box 612, control system 22 determines if the number of generated voltage pulses (or shocks) in the plurality of series of voltage pulses has reached a maximum, or desired, number of pulses, e.g., 300 voltage pulses. If the maximum or desired, e.g., 300, voltage pulse threshold has not been reached, then as in box 614 another series of, e.g., 10, voltage pulses (or shocks) are applied. When the maximum or desired, e.g., 300 voltage pulse threshold has been reached, then as in box 616, additional voltage pulses (or shocks) are not allowed.

Alternatively, the physician conducting the IVL therapy according to the voltage pulse generation and control system 600 may determine that the therapy is complete at a point during execution of the therapy. If the therapy is determined to be completed, then the physician may terminate the process of the voltage pulse generation and control system 600 at any point.

In some embodiments, the voltage pulse generation and control system 600 may comprise modification of the duration of applied voltage within, or across, one or more of the plurality of series of voltage pulses in accordance with the embodiments discussed above in connection with FIG. 26. For example, duration may be increased, or decreased, by a predetermined duration interval, illustratively embodied as a fixed value, e.g., 0.5 microseconds.

In some embodiments, the predetermined interval for given cycle may be determined based on factors such as the number of times therapy cycles have ensued previously during the therapy session (e.g., number of series of voltage pulses that have been executed by proceeding through boxes 606, 608 and 610, or 620, 622 and 624), the patient characteristics, environmental conditions, procedural approach, and/or product cycle lifetime (i.e., operable age), among other aspects. In some embodiments, the predetermined duration interval for a given cycle may be varied by predetermined rate of change, for example, by percentage gain or loss by cycle.

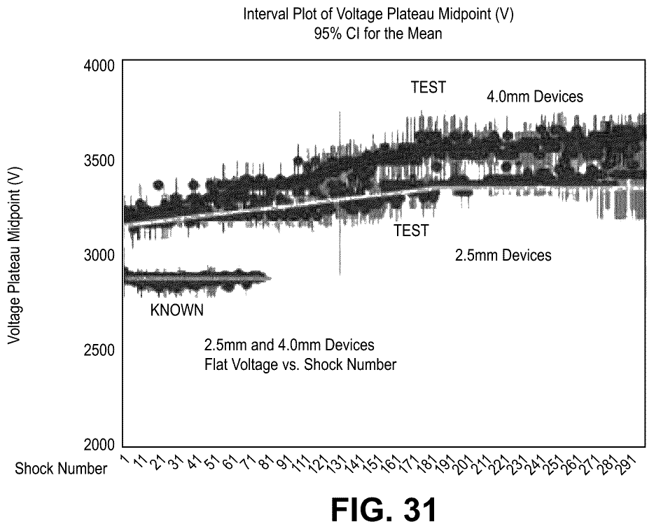

FIG. 31 presents a graphic comparison of known IVL devices comprising 2.5 mm and 4.0 mm OD balloons (KNOWN) with IVL devices according to the present disclosure and comprising 2.5 mm and 4.0 mm OD balloons (TEST).

The KNOWN devices permit 80 voltage pulses or shocks to be generated. The TEST devices generated 300 voltage pulses or shocks. During the comparative testing, for each voltage pulse, the voltage peak magnitude was obtained and plotted for each tested device. The KNOWN devices provide a relatively flat or constant voltage for each voltage pulse or shock number and begin at a lower voltage magnitude than the TEST devices. The TEST devices were operated and controlled in accordance with the disclosed embodiments herein.

In contrast, each of the 2.5 mm and 4.0 mm TEST devices begin at a higher voltage magnitude than the KNOWN devices. The 2.5 mm TEST device begins at a lower voltage than does the 4.0 mm device. As illustrated, both the 2.5 mm and the 4.0 mm TEST device voltage (lower data cluster) rise slowly over the generated voltage pulses, plateauing at approximately 180 pulses, thereafter remaining substantially flat or constant. In each case, the average voltage of the 4.0 mm TEST device is greater than that of the 2.5 mm TEST device.

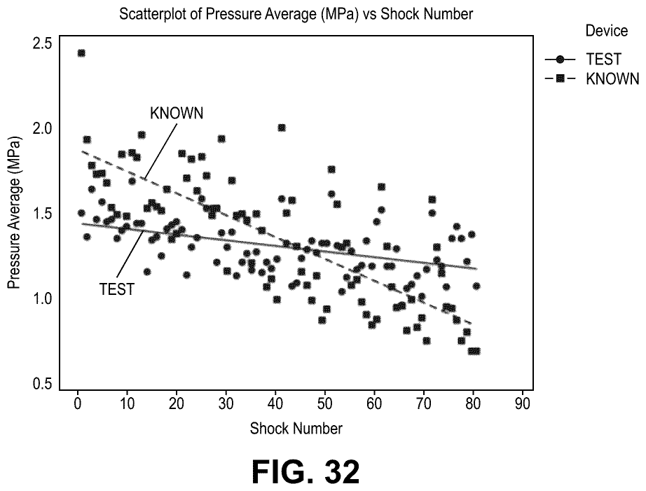

FIG. 32 illustrates a comparison of the TEST and KNOWN IVL devices and a subset of the data of FIG. 31, i.e., comparing the peak pressure outputs generated by the first 80 voltage pulses for each device type. Here, the voltage pulse (or shock number) is compared with the peak pressure generated during each voltage pulse. The KNOWN data with a constant voltage in each voltage pulse presents (dashed line) a relatively severe decrease in pressure output as the voltage pulses progress over time. In contrast, the TEST data (solid line) obtained using the voltage and pulse generation algorithm of FIG. 26, presents a pressure output line that decreases at a much smaller angle or slope. Thus, the pressure output of the TEST IVL devices provides a more stable or constant pressure output than the KNOWN IVL devices. The KNOWN IVL devices have a pronounced pressure output decay as the voltage pulses progress. More specifically, the TEST IVL devices provide a decrease in pressure output over 80 pulses that is less than 0.25 MPa.

The pressure output, as tested, was measured in-vitro using pressure sensors (hydrophones) positioned externally to the catheter balloon within the acoustic field generated by device pulse delivery. The device under test and hydrophones were immersed in de-gassed, deionized water maintained at approximately body temperature.

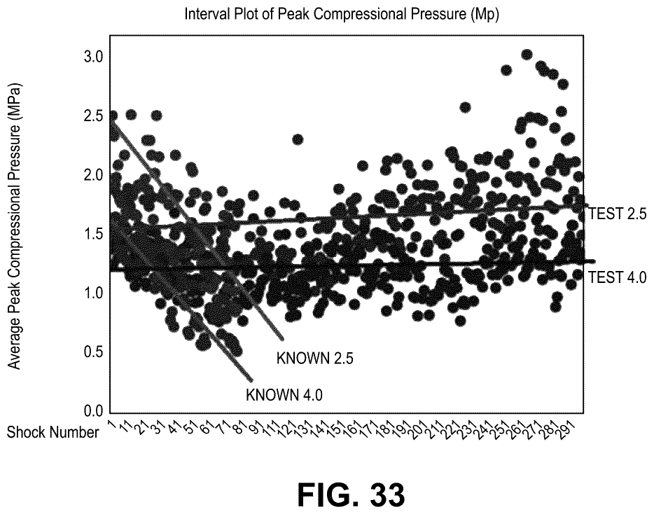

This concept is further demonstrated in FIG. 33, where the average pressure generated by 80 pulses of the KNOWN IVL devices (2.5 mm and 4.0 mm) with constant voltage magnitude is compared with the average pressure generated by the TEST IVL devices (2.5 mm and 4.0 mm) according to the voltage pulse and control method of FIG. 26.

As shown in FIG. 33, the KNOWN 2.5 mm and 4.0 mm devices both provide severely decreasing pressure output slope lines as the voltage pulses progress to 80 pulses. In contrast, the TEST 2.5 mm and 4.0 mm devices provide relatively flat, constant or stable pressure output slope lines as the voltage pulses progress to 300 pulses. Moreover, the slopes of the TEST pressure output lines appear to increase slightly as the voltage pulses progress which may be beneficial in cracking difficult calcified regions. Again, the KNOWN IVL devices have a pronounced and significant pressure output decay over 80 pulses, showing an approximate 60% decrease in pressure output. A significant pressure output decrease over 80 pulses may comprise more than a 10% decrease in pressure output as the therapeutic efficacy is reduced by a significant (more than 10%) amount. The TEST IVL devices do not have a pressure decay over 300 voltage pulses.

In summary, the IVL devices operated and controlled according to the present disclosure provide an increasing voltage to the voltage pulses until the upper voltage magnitude threshold is reached. Then, if the maximum number of voltage pulses has not been met, the voltage pulses may progress at the upper voltage magnitude threshold until 300, or more, voltage pulses (the predetermined maximum number of voltage pulses) have been executed, or the physical determines therapy is complete. This, as shown above, leads in certain embodiments, to constant and/or slightly increasing pressure output from each voltage pulse. The pressure output magnitudes, and associated slopes, may be manipulated in some embodiments by modifying the magnitude of each incremental increase in voltage. In some embodiments, the voltage magnitude may be incrementally increased as in FIG. 26. In others, the voltage magnitude may be increased incrementally for at least two series of voltage pulses, then held constant for one or more voltage pulses, then subsequent voltage pulse series may resume the incremental increase in magnitude. In other embodiments, the voltage magnitude may be decreased for one or more series of voltage pulses. All combinations of voltage increase, voltage decrease, and/or no change in voltage over a plurality of a series of voltage pulses in order to manipulate the resulting pressure output are within the scope of the present invention.

In addition, with reference to the above disclosure, various embodiments of the disclosure may provide a pressure output that is substantially the same for all balloon sizes, wherein the balloon sizes may be within a range from 2 mm to 4 mm outer diameter. In these embodiments, larger balloon sizes do not necessarily result in a lower pressure output than the pressure output of a relatively smaller balloon size.

The data of FIGS. 32 and 33 also demonstrate that IVL devices operated according to the present disclosure are also capable of stable operation and pressure output over a predetermined maximum number of voltage pulses comprising at least 300 voltage pulses. This is in contrast to the pronounced pressure decay of the KNOWN IVL devices over just 80 voltage pulses.

Moreover, the data of FIGS. 32 and 33 confirm that the IVL control system 22 shown in FIG. 1 is controllable such that the pressure output following an electrical arcing event between two spaced-apart electrodes can be controlled within upper and lower pressure magnitude thresholds or a pressure magnitude window. Further, the pressure output can be controlled using embodiments of the present disclosure in a pattern of increasing pressure over the procedure, decreasing pressure over the procedure, constant pressure over the procedure, and any combination thereof.

Examples of suitable processors may include one or more microprocessors, integrated circuits, system-on-a-chips (SoC), among others. Examples of suitable memory, may include one or more primary storage and/or non-primary storage (e.g., secondary, tertiary, etc. storage); permanent, semi-permanent, and/or temporary storage; and/or memory storage devices including but not limited to hard drives (e.g., magnetic, solid state), optical discs (e.g., CD-ROM, DVD-ROM), RAM (e.g., DRAM, SRAM, DRDRAM), ROM (e.g., PROM, EPROM, EEPROM, Flash EEPROM), volatile, and/or non-volatile memory; among others. Communication circuitry 58 includes components for facilitating processor operations, for example, suitable components may include transmitters, receivers, modulators, demodulators, filters, modems, analog/digital (AD or DA) converters, diodes, switches, operational amplifiers, and/or integrated circuits. In some embodiments, memory 26 may represent one or more memory devices operable for IVL therapy operation. For example, each memory (e.g., 154, 178) may be included as part of memory 26, shared, or isolated therefrom.

Within the present disclosure, consideration of a set of discharge electrodes has been discussed in the context of a pair of electrodes, one of which may serve as a cathode and the other of which may serve as an anode, at a given instance. However, the number of electrodes in a set may be greater than a pair, for example, including one or more cathodes communicating with one or more anodes. Additionally, devices, systems, and methods within the present disclosure may include more than one communicating group of electrodes, whether electrically arranged serially, parallel, or independently from each other.

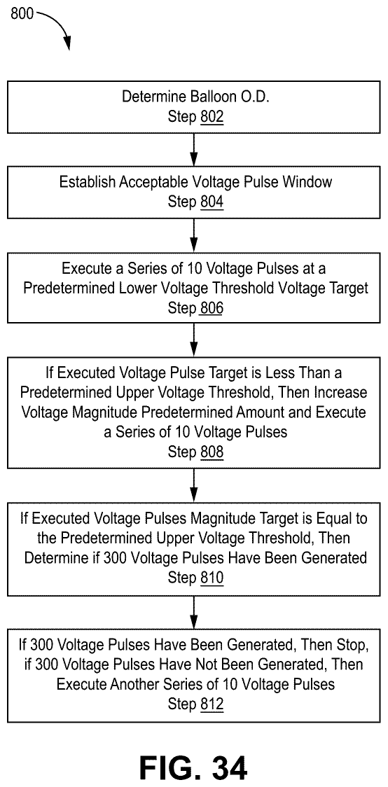

FIG. 34 provides an exemplary flowchart illustrating an exemplary method 800 of one embodiment of the present invention. Thus, step 802 provides for determination of the subject IVL device's balloon outer diameter or OD. This may be done with manual entry into the IVL control system discussed above. Alternatively, connecting the catheter with the IVL control system may provide automatic detection and determination of the balloon's OD. Step 804 provides for establishing an acceptable voltage pulse window, comprising as described above, predetermined lower and upper voltage magnitude thresholds which may be stored in the IVL control system. Step 806 provides for execution of a series of voltage pulses to be controlled and generated by the IVL control system, in an illustrative and exemplary case 10 pulses may be used, at the predetermined lower voltage magnitude threshold. Step 808 provides that if the IVL control system determines that the last executed series of voltage pulses was not executed at the predetermined upper voltage threshold target, then the IVL control system may instruct execution and generation of another series of voltage pulses. Step 810 provides that if the IVL control system determines that the last executed series of voltage pulses was executed at the predetermined upper voltage threshold target, then the IVL control system seeks to determine if the illustrative and exemplary predetermined maximum of 300 voltage pulses have been executed during the current therapy. If, according to step 812, 300 voltage pulses are determined to have been executed, the IVL control system stops the procedure, allowing no further voltage pulse generation. On the other hand, if 300 voltage pulses have not been executed, then the IVL control system instructs another series of voltage pulses to be executed and at the predetermined upper voltage threshold target magnitude.

In certain embodiments, the devices, systems and methods described herein may comprise a voltage pulsing frequency of 1 pulse/second, 2 pulses/second and/or 3 pulses/second. In some embodiments, the frequency or pulses/second generated by the described embodiments may be within the range of 1 to 5 pulses/second.

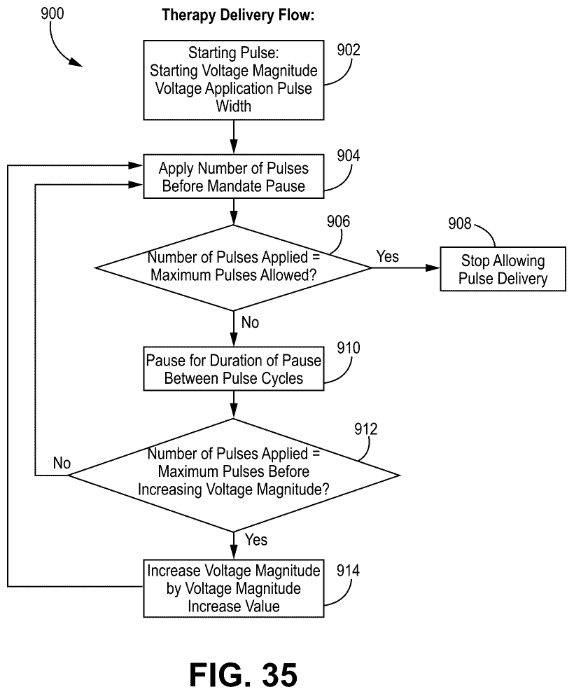

Turning now to FIG. 35, and with continued reference to FIGS. 1 and 26-34, some embodiments may comprise an IVL therapy flow 900 based on the control data comprising: the predetermined starting voltage; the upper voltage threshold; the width or duration of the generated and/or applied voltage pulse; the predetermined pause between adjacent voltage pulse series; the number of generated voltage pulses required before increasing voltage for subsequent voltage pulses; the magnitude of an increase in voltage of subsequent voltage pulses; the time gap between adjacent voltage pulses within a voltage pulse series; and the maximum number of voltage pulses. As discussed above, the control data may be stored in, and executed by, the control system 22. Alternatively, also as discussed above, the control data may be stored in a memory, processor, or an EPROM, wherein the control system 22 executes the therapy using the control data.

The executable IVL therapy flow 900 may be initiated in step 902 with generation of a starting voltage pulse and with the control data's predetermined duration or width of the voltage pulse. A predetermined number of voltage pulses (a series of voltage pulses) as provided by the control data may be executed in step 904, with a mandated or required pause of a predetermined duration before executing a subsequent or adjacent series of voltage pulses.

Next, in step 906, the number of generated voltage pulses is monitored or determined and compared with the control data's maximum number of voltage pulses allowed.

If, as in step 908, the maximum number of voltage pulses have been generated, then the control system 22 allows no further generation of voltage pulses. In some embodiments, additional voltage pulse generation may be allowed by swapping out the original catheter assembly 14 for a new catheter assembly, whereupon the number of voltage pulses generated restarts at zero.

If the maximum number of generated voltage pulses has not been reached, step 910 provides for the required pause between subsequent or adjacent series (or cycles) of the generated voltage pulses. The control data comprises the predetermined length or duration of the required pause between adjacent series of voltage pulses.

Following the mandated pause of step 910, step 912 determines if the total number of voltage pulses generated meets the control data's requirement for increasing the voltage magnitude for subsequent voltage pulses.

If the number of voltage pulses generated is insufficient to warrant an increase in voltage magnitude, then the operational flow returns to step 904 and subsequent steps as discussed above.

If, as in step 914, the number of voltage pulses generated is sufficient to warrant an increase in voltage magnitude, then the voltage magnitude is increased a predetermined amount for subsequent voltage pulses. From step 914, the operational flow returns to step 904 and subsequent steps as discussed above.

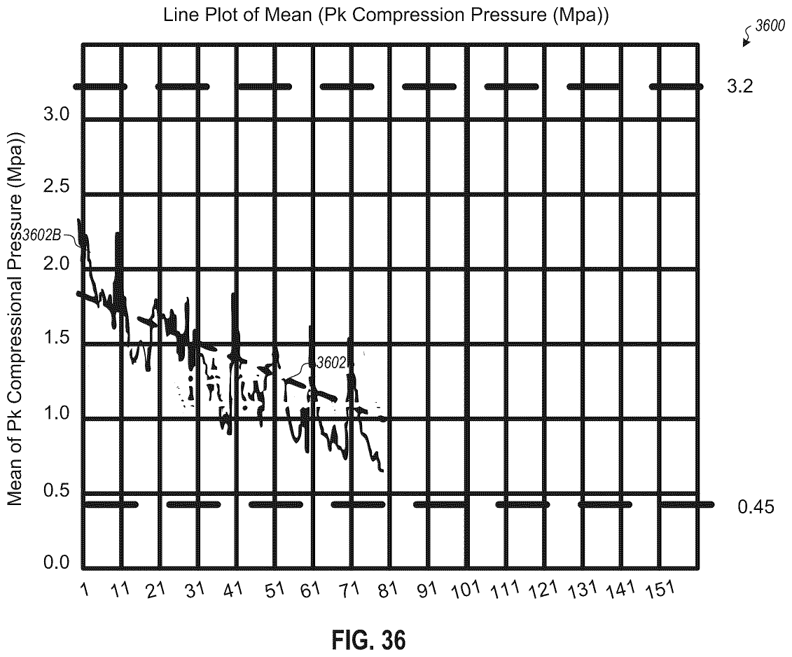

Referring now to FIGS. 36-43, various embodiments are illustrated that can be used to enhance balloon durability and/or enhance efficiency of treatment. This can be accomplished by identifying a target pressure profile for the balloon and adjusting applied voltages to achieve the target pressure profile. Adjusting voltages can include adjusting timing of voltage pulses and/or adjusting amplitude of voltage pulses.

One example of a KNOWN system is illustrated with reference to FIG. 36. FIG. 36 shows a pressure profiles by showing total number of pulses for a catheter assembly, including a balloon, graphed in comparison to compressional pressure. FIG. 36 illustrates a pressure profile illustrated as a line plot 3602 for a competitive KNOWN IVL device. In the illustrated example, the competitive KNOWN IVL device is of a type that maintains a constant amplitude of voltage pulses across electrodes and a constant rate of applying voltage pulses. The line plot 3602 is an average of compressional pressure over pulses, where the actual compressional pressure over pulses is shown by the curve 3602B.

Typically, there is a desire or requirement to keep the pressure between 3.2 MPa and 0.45 MPa as illustrated by the boundary lines in the graph 3600. As illustrated in the graph 3600, the competitive device is only capable of about 80 pulses before pressures drop below the 0.45 MPa limit. Thus, the competitive KNOWN device is only useful for about 80 pulses. Note that certain guidelines and/or regulations only allow 80 pulses per treatment location within a patient's vasculature. Thus, a single calcified lesion may be treated up to 80 pulses. Thus, in some instances, the balloon for the KNOWN competitive device may only be useful for treatment of a single calcified lesion. Further, as illustrated in the graph 3600, the average pressure declines over pulses meaning that, on average, as more pulses are delivered, less pressure is generated, and thus less energy can be delivered to the calcified lesion.

In contrast, certain embodiments illustrated herein are able to achieve an identified pressure profile by controlling physical characteristics of a catheter, magnitude of voltage pulses (including changes of magnitudes of voltage pulses), pulses between changes in magnitudes of pulses, pulse width, pulse frequency, cooldown duration, and/or pulses to cooldown. In some embodiments, having between 10 to 40 pulses between voltage magnitude changes can be used to achieve a particular pressure profile. Further still, in some embodiments, having at least 25 pulses between voltage magnitude changes can be used to achieve a particular pressure profile. In particular, an embodiment may be implemented with processes similar to that shown in FIG. 30 but with an selected numbers of pulses not just limited to that shown. FIG. 35 also illustrates an example process that may be used to implement embodiments illustrated. Further different voltage increases (or decreases) may be used to attempt to achieve a particular pressure profile.

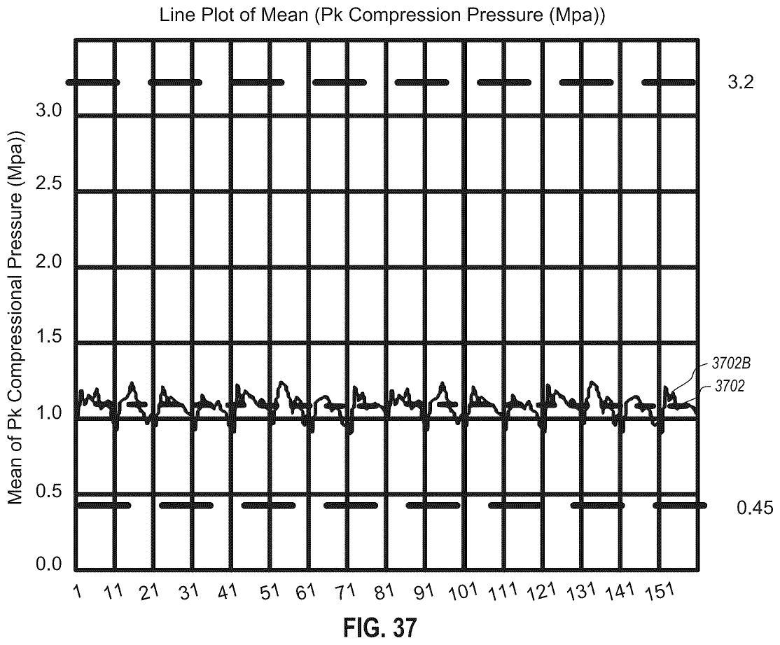

FIG. 37 illustrates an example where a substantially flat pressure profile is identified as represented by the line plot 3702 and achieved by generating pressures illustrated by the partially synthesized curve 3702B. Note that in this example, durability is improved over previous designs in that the catheter can now be used for well over the previous 80 pulses possible in previous catheter and IVL devices. Indeed, testing has shown that such embodiments may be implemented to withstand at least 200 pulses. Even more specifically, statistical analysis has shown that 95% of catheters implemented with the configuration illustrated in FIG. 1 would have a pulse durability of 218 pulses. In particular, testing was done with 25 sample units and the lowest observed number of pulses prior to failure was 320 due to emitter failure, whereas the lowest failure due to balloon bursting was observed after pulse 480.

Testing was performed using balloons constructed of a single layer of Vestamid ML21 and having a double-wall thickness in a mid-region of the balloon, e.g., adjacent electrodes 18) of 0.0018″+/−0.0005″ [providing a nominal wall thickness (see dimension 222 in FIG. 7A) of about 0.0009″]. The nominal wall thickness at a proximal tail of the balloon was within the range from 0.0012″ to 0.002″. As used in the art, a “double-wall thickness” means twice a nominal wall thickness and can be measured non-destructively on a folded balloon by observing a thickness of the folded balloon, for example, by radially collapsing a balloon to bring two opposed layers together and measuring the resulting thickness of the two opposed layers. Testing was performed starting the voltage pulses to have a magnitude of 2700 V, increasing the voltage by 25 V every 40 pulses, having pulse widths of 25 microseconds, having a pulse frequency of 2 Hz, having a cooldown period duration of 10 seconds, and having a cooldown period after every 10 pulses.

Various balloon configurations and tests were performed according to the following table:

TABLE 3

| | 2.5 mm × | 2.5 mm × | 3.0 mm × | 3.5 mm × | 4.0 mm × | 4.0 mm × |
|---|---|---|---|---|---|---|
| Parameter | 12 | 20 | 12 | 12 | 12 | 12 |
| Starting Voltage | 2700 | 2700 | 2850 | 3000 | 3250 | 3250 |
| Setpoint (V) | | | | | | |
| Pulses to Increase | 40 | 40 | 10 | 10 | 10 | 10 |
| Voltage Setpoint | | | | | | |
| Voltage Step Size (V) | 25 | 25 | 25 | 25 | 25 | 25 |
| Pulse Width | 25 | 25 | 25 | 25 | 25 | 25 |
| (microseconds) | | | | | | |
| Pulse Frequency (Hz) | 2 | 2 | 2 | 2 | 2 | 2 |
| Cooldown Duration | 10 | 10 | 10 | 10 | 10 | 10 |
| (seconds) | | | | | | |
| Pulses to Cooldown | 10 | 10 | 10 | 10 | 10 | 10 |

Testing using these parameters resulted in durability for balloons of at least 160 pulses without failure and flatter median pressure profile than previous known systems.

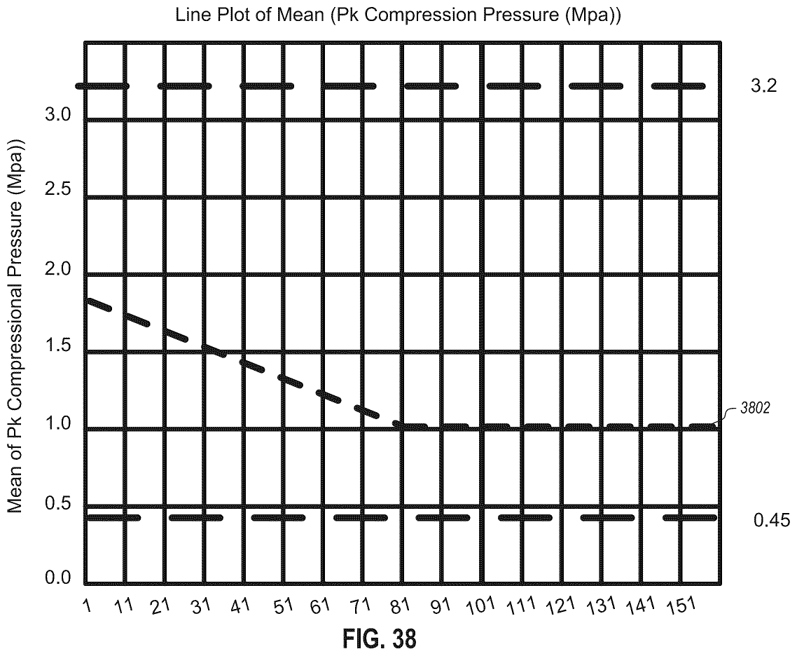

Referring now to FIG. 38, a declining-flat pressure profile is illustrated by the line plot 3802. The declining flat pressure profile specifies a higher initial pressure (i.e., higher than certain subsequent pressures), declines from the higher initial pressure over a first set of pulses to be delivered, then remains flat over a second set comprising the remaining pulses to be delivered. This pressure profile has the benefit of higher initial pressures which deliver the most energy to a treatment site (e.g., a calcified lesion) in the first pulses. The pressure profile flattens out to provide consistent additional therapy if additional pulses are needed. Reducing pressure reduces total energy delivered and reduces total stress on balloon material, improving durability over the life of the balloon. This profile can be achieved, in some embodiments by intentionally not varying (or minimizing variance) of pulse magnitude over pulses in the declining portion of the profile, and then increasing voltage magnitudes over the flat portion of the profile. For example, for a 2.5 mm OD 12 mm balloon, voltage magnitude may start at 2750 Volts and be held constant for 80 pulses. Voltage magnitude may then be increased on subsequent pulses, such as by increasing 25 Volts every 40 pulses.

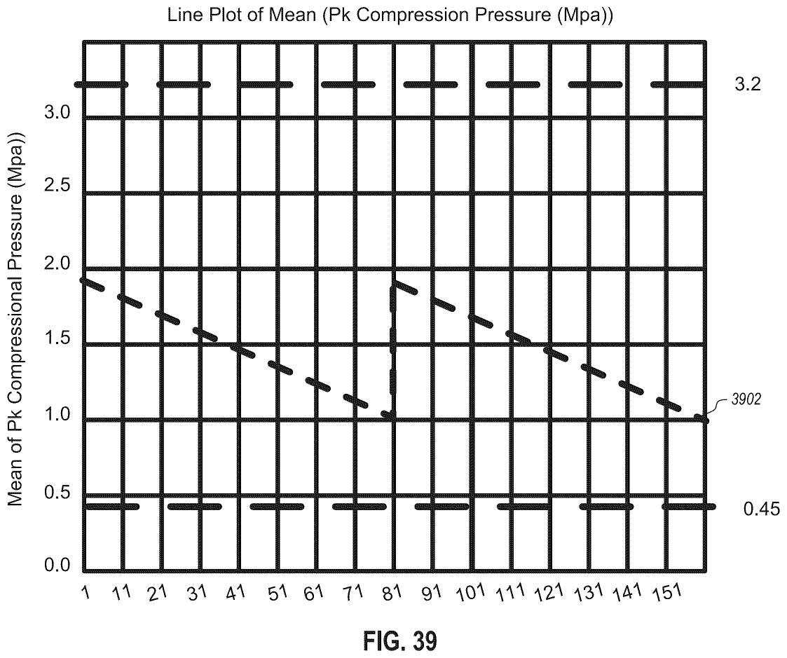

Referring now to FIG. 39, a sawtooth declining pressure profile is illustrated by the curve 3902. In the illustrated example, the sawtooth declining pressure profile starts at a higher initial pressure (i.e., higher than certain subsequent pressures) and declines until pulse 80. Pulse 81 starts over at the higher initial pressure and declines to pulse 160. Note that other pulse numbers may be used and the example illustrated here is for illustrative purposes only. The sawtooth declining pressure profile has higher initial pressures to provide the most energy at a treatment site in the first pulses. Clinical indications limit therapy to 80 pulses per segment of vessel treated. Increasing pressure again at pulse 81 allows for two segments to be treated with a single balloon, each with matching energy delivery. Reducing pressure reduces total energy delivered and reduces total stress on balloon material, improving durability over the life of the balloon. This profile can be accomplished, in some embodiments, by not varying (or minimizing variance) of pulse magnitude over pulses in the first declining portion of the profile, and then increasing voltage magnitudes at the beginning of the second declining portion of the profile (e.g., by about 100 Volts), but not varying (or minimizing variance) of pulse magnitude over pulses in the second declining portion of the profile.

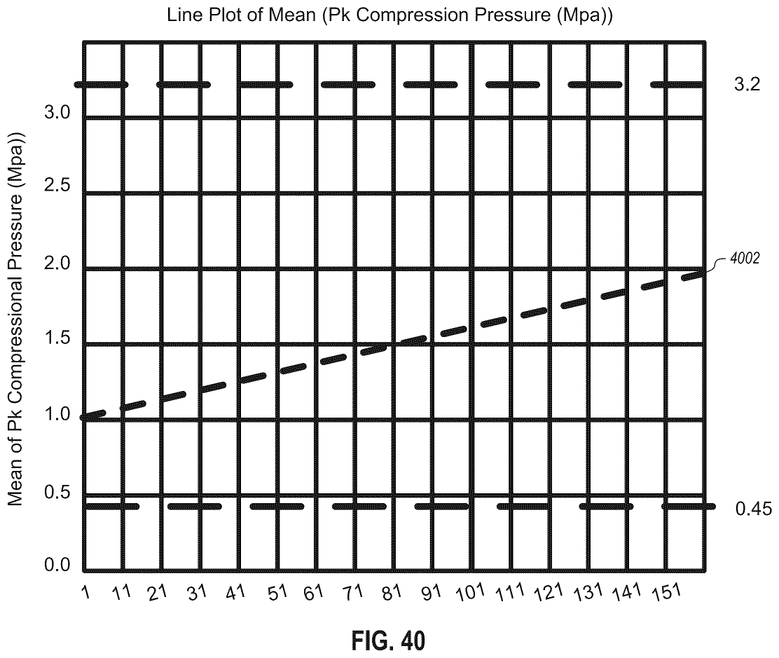

Referring now to FIG. 40, an increasing pressure profile is illustrated by the curve 4002. The increasing pressure profile starts at a lower initial pressure, increasing over pulses delivered. A lower initial pressure may be effective therapy in some lesions. Increasing pressure with each pulse can be used to provide additional therapy for more difficult lesions. Starting at a lower pressure reduces total energy delivered and reduces total stress on the balloon material, improving durability over the life of the balloon. The increasing pressure profile may be accomplished by increasing voltage over pulses. For example, using a 2.5 mm OD 12 mm balloon, voltage may be increased by 25 volts every 15 pulses.

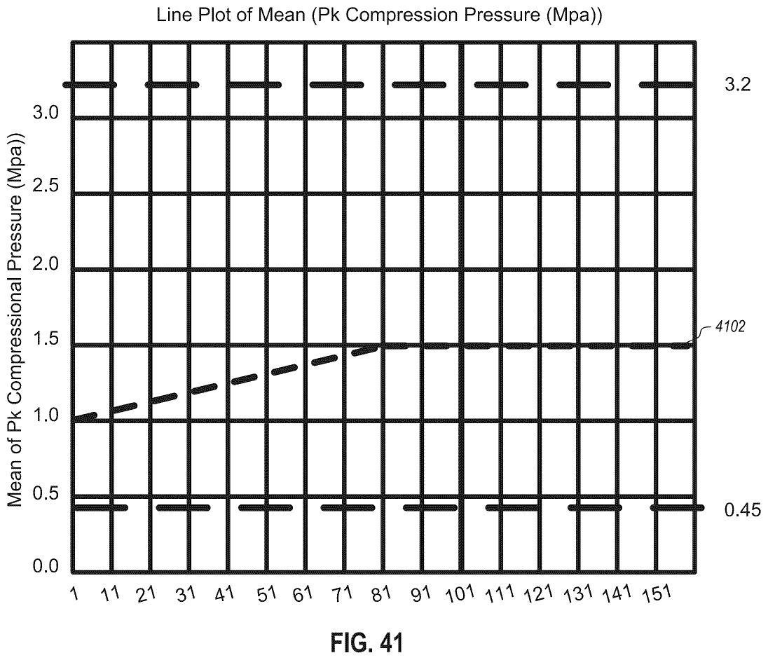

Referring now to FIG. 41, an increasing-flat pressure profile is illustrated by the curve 4102. The increasing-flat pressure profile is a profile that starts at a lower initial pressure (i.e., lower than certain subsequent pressures), increasing over a certain number of pulses delivered, then remains flat for the remaining pulses delivered. Lower initial pressure may be effective therapy in some lesions. Increasing pressure with each pulse is done to provide additional therapy for more difficult lesions. The pressure profile flattens out to provide consistent additional therapy if additional pulses are needed. Starting at a lower pressure reduces total energy delivered and reduces total stress on balloon material, improving durability over the life of the balloon. The increasing-flat pressure profile may be accomplished by increasing voltage at a first rate and/or amount followed by increasing voltage at a second rate/and or amount. For example, assuming a 2.5 mm OD 12 mm balloon, the increasing portion may be implemented by increasing voltage by 25 Volts every 10 pulses while the flat portion is implemented by increasing voltage by 25 Volts every 40 pulses. Alternatively, the increasing portion may be implemented by increasing voltage by 30 Volts every 20 pulses while the flat portion is implemented by increasing voltage by 25 Volts every 25 pulses.

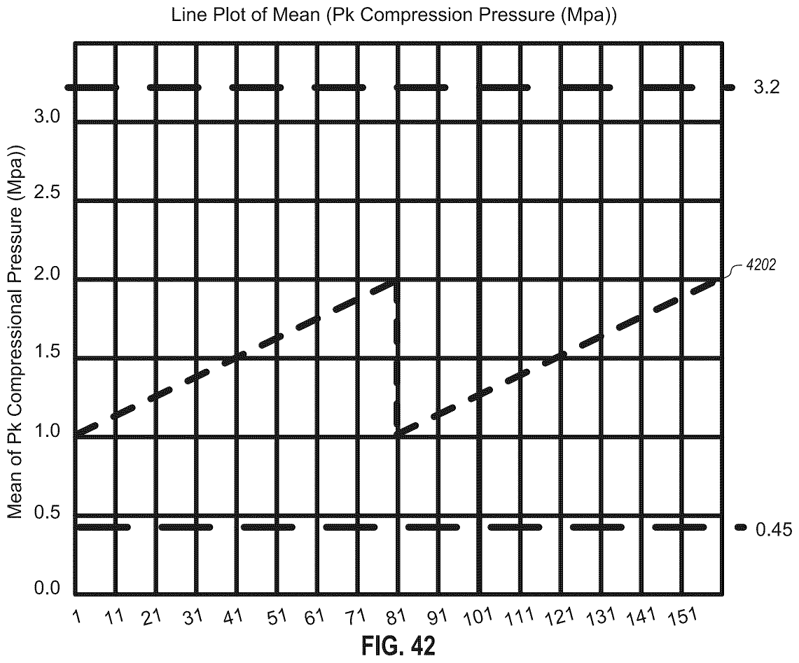

Referring now to FIG. 42, a sawtooth increasing pressure profile is illustrated by the curve 4202. In the illustrated example, the sawtooth increasing pressure profile starts at a lower initial pressure and increases until pulse 80. Pulse 81 starts over at the lower initial pressure and increases until pulse 160. The pulse numbers here are for illustration purposes only and alternative pulse numbers may be used in alternative embodiments. Lower initial pressure may be effective therapy in some lesions. Clinical indications limit therapy to 80 pulses per segment of vessel treated, decreasing pressure again at pulse 81 allows for two segments to be treated with a single balloon, each with matching energy delivery. Starting at a lower pressure reduces total energy delivered and reduces total stress on balloon material, improving durability over the life of the balloon. The sawtooth increasing pressure profile may be accomplished by increasing voltage at a first rate and/or amount followed by decreasing voltage by a second amount followed by increasing voltage at a third rate/and or amount. For example, assuming a 2.5 mm OD 12 mm balloon, the first increasing portion may be implemented by increasing voltage by 25 Volts every 10 pulses followed by a decrease of 75 Volts to begin the second increasing portion, followed by increasing voltage by 25 Volts every 10 pulses to achieve the second increasing portion.

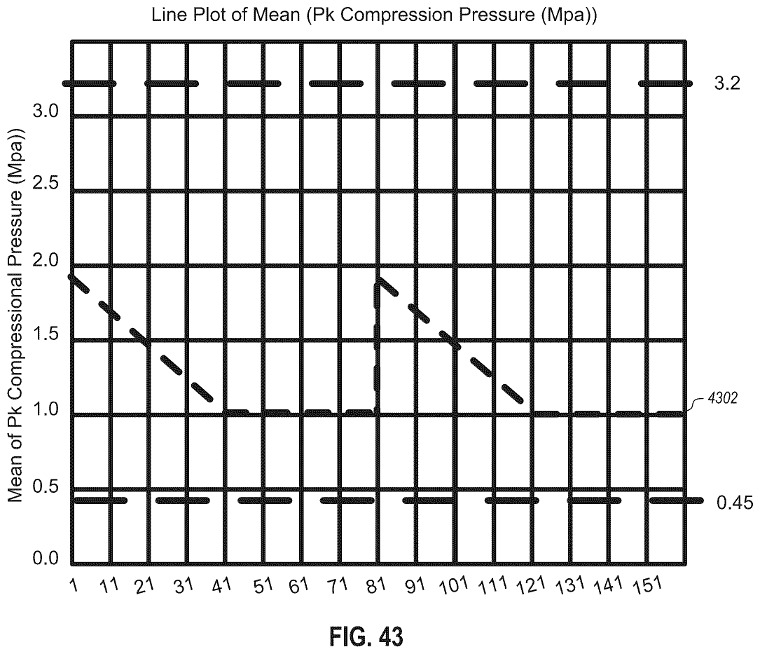

Referring now to FIG. 43 a decreasing-flat-decreasing-flat pressure profile is illustrated by the curve 4302. A decreasing-flat-decreasing-flat pressure profile delivers high pressure near the beginning, but then subsequently levels off to preserve the life of the balloon. This is repeated for a second treatment location. The decreasing-flat-decreasing-flat pressure profile has the benefit of higher initial pressures, at multiple vein sites, which deliver the most energy to a treatment site (e.g., a calcified lesion) in the first pulses at each site. The pressure profile flattens out to provide consistent additional therapy if additional pulses are needed. Reducing pressure reduces total energy delivered and reduces total stress on balloon material, improving durability over the life of the balloon. This profile can be achieved, in some embodiments by intentionally not varying (or minimizing variance), or even reducing, pulse voltage magnitude over pulses in the first declining portion of the profile, and then increasing voltage magnitudes over the flat portion of the profile, then more dramatically increasing voltage magnitudes at the peak of the second declining portion, then by intentionally not varying (or minimizing variance), or even reducing, pulse voltage magnitude over pulses in the second declining portion of the profile, then increasing voltage magnitudes over the second flat portion.

Various other combinations of pulses and voltage increases may be used to achieve the various profiles illustrated above. Further various sizes and types of balloons may be used.

In some embodiments, the balloon is constructed from a polymeric material with a double-wall thickness of 0.0023″ or less.

In some embodiment, when inflated, the balloon has a radius from an axis of the balloon of approximately 2.5 mm and a length of 20 mm or less.

In some embodiment, when inflated, the balloon has a radius from an axis of the balloon of approximately 3.0 mm and a length of 20 mm or less.

In some embodiment, when inflated, the balloon has a radius from an axis of the balloon of approximately 3.5 mm and a length of 20 mm or less.

In some embodiment, when inflated, the balloon has a radius from an axis of the balloon of approximately 4.0 mm and a length of 20 mm or less.

In some embodiment, the balloon material is a Nylon material.

In some embodiment, the balloon material is Vestamid.

In some embodiments, each catheter can be programmed with metadata retrievable by an electric pulse generation system to which the catheter will be connected. For example, such information may be stored on a memory incorporated into the catheter. In one example, such information may include a specific identifier for the catheter. The electric pulse generation system may be able to determine from the specific identifier characteristics about the catheter, such as outer diameter, length, balloon material, balloon wall thickness, balloon deformation characteristics, etc. In an additional or alternative example, balloon characteristics may be stored on the memory incorporated into the catheter. FIG. 22 shows an example EPROM. This EPROM could be used to store catheter information. Alternatively, or additionally, the EPROM could store the parameters/instructions for how to adjust voltage with each pulse delivered to achieve predetermined pressure profiles.

Configuration 1. An intravascular lithotripsy (“IVL”) system including a catheter assembly, the system comprising: at least one set of electrodes for arrangement within a body lumen while disposed within an inflatable balloon or enclosure; an electric pulse generation system for providing electrical energy to the at least one set of electrodes to generate spark for IVL therapy, the electric pulse generation system including an IVL control system comprising a processor for executing instructions based at least in part on a set of control data stored on a memory, and circuitry adapted for communication of signals based on operation of the processor, the IVL control system configured to: identify a pressure profile identifying target pressures for in the inflatable balloon or enclosure over a course of IVL therapy; based on the identified profile, identify a series of voltage pulses that when generated at the at least one set of electrodes would generate pressures having a mean over the course of therapy that approximates the pressure profile wherein the series of pulses comprises at least one sub series of pulses comprising at least 25 sequential pulses of a same voltage magnitude, after which the voltage magnitude is changed for subsequent voltage pulses; and generate the series of voltage pulses.

Configuration 2. The system of configuration 1, wherein the IVL control system is further configured to increase the magnitude of the subsequent voltage pulses by a predetermined amount of about 25 Volts.

Configuration 3. The system of any of configurations 1-2, wherein the at least one sub series of pulses comprises at least 40 sequential pulses of a same voltage magnitude.

Configuration 4. The system of any of configurations 1-3, wherein the pressure profile comprises a substantially flat pressure profile where the pressure profile remains constant over the series of voltage pulses.

Configuration 5. The system of any of configurations 1-4, wherein the pressure profile comprises a declining-flat pressure profile specifying an initial pressure that declines over subsequent voltage pulses until a predetermined number of subsequent voltage pulses, after which the pressure is substantially flat.

Configuration 6. The system of any of configurations 1-5, wherein the pressure profile comprises a sawtooth declining pressure profile specifying an initial pressure that declines over subsequent voltage pulses follow by an increased pressure which declines over subsequent voltage pulses.

Configuration 7. The system of any of configurations 1-6, wherein the pressure profile comprises a increasing pressure profile specifying an initial pressure followed by increasing pressure over subsequent voltage pulses.

Configuration 8. The system of any of configurations 1-7, wherein the pressure profile comprises an increasing-flat pressure profile specifying an initial pressure followed by increasing pressure over subsequent voltage pulses until a predetermined number of voltage pulses after which the pressure is substantially flat.

Configuration 9. The system of any of configurations 1-8, wherein the pressure profile comprises a sawtooth increasing pressure profile specifying an initial pressure followed by increasing pressure over subsequent voltage pulses until a predetermined number of voltage pulses after which pressure is decreased followed by increasing pressure over subsequent voltage pulses.

Configuration 10. The system of any of configurations 1-9, wherein the pressure profile comprises a decreasing-flat-decreasing-flat pressure profile specifying a first initial pressure that declines over subsequent voltage pulses until a first predetermined number of subsequent voltage pulses, after which the pressure is substantially flat until a second predetermined number of subsequent voltage pulses after which pressure is increased to a second initial pressure that declines over subsequent voltage pulses until a third predetermined number of subsequent voltage pulses, after which the pressure is substantially flat.

Configuration 11. The system of any of configurations 1-10, wherein the catheter assembly comprises an EPROM storing at least one of parameters or instructions for how to adjust voltage with each pulse delivered to achieve the identified pressure profile.

Configuration 12. A method for generating and controlling voltage pulses using the system of any of configurations 1-11.

Configuration 13. A method of performing intravascular coronary lithotripsy (“IVL”) using a catheter assembly having an axis, the method comprising: delivering a set of voltage pulses in a balloon, inflated by fluid, the balloon being sized and shaped for use in a coronary vessel; and wherein the total of the set of voltage pulses capable of being provided by the catheter is more than 120 voltage pulses using the same balloon.

Configuration 14. The method of configuration 13, comprising causing 10 to 40 sequential voltage pulses of a substantially same voltage magnitude, after which the voltage magnitude is changed for subsequent voltage pulses.

Configuration 15. The method of any of configurations 13-14, comprising causing 25 sequential voltage pulses of a substantially same voltage magnitude, after which the voltage magnitude is changed for subsequent voltage pulses.

Configuration 16. The method of any of configurations 13-15, wherein the set of voltage pulses is at least 160 pulses.

Configuration 17. The method of any of configurations 13-16, wherein the set of voltage pulses cause a mean pressure within the balloon over the course of the IVL that approximates a substantially flat pressure profile.

Configuration 18. The method of any of configurations 13-17, wherein the set of voltage pulses cause a mean pressure within the balloon over the course of the IVL that approximates an increasing pressure profile.

Configuration 19. The method of any of configurations 13-18, wherein the set of voltage pulses cause a mean pressure within the balloon over the course of the IVL that approximates a declining-flat pressure profile.

Configuration 20. The method of any of configurations 13-19, wherein the set of voltage pulses cause a mean pressure within the balloon over the course of the IVL that approximates a sawtooth declining pressure profile.

Configuration 21. The method of any of configurations 13-20, wherein the set of voltage pulses cause a mean pressure within the balloon over the course of the IVL that approximates a sawtooth increasing pressure profile.

Configuration 22. The method of any of configurations 13-21, wherein the set of voltage pulses cause a mean pressure within the balloon over the course of the IVL that approximates a decreasing-flat-decreasing-flat pressure profile.

Configuration 23. The method of any of configurations 13-22, further comprising advancing the balloon to a first therapy location to facilitate delivering a first subset of voltage pulses from among the set of voltage pulses and repositioning the balloon to a second therapy location to facilitate delivering a second subset of voltage pulses from among the set of voltage pulses.

Configuration 24. The method of any of configurations 13-23, wherein the balloon is constructed from a polymeric material with a double-wall thickness of 0.0023″ or less.

Configuration 25. The method of any of configurations 13-24 wherein, when inflated, the balloon has a radius from an axis of the balloon of approximately 2.5 mm and a length of 20 mm or less.

Configuration 26. The method of any of configurations 13-25 wherein, when inflated, the balloon has a radius from an axis of the balloon of approximately 3.0 mm and a length of 20 mm or less.

Configuration 27. The method of any of configurations 13-26 wherein, when inflated, the balloon has a radius from an axis of the balloon of approximately 3.5 mm and a length of 20 mm or less.

Configuration 28. The method of any of configurations 13-27 wherein, when inflated, the balloon has a radius from an axis of the balloon of approximately 4.0 mm and a length of 20 mm or less.

Configuration 29. The method of any of configurations 13-28 wherein, the polymeric material is a Nylon material.

Configuration 30. The method of any of configurations 13-29 wherein, the polymeric material is Vestamid.

Configuration 31. The method of any of configurations 13-30 wherein the at least one set of electrodes comprises two pairs of electrodes, each pair of electrodes having a gap, and each pulse provides a spark in the gap between both of the pairs of electrodes.

Configuration 32. An intravascular lithotripsy (“IVL”) system including a catheter assembly, the system comprising:
at least one set of electrodes for arrangement within a coronary vessel lumen while disposed within an inflatable balloon;
an electric pulse generation system for providing electrical energy to the at least one set of electrodes to generate spark for IVL therapy, the electric pulse generation system including an IVL control system; and
wherein the combination of the at least one set of electrodes for arrangement within a body lumen while disposed within an inflatable balloon and the electric pulse generation system are configured to provide more than 120 voltage pulses.

Configuration 33. The system of any of configuration 32, wherein the control system is configured to cause between 10 and 40 sequential voltage pulses of a substantially same voltage magnitude, after which the voltage magnitude is changed for subsequent voltage pulses.

Configuration 34. The system of any of configurations 32-33, wherein the control system is configured to cause at least 25 sequential voltage pulses of a substantially same voltage magnitude, after which the voltage magnitude is changed for subsequent voltage pulses.

Configuration 35. The system of any of configurations 32-34, wherein the combination of the at least one set of electrodes for arrangement within a body lumen while disposed within an inflatable balloon and the electric pulse generation system are configured to provide voltage pulses cause a mean pressure within the balloon over the course of IVL that approximates a substantially flat pressure profile.

Configuration 36. The system of any of configurations 32-35, wherein the combination of the at least one set of electrodes for arrangement within a body lumen while disposed within an inflatable balloon and the electric pulse generation system are configured to provide voltage pulses cause a mean pressure within the balloon over the course of IVL that approximates an increasing pressure profile.

Configuration 37. The system of any of configurations 32-36, wherein the combination of the at least one set of electrodes for arrangement within a body lumen while disposed within an inflatable balloon and the electric pulse generation system are configured to provide voltage pulses cause a mean pressure within the balloon over the course of IVL that approximates a declining-flat pressure profile.

Configuration 38. The system of any of configurations 32-37, wherein the combination of the at least one set of electrodes for arrangement within a body lumen while disposed within an inflatable balloon and the electric pulse generation system are configured to provide voltage pulses cause a mean pressure within the balloon over the course of IVL that approximates a sawtooth declining pressure profile.

Configuration 39. The system of any of configurations 32-38, wherein the combination of the at least one set of electrodes for arrangement within a body lumen while disposed within an inflatable balloon and the electric pulse generation system are configured to provide voltage pulses cause a mean pressure within the balloon over the course of IVL that approximates a sawtooth increasing pressure profile.

Configuration 40. The system of any of configurations 32-39, wherein the combination of the at least one set of electrodes for arrangement within a body lumen while disposed within an inflatable balloon and the electric pulse generation system are configured to provide voltage pulses cause a mean pressure within the balloon over the course of IVL that approximates a decreasing-flat-decreasing-flat pressure profile.

Configuration 41. The system of any of configurations 32-40, wherein the balloon is constructed from a polymeric material with a double-wall thickness of 0.0023″ or less.

Configuration 42. The system of any of configurations 32-41 wherein, when inflated, the balloon has a radius from an axis of the balloon of approximately 2.5 mm and a length of 20 mm or less.

Configuration 43. The system of any of configurations 32-42 wherein, when inflated, the balloon has a radius from an axis of the balloon of approximately 3.0 mm and a length of 20 mm or less.

Configuration 44. The system of any of configurations 32-43 wherein, when inflated, the balloon has a radius from an axis of the balloon of approximately 3.5 mm and a length of 20 mm or less.

Configuration 45. The system of any of configurations 32-44 wherein, when inflated, the balloon has a radius from an axis of the balloon of approximately 4.0 mm and a length of 20 mm or less.

Configuration 46. The system of any of configurations 32-45 wherein, the polymeric material is a Nylon material.

Configuration 47. The system of any of configurations 32-46 wherein, the polymeric material is Vestamid.

Configuration 48. The system of any of configurations 32-47 wherein the at least one set of electrodes comprises two pairs of electrodes, each pair of electrodes having a gap, and each pulse provides a spark in the gap between both of the pairs of electrodes.

The description of devices, systems, and method and related applications as set forth herein is illustrative and is not intended to limit the scope of the invention. Features of various embodiments may be combined with other embodiments within the contemplation of this invention. Variations and modifications of the embodiments disclosed herein are possible, and practical alternatives to and equivalents of the various elements of the embodiments would be understood to those of ordinary skill in the art upon study of this patent document. These and other variations and modifications of the embodiments disclosed herein may be made without departing from the scope and spirit of the invention.

We claim:

1. An intravascular lithotripsy (“IVL”) system including a catheter assembly, the system comprising:
at least one set of electrodes for arrangement within a body lumen while disposed within an inflatable balloon or enclosure;
an electric pulse generation system for providing electrical energy to the at least one set of electrodes to generate spark for IVL therapy, the electric pulse generation system including an IVL control system comprising a processor for executing instructions based at least in part on a set of control data stored on a memory, and circuitry adapted for communication of signals based on operation of the processor, the IVL control system configured to:
identify a pressure profile identifying target pressures for in the inflatable balloon or enclosure over a course of IVL therapy;
based on the identified profile, identify a series of voltage pulses that when generated at the at least one set of electrodes would generate pressures having a mean over the course of therapy that approximates the pressure profile wherein the series of pulses comprises at least one sub series of pulses comprising at least 25 sequential pulses of a same voltage magnitude, after which the voltage magnitude is changed for subsequent voltage pulses; and
generate the series of voltage pulses.

2. The system of claim 1, wherein the IVL control system is further configured to increase the magnitude of the subsequent voltage pulses by a predetermined amount of about 25 Volts.

3. The system of claim 1, wherein the at least one sub series of pulses comprises at least 40 sequential pulses of a same voltage magnitude.

4. The system of claim 1, wherein the pressure profile comprises a substantially flat pressure profile where the pressure profile remains constant over the series of voltage pulses.

5. The system of claim 1, wherein the pressure profile comprises a declining-flat pressure profile specifying an initial pressure that declines over subsequent voltage pulses until a predetermined number of subsequent voltage pulses, after which the pressure is substantially flat.

6. The system of claim 1, wherein the pressure profile comprises a sawtooth declining pressure profile specifying an initial pressure that declines over subsequent voltage pulses follow by an increased pressure which declines over subsequent voltage pulses.

7. The system of claim 1, wherein the pressure profile comprises a increasing pressure profile specifying an initial pressure followed by increasing pressure over subsequent voltage pulses.

8. The system of claim 1, wherein the pressure profile comprises an increasing-flat pressure profile specifying an initial pressure followed by increasing pressure over subsequent voltage pulses until a predetermined number of voltage pulses after which the pressure is substantially flat.

9. The system of claim 1, wherein the pressure profile comprises a sawtooth increasing pressure profile specifying an initial pressure followed by increasing pressure over subsequent voltage pulses until a predetermined number of voltage pulses after which pressure is decreased followed by increasing pressure over subsequent voltage pulses.

10. The system of claim 1, wherein the pressure profile comprises a decreasing-flat-decreasing-flat pressure profile specifying a first initial pressure that declines over subsequent voltage pulses until a first predetermined number of subsequent voltage pulses, after which the pressure is substantially flat until a second predetermined number of subsequent voltage pulses after which pressure is increased to a second initial pressure that declines over subsequent voltage pulses until a third predetermined number of subsequent voltage pulses, after which the pressure is substantially flat.

11. The system of claim 1, wherein the catheter assembly comprises an EPROM storing at least one of parameters or instructions for how to adjust voltage with each pulse delivered to achieve the identified pressure profile.

12. A method for generating and controlling voltage pulses using the system of claim 1.

13. A method of performing intravascular coronary lithotripsy (“IVL”) using a catheter assembly having an axis, the method comprising:
delivering a set of voltage pulses in a balloon, inflated by fluid, the balloon being sized and shaped for use in a coronary vessel; and
wherein the total of the set of voltage pulses capable of being provided by the catheter is more than 120 voltage pulses using the same balloon.

14. The method of claim 13, comprising causing 10 to 40 sequential voltage pulses of a substantially same voltage magnitude, after which the voltage magnitude is changed for subsequent voltage pulses.

15. The method of claim 13, comprising causing 25 sequential voltage pulses of a substantially same voltage magnitude, after which the voltage magnitude is changed for subsequent voltage pulses.

16. The method of claim 13, wherein the set of voltage pulses is at least 160 pulses.

17. The method of claim 13, wherein the set of voltage pulses cause a mean pressure within the balloon over the course of the IVL that approximates a substantially flat pressure profile.

18. The method of claim 13, wherein the set of voltage pulses cause a mean pressure within the balloon over the course of the IVL that approximates an increasing pressure profile.

19. The method of claim 13, wherein the set of voltage pulses cause a mean pressure within the balloon over the course of the IVL that approximates a declining-flat pressure profile.

20. The method of claim 13, wherein the set of voltage pulses cause a mean pressure within the balloon over the course of the IVL that approximates a sawtooth declining pressure profile.

21. The method of claim 13, wherein the set of voltage pulses cause a mean pressure within the balloon over the course of the IVL that approximates a sawtooth increasing pressure profile.

22. The method of claim 13, wherein the set of voltage pulses cause a mean pressure within the balloon over the course of the IVL that approximates a decreasing-flat-decreasing-flat pressure profile.

23. The method of claim 13, further comprising advancing the balloon to a first therapy location to facilitate delivering a first subset of voltage pulses from among the set of voltage pulses and repositioning the balloon to a second therapy location to facilitate delivering a second subset of voltage pulses from among the set of voltage pulses.

24. The method of claim 13, wherein the balloon is constructed from a polymeric material with a double-wall thickness of 0.0023″ or less.

25. The method of claim 13 wherein, when inflated, the balloon has a radius from an axis of the balloon of approximately 2.5 mm and a length of 20 mm or less.

26. The method of claim 13 wherein, when inflated, the balloon has a radius from an axis of the balloon of approximately 3.0 mm and a length of 20 mm or less.

27. The method of claim 13 wherein, when inflated, the balloon has a radius from an axis of the balloon of approximately 3.5 mm and a length of 20 mm or less.

28. The method of claim 13 wherein, when inflated, the balloon has a radius from an axis of the balloon of approximately 4.0 mm and a length of 20 mm or less.

29. The method of claim 24 wherein, the polymeric material is a Nylon material.

30. The method of claim 24 wherein, the polymeric material is Vestamid.

31. The method of claim 13 wherein the at least one set of electrodes comprises two pairs of electrodes, each pair of electrodes having a gap, and each pulse provides a spark in the gap between both of the pairs of electrodes.

32. An intravascular lithotripsy (“IVL”) system including a catheter assembly, the system comprising:
at least one set of electrodes for arrangement within a coronary vessel lumen while disposed within an inflatable balloon;
an electric pulse generation system for providing electrical energy to the at least one set of electrodes to generate spark for IVL therapy, the electric pulse generation system including an IVL control system; and
wherein the combination of the at least one set of electrodes for arrangement within a body lumen while disposed within an inflatable balloon and the electric pulse generation system are configured to provide more than 120 voltage pulses.

33. The system of claim 32, wherein the control system is configured to cause between 10 and 40 sequential voltage pulses of a substantially same voltage magnitude, after which the voltage magnitude is changed for subsequent voltage pulses.

34. The system of claim 32, wherein the control system is configured to cause at least 25 sequential voltage pulses of a substantially same voltage magnitude, after which the voltage magnitude is changed for subsequent voltage pulses.

35. The system of claim 32, wherein the combination of the at least one set of electrodes for arrangement within a body lumen while disposed within an inflatable balloon and the electric pulse generation system are configured to provide voltage pulses cause a mean pressure within the balloon over the course of IVL that approximates a substantially flat pressure profile.

36. The system of claim 32, wherein the combination of the at least one set of electrodes for arrangement within a body lumen while disposed within an inflatable balloon and the electric pulse generation system are configured to provide voltage pulses cause a mean pressure within the balloon over the course of IVL that approximates an increasing pressure profile.

37. The system of claim 32, wherein the combination of the at least one set of electrodes for arrangement within a body lumen while disposed within an inflatable balloon and the electric pulse generation system are configured to provide voltage pulses cause a mean pressure within the balloon over the course of IVL that approximates a declining-flat pressure profile.

38. The system of claim 32, wherein the combination of the at least one set of electrodes for arrangement within a body lumen while disposed within an inflatable balloon and the electric pulse generation system are configured to provide voltage pulses cause a mean pressure within the balloon over the course of IVL that approximates a sawtooth declining pressure profile.

39. The system of claim 32, wherein the combination of the at least one set of electrodes for arrangement within a body lumen while disposed within an inflatable balloon and the electric pulse generation system are configured to provide voltage pulses cause a mean pressure within the balloon over the course of IVL that approximates a sawtooth increasing pressure profile.

40. The system of claim 32, wherein the combination of the at least one set of electrodes for arrangement within a body lumen while disposed within an inflatable balloon and the electric pulse generation system are configured to provide voltage pulses cause a mean pressure within the balloon over the course of IVL that approximates a decreasing-flat-decreasing-flat pressure profile.

41. The system of claim 32, wherein the balloon is constructed from a polymeric material with a double-wall thickness of 0.0023″ or less.

42. The system of claim 32 wherein, when inflated, the balloon has a radius from an axis of the balloon of approximately 2.5 mm and a length of 20 mm or less.

43. The system of claim 32 wherein, when inflated, the balloon has a radius from an axis of the balloon of approximately 3.0 mm and a length of 20 mm or less.

44. The system of claim 32 wherein, when inflated, the balloon has a radius from an axis of the balloon of approximately 3.5 mm and a length of 20 mm or less.

45. The system of claim 32 wherein, when inflated, the balloon has a radius from an axis of the balloon of approximately 4.0 mm and a length of 20 mm or less.

46. The system of claim 41 wherein, the polymeric material is a Nylon material.

47. The system of claim 41 wherein, the polymeric material is Vestamid.

48. The system of claim 32 wherein the at least one set of electrodes comprises two pairs of electrodes, each pair of electrodes having a gap, and each pulse provides a spark in the gap between both of the pairs of electrodes.
